(19) United States
(12) Patent Application Publication
(10) Pub. No.: US 2008/0081336 A1
(43) Pub. Date: Apr. 3, 2008

(54) Variants of the Serotonin Transporter

(76) Inventors: David A. Mrazek, Rochester, MN (US); Richard M. Weinshilboum, Rochester, MN (US); Eric D. Wieben, Rochester, MN (US); Linda L. Pelleymounter, Rochester, MN (US); Irene Moon, Rochester, MN (US)

Correspondence Address: FISH & RICHARDSON P.C., PO BOX 1022, MINNEAPOLIS, MN, 55440-1022, US

(21) Appl. No.: 11/832,939

(22) Filed: Aug. 2, 2007

Related U.S. Application Data
(60) Provisional application No. 60/821,204, filed on Aug. 2, 2006.

(51) Int. Cl. C12Q 1/68; C07H 21/00; C07K 14/00; C12N 15/00

(52) U.S. Cl. 435/6; 435/320.1; 530/350; 536/23.1

(57) ABSTRACT

Isolated SLC6A4 nucleic acid molecules that include a nucleotide sequence variant and nucleotides flanking the sequence variant are described. Methods for determining whether a subject contains an SLC6A4 sequence variant also are provided.

### CROSS-REFERENCE TO RELATED APPLICATIONS

This application claims priority from U.S. Provisional Application Ser. No. 60/821,204, filed Aug. 2, 2006.

### STATEMENT AS TO FEDERALLY SPONSORED RESEARCH

This invention was made with government support under grant no. GM61388 awarded by the National Institutes of Health. The government has certain rights in the invention.

### TECHNICAL FIELD

This document relates to SLC6A4 sequence variants, as well as to methods for identifying and using such variants.

### BACKGROUND

The SLC6A4 gene (also referred to as HTT, 5HTT, OCD1, SERT, 5-HTT, and hSERT) encodes an integral membrane protein that transports the monoamine neurotransmitter serotonin (5-hydroxytryptamine) from synaptic spaces into presynaptic neurons. The encoded protein terminates the action of serotonin and recycles it in a sodium-dependent manner. The protein is a target of psychomotor stimulants, such as amphetamines and cocaine, and is a member of the sodium:neurotransmitter symporter family.

A polymorphism in the promoter region (referred to as the 5-HTT gene-linked polymorphic region, or 5-HTTLPR) is located approximately 1 kb upstream of the transcription initiation site and is composed of 16 repeat elements. The polymorphism consists of a 44-bp insertion or deletion involving repeat elements 6 to 8. The short allele is associated with lower transcriptional efficiency of the promoter as compared with the long allele. Over half of the Caucasian population has a short allele. Individuals with one or two copies of the short allele exhibit more depressive symptoms, diagnosable depression, and suicidality in relation to stressful life events than individuals homozygous for the long allele.

### SUMMARY

This document is based in part on the discovery of sequence variants in coding and non-coding regions of SLC6A4 nucleic acids. Certain SLC6A4 nucleotide sequence variants may encode proteins that are associated with individual differences in serotonin transporter activity. Other SLC6A4 sequence variants in non-coding regions of the SLC6A4 nucleic acid may alter regulation of transcription and/or splicing of the SLC6A4 nucleic acid. Discovery of these sequence variants allows individual differences in serotonin transporter activity in humans to be assessed based on the presence or absence of one or more such variants. The presence of these sequence variants also may affect responsiveness to particular therapeutic agents, and thus may be useful to indicate the potential usefulness of such agents.

In one aspect, this document features an isolated SLC6A4 nucleic acid molecule, wherein the nucleic acid molecule comprises a variant selected from the group set forth in Table 1 herein. The isolated SLC6A4 nucleic acid of molecule can be from 10 to 100 nucleotides in length (e.g., from 20 to 50 nucleotides in length). This document also features a vector comprising an isolated SLC6A4 nucleic acid molecule as set forth herein. The isolated SLC6A4 nucleic acid molecule contained within the vector can be from 20 to 50 nucleotides in length.

In another aspect, this document also features a method for determining an SLC6A4 genotype. The method can include a) providing a nucleic acid sample from a subject, and b) screening the nucleic acid sample for one or more genetic markers in linkage disequilibrium with an SLC6A4 allele. The SLC6A4 allele can be selected from the group consisting of the alleles set forth in Table 1 herein.

In another aspect, this document features a method for determining an SLC6A4 genotype, comprising a) providing a nucleic acid sample from a subject, and b) determining in the nucleic acid sample whether or not one or more genetic markers are in linkage disequilibrium with an SLC6A4 allele, wherein the SLC6A4 allele is selected from the group consisting of the alleles set forth in Table 1. The SLC6A4 allele can be selected from the group consisting of an rs25531a allele, an rs25531g allele, an rs25532c allele, and an rs25532t allele. The, SLC6A4 allele can be a 5HTTLPR short allele.

In another aspect, this document features a method for determining an SLC6A4 genotype, comprising a) providing a nucleic acid sample from a subject, and b) determining in the nucleic acid sample whether or not one or more genetic markers are in linkage disequilibrium with an SLC6A4 allele, wherein the SLC6A4 allele is selected from the group consisting of the alleles set forth in Table 3. The SLC6A4 allele can be selected from the group consisting of a G122C allele, a G167C allele, a T1393C allele, a G1462A allele, or an A1815C allele.

In still another aspect, this document features an isolated nucleic acid molecule consisting essentially of at least fifteen contiguous nucleotides of SEQ ID NO:6, wherein the sequence includes at least one nucleotide selected from the group consisting of: nucleotide 7607 of SEQ ID NO:6, with the proviso that the nucleotide at position 7607 is thymine; nucleotide 7618 of SEQ ID NO:6, with the proviso that the nucleotide at position 7618 is cytosine; nucleotide 7653 of SEQ ID NO:6, with the proviso that the nucleotide at position 7653 is adenine; nucleotide 7714 of SEQ ID NO:6, with the proviso that the nucleotide at position 7714 is thymine; nucleotide 7762 of SEQ ID NO:6, with the proviso that the nucleotide at position 7762 is cytosine; nucleotide 10308 of SEQ ID NO:6, with the proviso that the nucleotide at position 10308 is thymine; nucleotide 10748 of SEQ ID NO:6, with the proviso that the nucleotide at position 10748 is adenine; nucleotide 10929 of SEQ ID NO:6, with the proviso that the nucleotide at position 10929 is thymine; nucleotide 24133 of SEQ ID NO:6, with the proviso that the nucleotide at position 24133 is adenine; nucleotide 24213 of SEQ ID NO:6, with the proviso that the nucleotide at position 24213 is cytosine; nucleotide 24953 of SEQ ID NO:6, with the proviso that the nucleotide at position 24953 is cytosine; nucleotide 25134 of SEQ ID NO:6, with the proviso that the nucleotide at position 25134 is cytosine; nucleotide 27926 of SEQ ID NO:6, with the proviso that the nucleotide at position 27926 is thymine; nucleotide 28004 of SEQ ID NO:6, with the proviso that the nucleotide at position 28004 is thymine; nucleotide 28682 of SEQ ID NO:6, with the proviso that the nucleotide at position 28682 is thymine; nucleotide 29386 of SEQ ID NO:6, with the proviso that the nucleotide at position 29386 is adenine; nucleotide 29400 of SEQ ID NO:6, with the proviso that the nucleotide at position 29400 is thymine; nucleotide 29401 of SEQ ID NO:6, with the proviso that the nucleotide at position 29401 is adenine; nucleotide 30775 of SEQ ID NO:6, with the proviso that the nucleotide at position 30775 is cytosine; nucleotide 30961 of SEQ ID NO:6, with the proviso that the nucleotide at position 30961 is thymine; nucleotide 31039 of SEQ ID NO:6, with the proviso that the nucleotide at position 31039 is thymine; nucleotide 33899 of SEQ ID NO:6, with the proviso that the nucleotide at position 33899 is adenine; nucleotide 33995 of SEQ ID NO:6, with the proviso that the nucleotide at position 33995 is thymine; nucleotide 34254 of SEQ ID NO:6, with the proviso that the nucleotide at position 34254 is guanine; nucleotide 34270 of SEQ ID NO:6, with the proviso that the nucleotide at position 34270 is thymine; nucleotide 36119 of SEQ ID NO:6, with the proviso that the nucleotide at position 36119 is adenine; nucleotide 36344 of SEQ ID NO:6, with the proviso that the nucleotide at position 36344 is thymine; nucleotide 37560 of SEQ ID NO:6, with the proviso that the nucleotide at position 37560 is adenine; nucleotide 38827 of SEQ ID NO:6, with the proviso that the nucleotide at position 38827 is thymine; and nucleotide 38840 of SEQ ID NO:6, with the proviso that the nucleotide at position 38840 is thymine; or the complement of the sequence. The isolated nucleic acid molecule can be 15 to 100 nucleotides in length, or 20 to 50 nucleotides in length.

This document also features a vector comprising an isolated nucleic acid molecule as described above. The nucleic acid molecule can be 20 to 50 nucleotides in length.

In another aspect, this document features an isolated nucleic acid encoding a SLC6A4 polypeptide comprising an amino acid sequence variant relative to the amino acid sequence set forth in SEQ ID NO:3, wherein the amino acid sequence variant is at residue 122 or residue 1462. The amino acid sequence variant can be an alanine at residue 122 or a methionine at residue 1462.

In yet another aspect, this document features an isolated SLC6A4 polypeptide, wherein the polypeptide comprises an amino acid sequence variant relative to the amino acid sequence set forth in SEQ ID NO:3, and wherein the amino acid sequence variant is at residue 122 or 1462. The amino acid sequence variant can be an alanine at residue 122 or a methionine at residue 1462.

In another aspect, this document features an article of manufacture comprising a substrate, wherein the substrate comprises a population of isolated nucleic acid molecules as described above. The substrate can comprise a plurality of discrete regions, wherein each region comprises a different population of isolated nucleic acid molecules, and wherein each population of molecules comprises a different SLC6A4 nucleotide sequence variant.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a nucleotide sequence of a reference SLC6A4 nucleic acid (SEQ ID NO:1).

FIG. 2 is a nucleotide sequence of a reference SLC64A cDNA sequence (SEQ ID NO:2).

FIG. 3 is an amino acid sequence of a reference SLC6A4 polypeptide (SEQ ID NO:3).

FIG. 4 is a diagram showing locations of polymorphisms within the SLC6A4 gene.

FIG. 5 shows a nucleotide sequence of a reference SLC6A4 genomic nucleic acid sequence (SEQ ID NO:6), and an amino acid sequence of a reference SLC6A4 polypeptide (SEQ ID NO:3).

FIG. 6 shows a nucleic acid sequence of a reference SLC6A4 cDNA sequence (SEQ ID NO:7), and an amino acid sequence of a reference SLC6A4 polypeptide (SEQ ID NO:3).

### DETAILED DESCRIPTION

This document discloses SLC6A4 nucleotide sequence variants. SLC6A4 encodes the serotonin neurotransmitter transporter, an integral membrane protein that transports serotonin from synaptic spaces into presynaptic neurons, terminating the action of serotonin and recycling it in a sodium-dependent manner. The protein is a target of psychomotor stimulants such as amphetamines and cocaine. Certain SLC6A4 nucleotide sequence variants, in coding regions or in non-coding regions, are associated with individual differences in serotonin transporter activity. Discovery of these sequence variants allows individual differences in serotonin transporter activity in humans to be assessed based on the presence or absence of one or more such variants.

Nucleic Acid Molecules

The isolated nucleic acids provided herein can include an SLC6A4 nucleic acid sequence. The SLC6A4 nucleic acid sequence can include a nucleotide sequence variant and nucleotides flanking the sequence variant. As used herein, “isolated nucleic acid” refers to a nucleic acid that is separated from other nucleic acid molecules that are present in a mammalian genome, including nucleic acids that normally flank one or both sides of the nucleic acid in a mammalian genome (e.g., nucleic acids that encode non-SLC6A4 proteins). The term “isolated” as used herein with respect to nucleic acids also includes any non-naturally-occurring nucleic acid sequence since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

Nucleic acids provided herein are at least about 8 nucleotides in length. For example, a nucleic acid can be about 8, 9, 10-20 (e.g., 11, 12, 13, 14, 15, 16, 17, 18, 19, or 20 nucleotides in length), 20-50, 50-100 or greater than 100 nucleotides in length (e.g., 150, 200, 250, 300, 350, 400, 450, 500, 750, or 1000 nucleotides in length). Nucleic acids can be in a sense or antisense orientation, can be complementary to the SLC6A4 reference sequence, and can be DNA, RNA, or nucleic acid analogs. Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine or 5-bromo-2′-doxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, Summerton and Weller, Antisense Nucleic Acid Drug Dev. (1997) 7(3): 187-195; and Hyrup et al (1996) Bioorgan. Med. Chem. 4(1):5-23. In addition, the deoxyphosphate backbone can be replaced with, for example, a phosphorothioate or phosphorodithioate backbone, a phosphoroamidite, or an alkyl phosphotriester backbone.

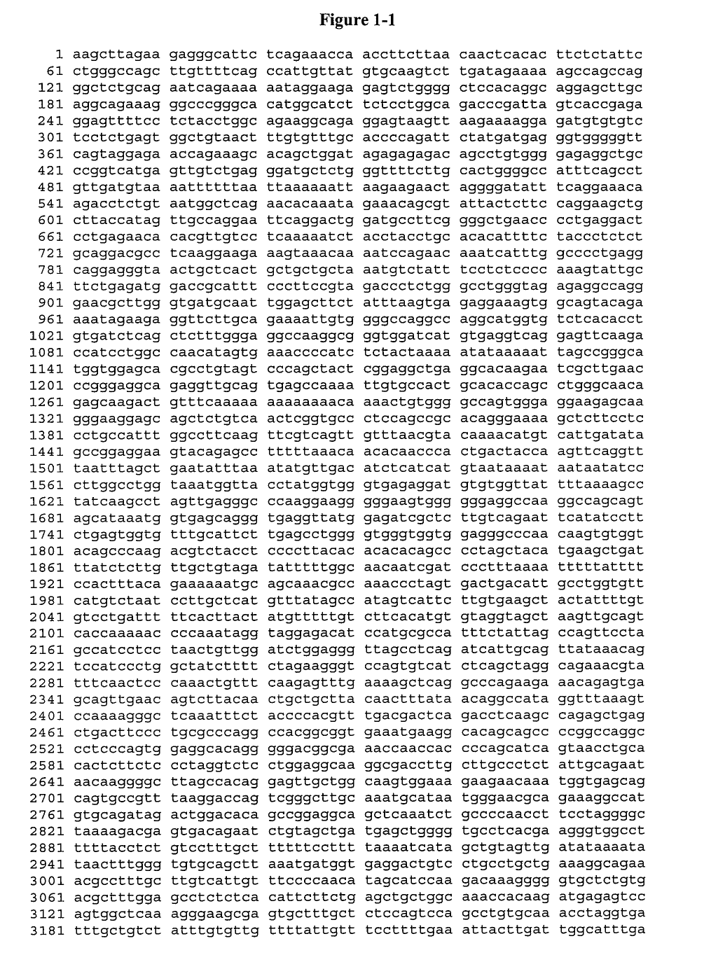

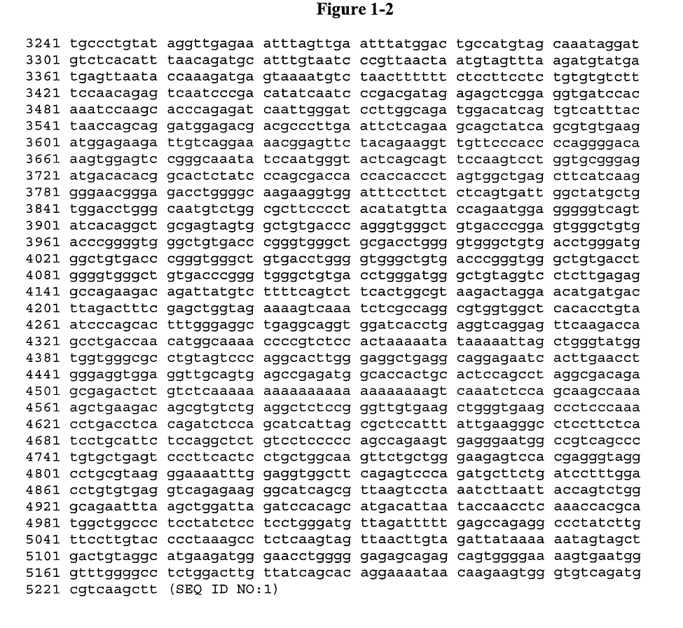

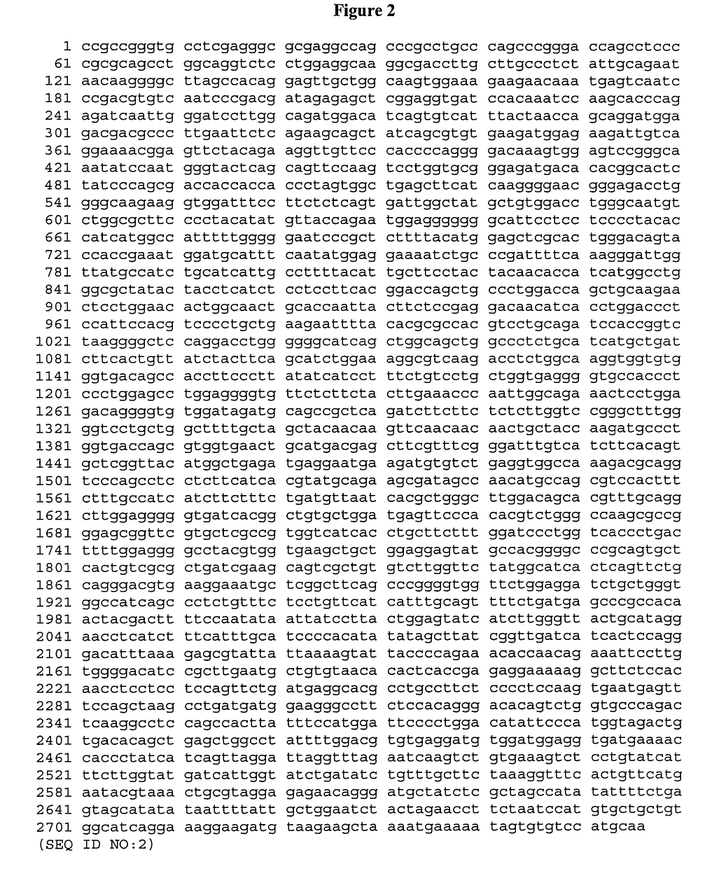

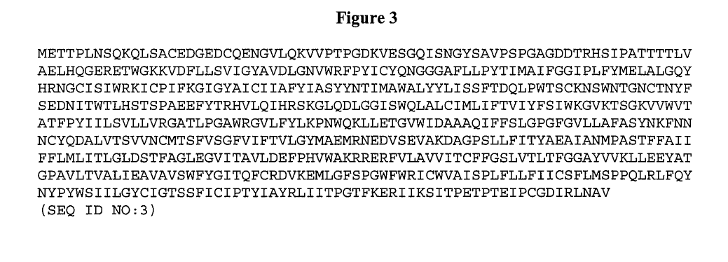

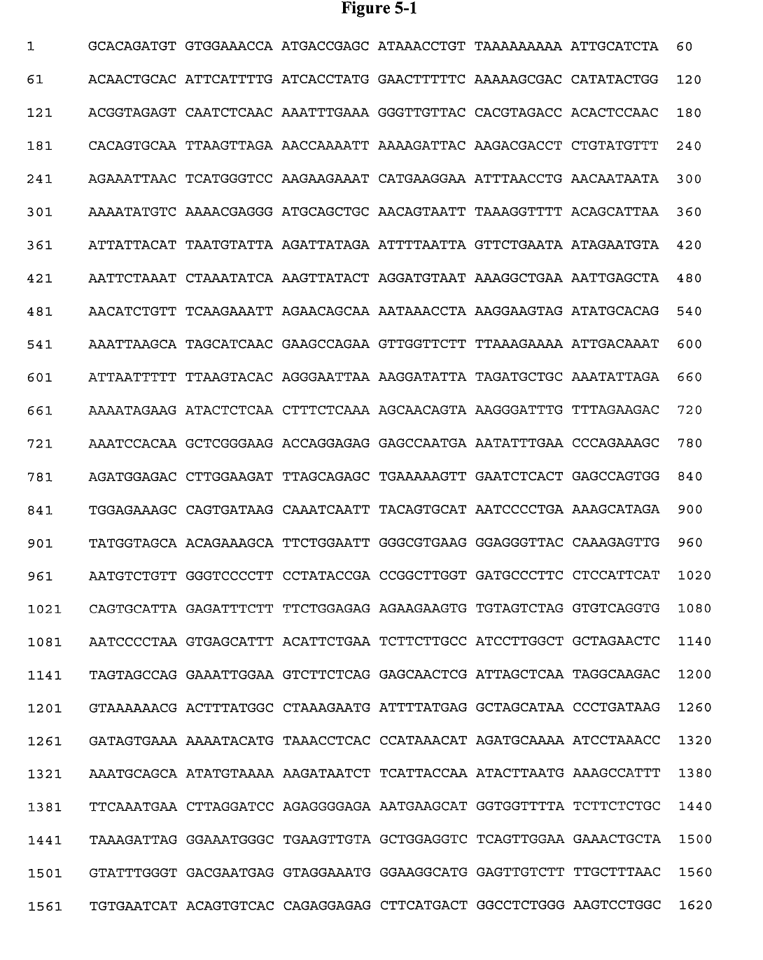

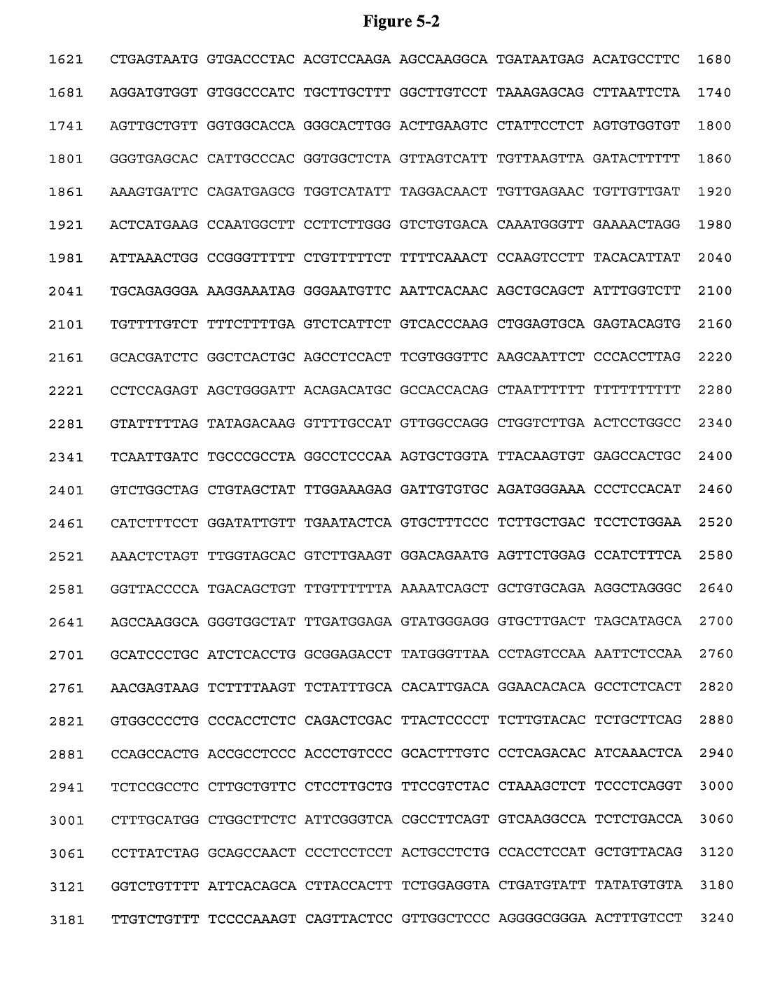

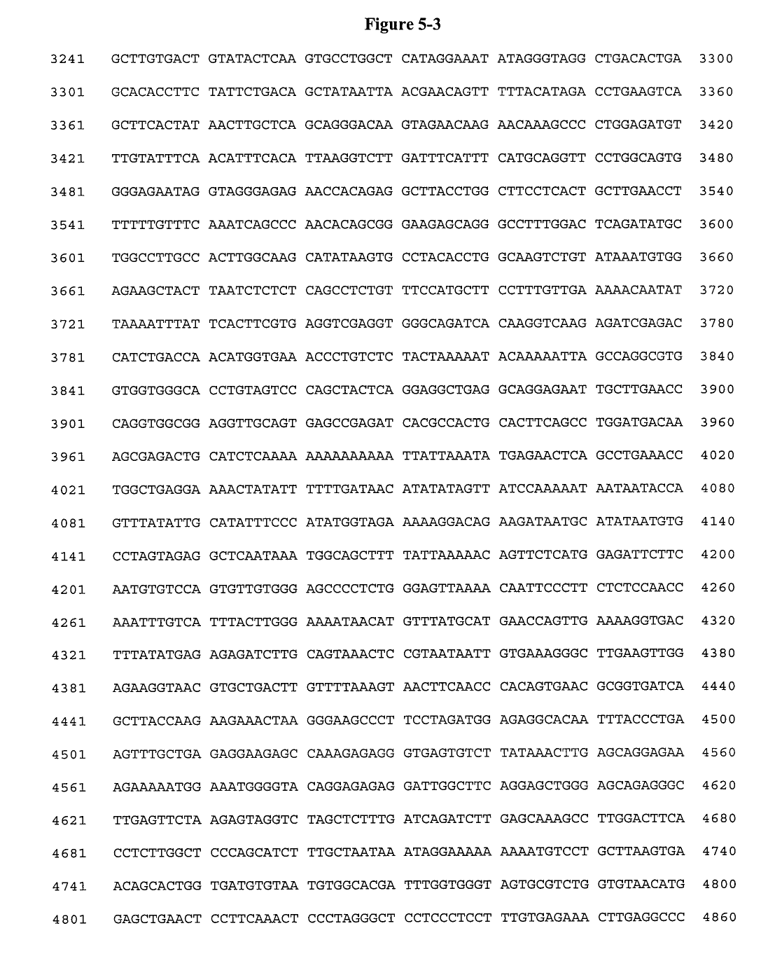

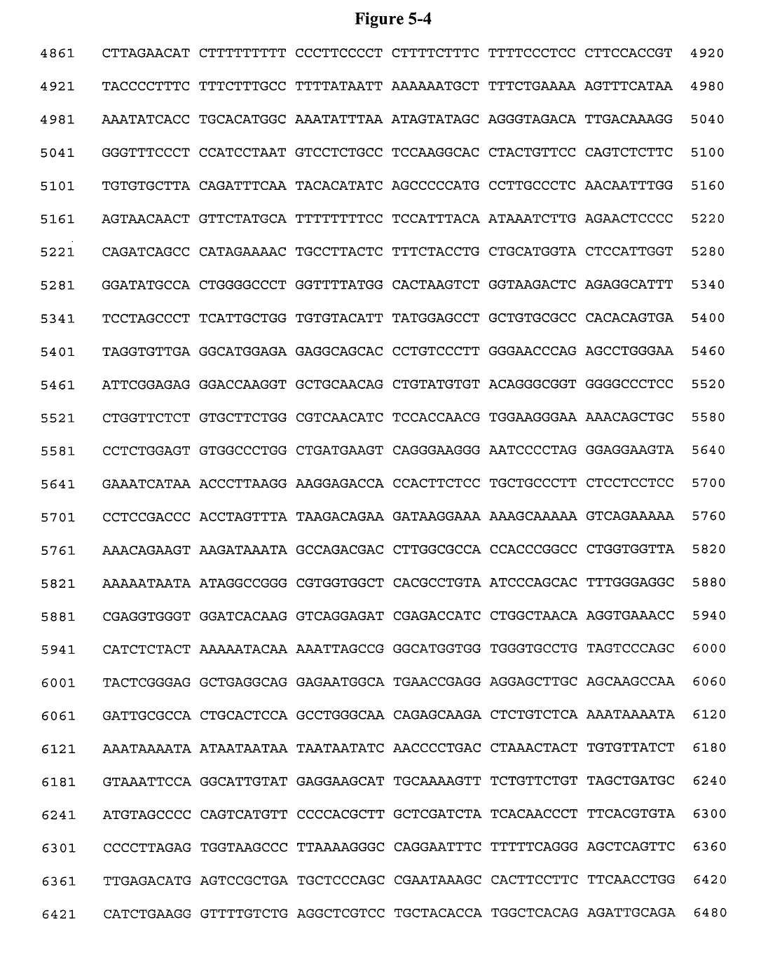

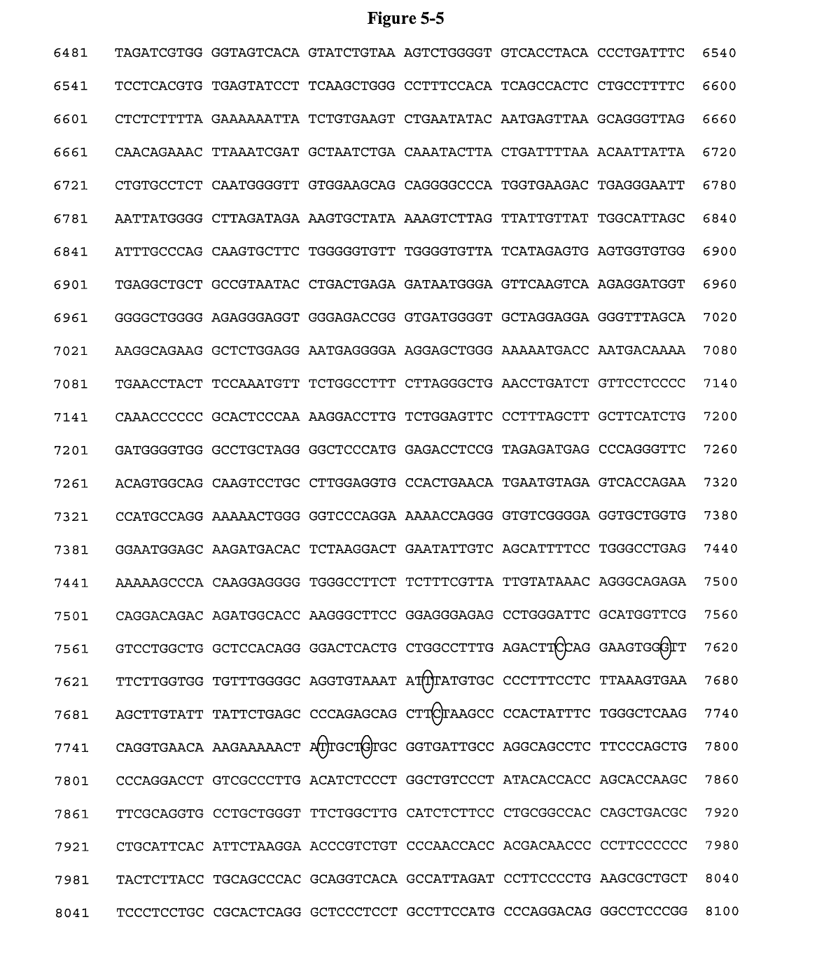

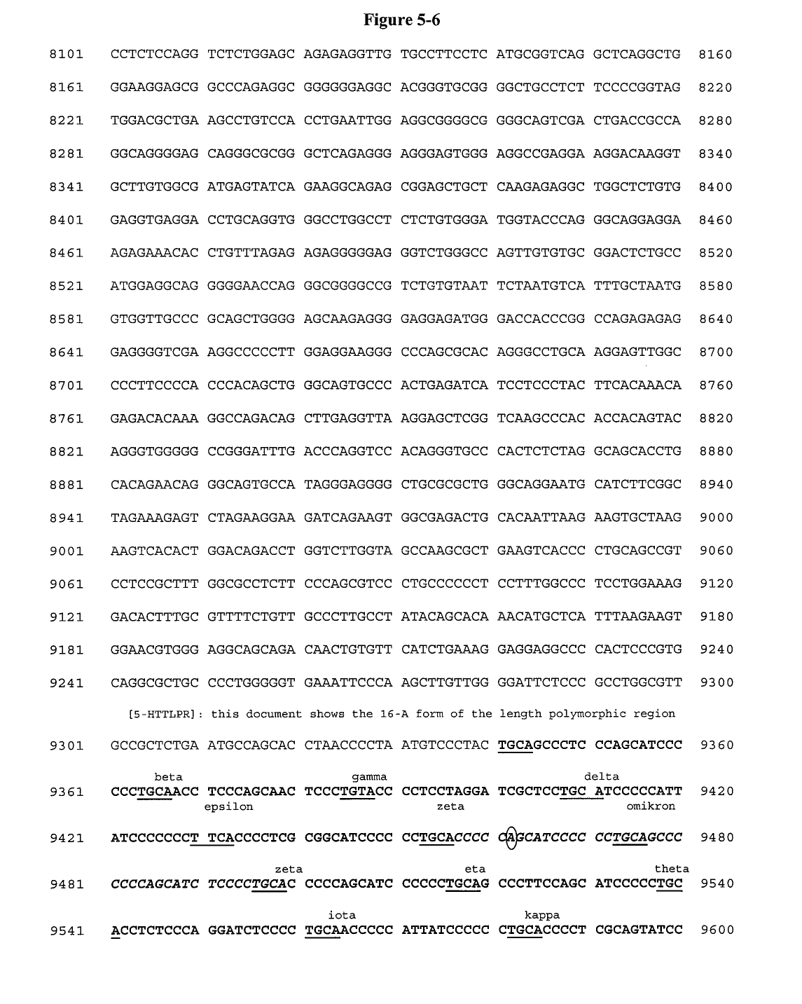

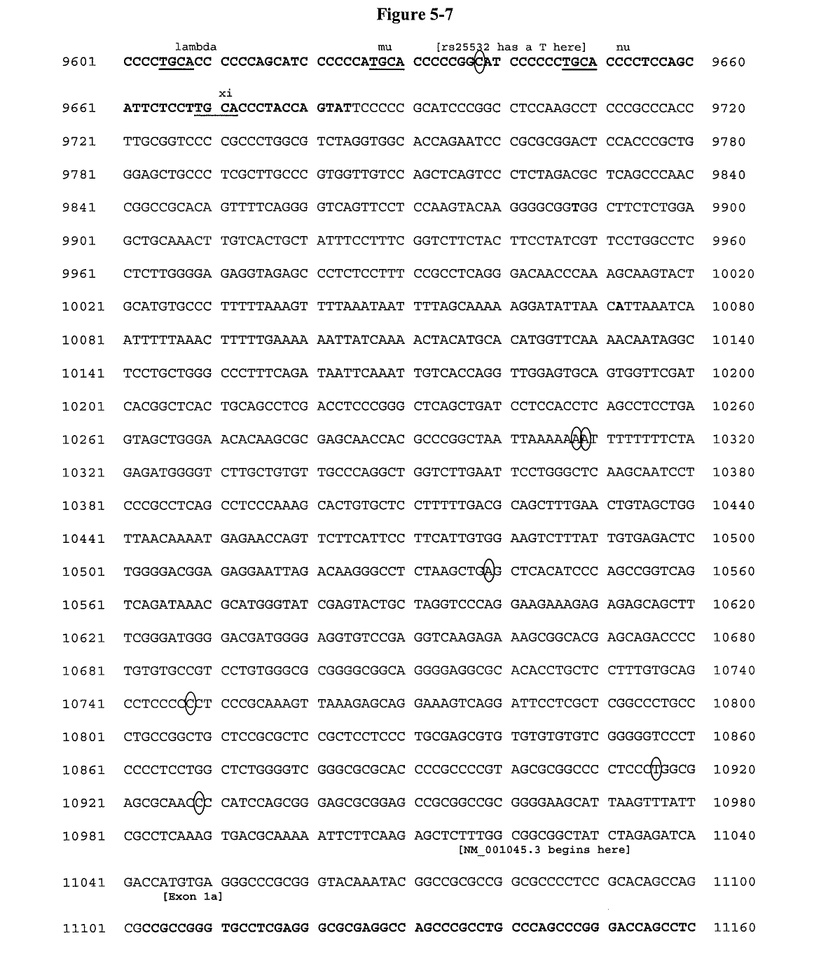

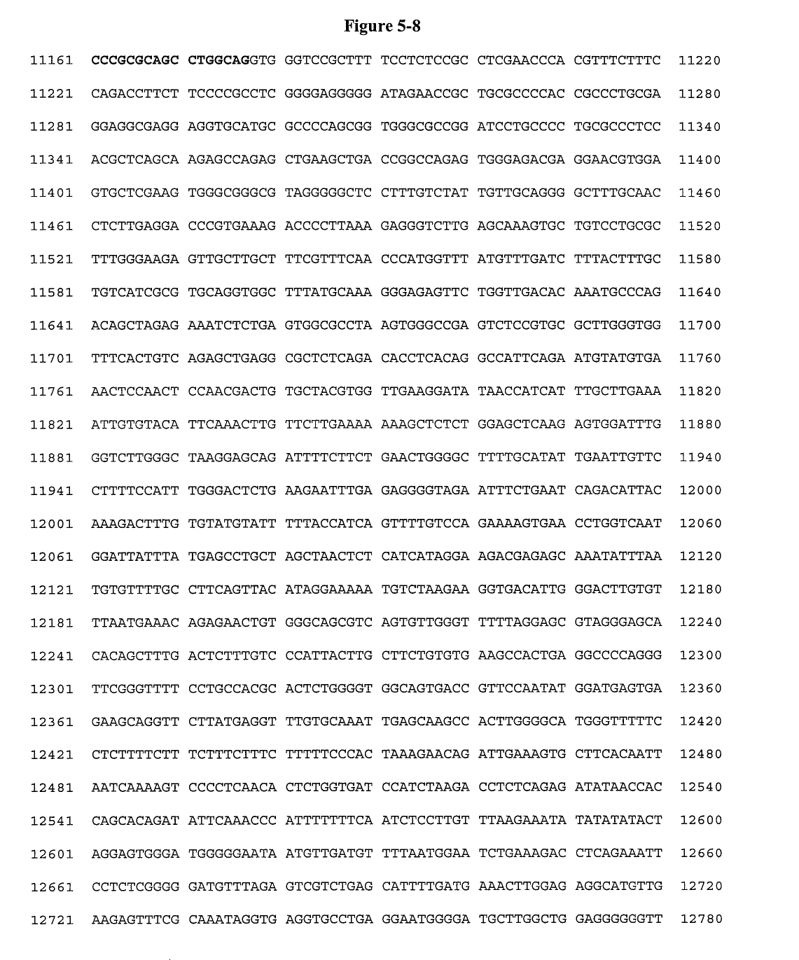

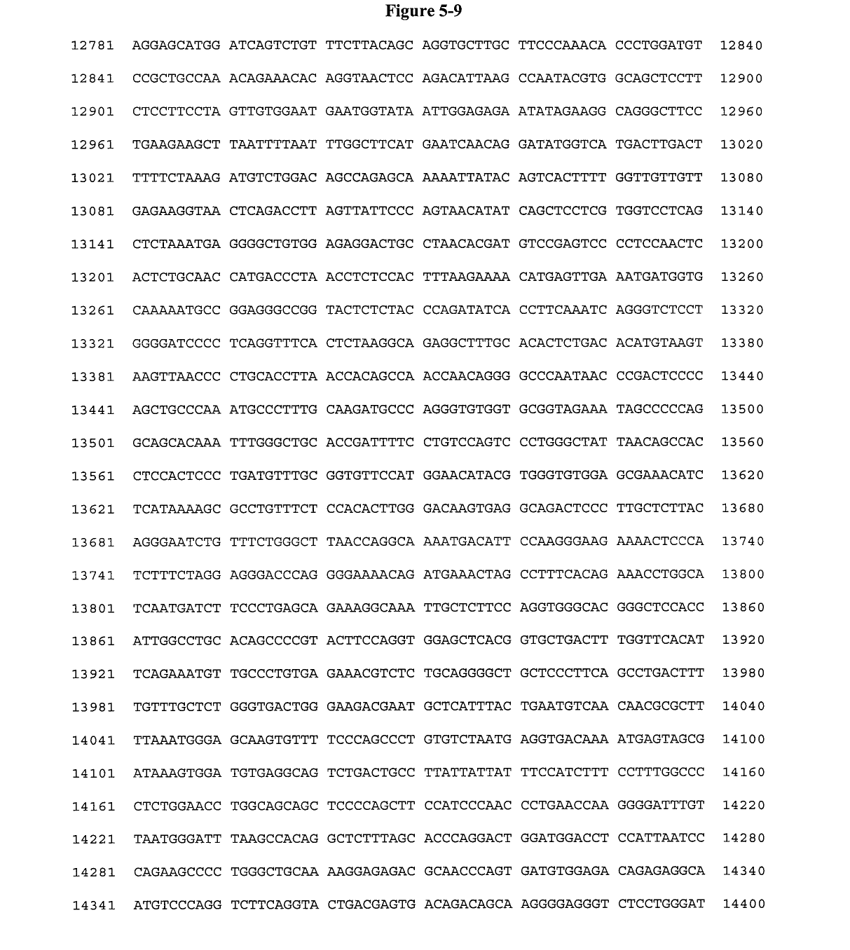

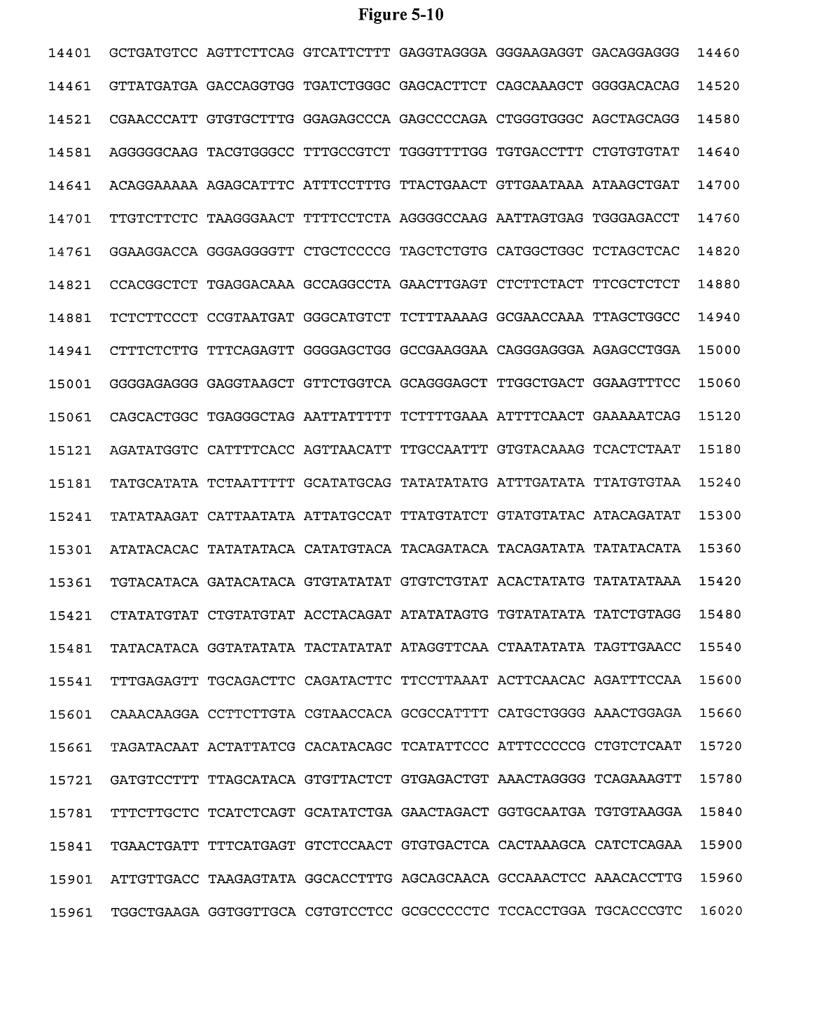

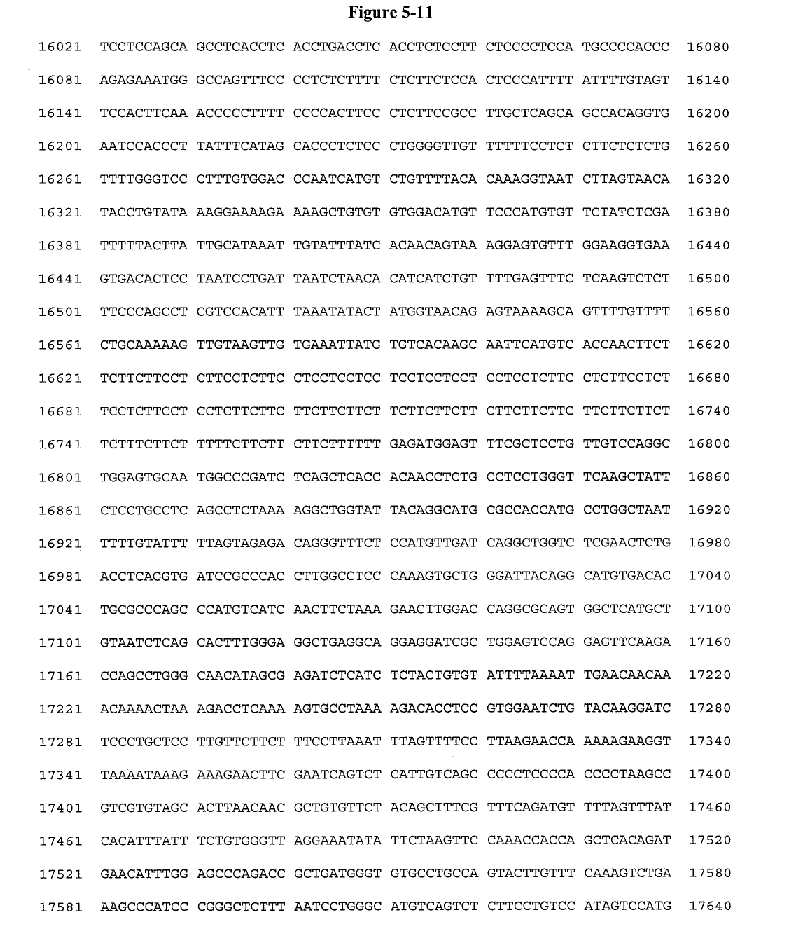

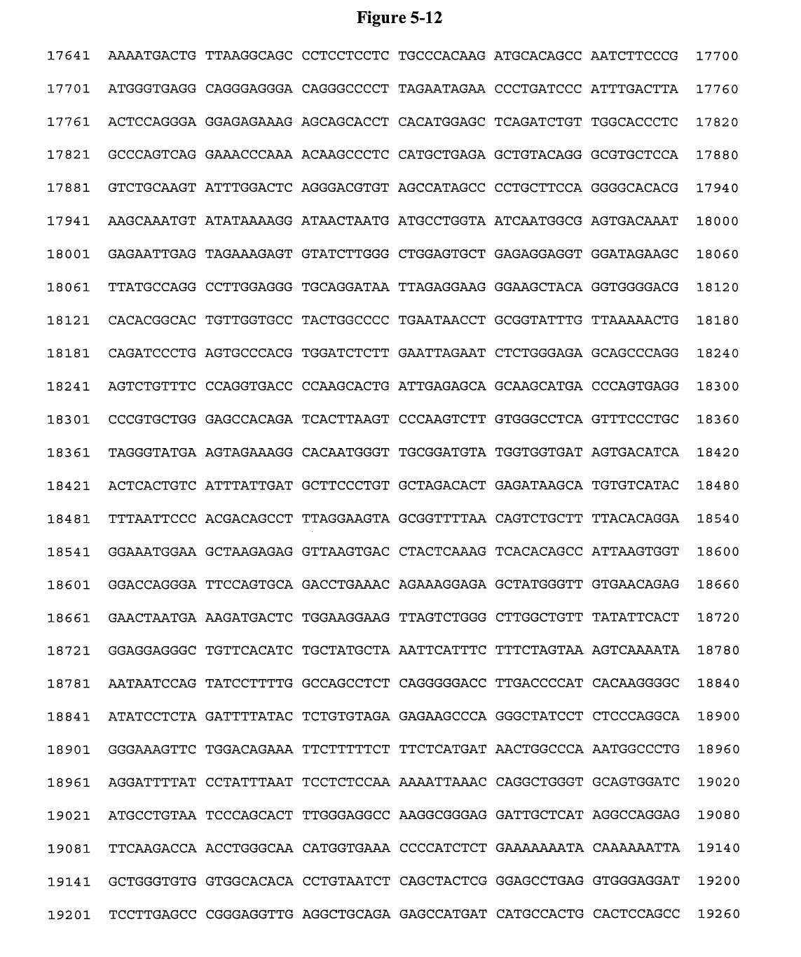

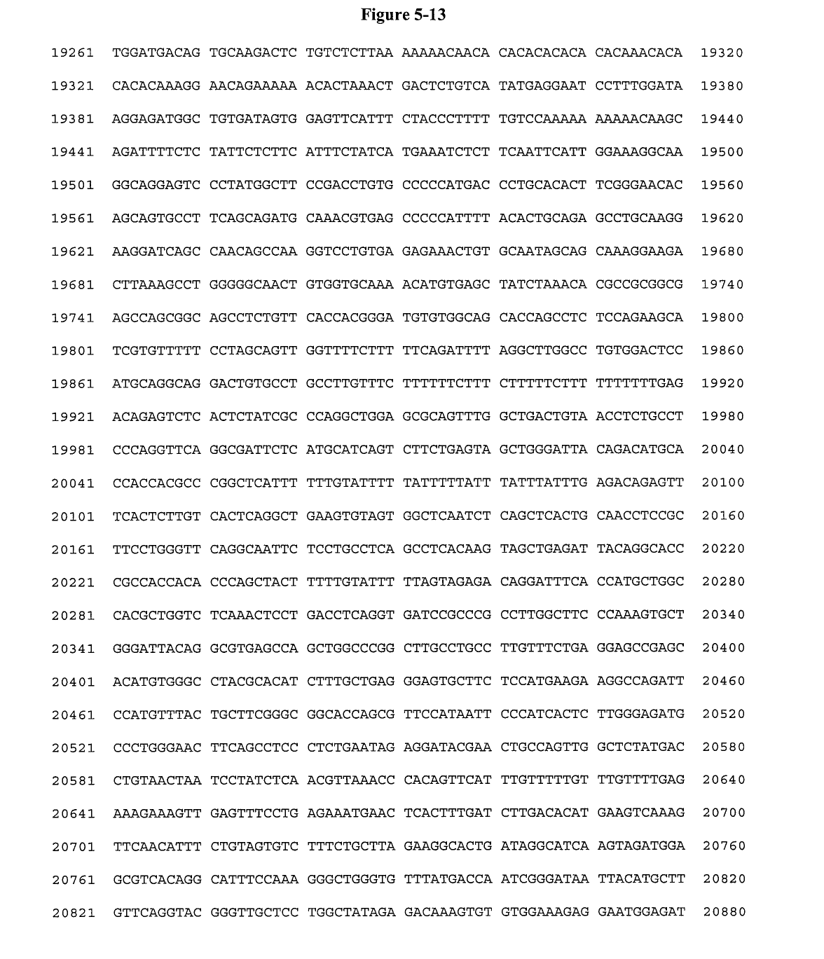

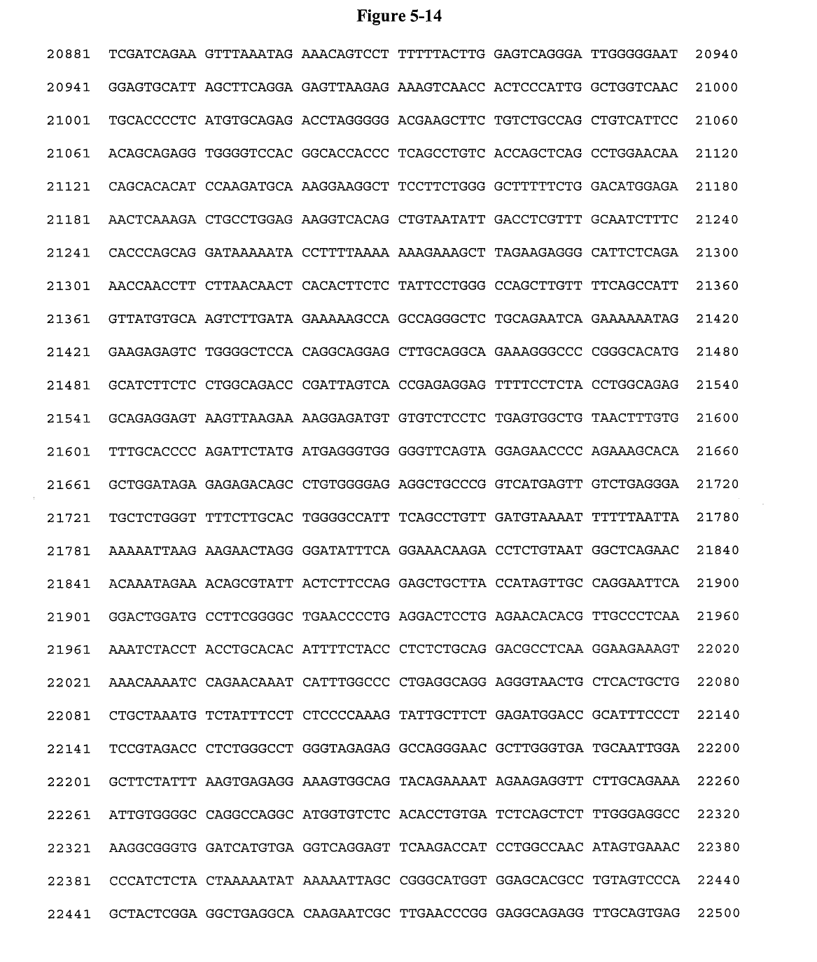

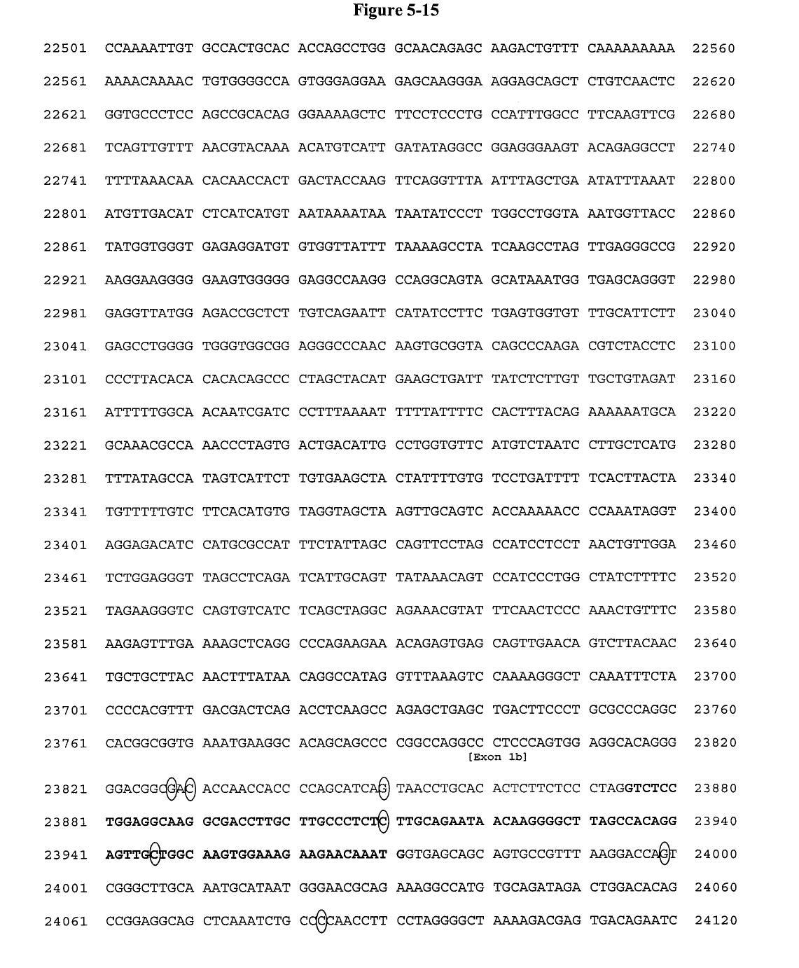

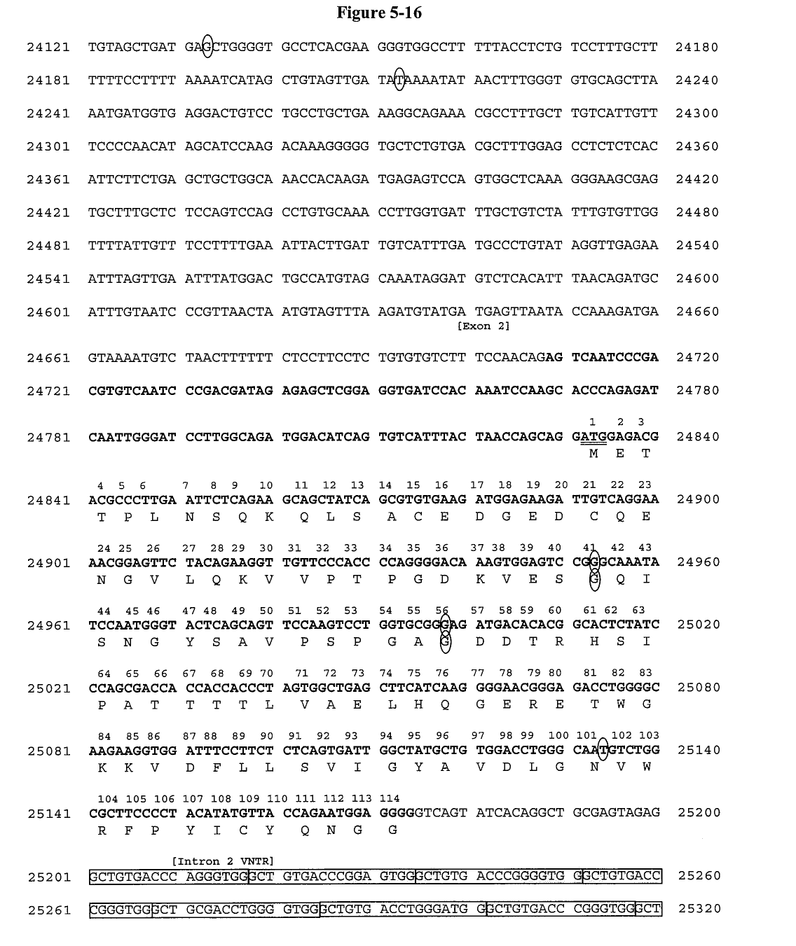

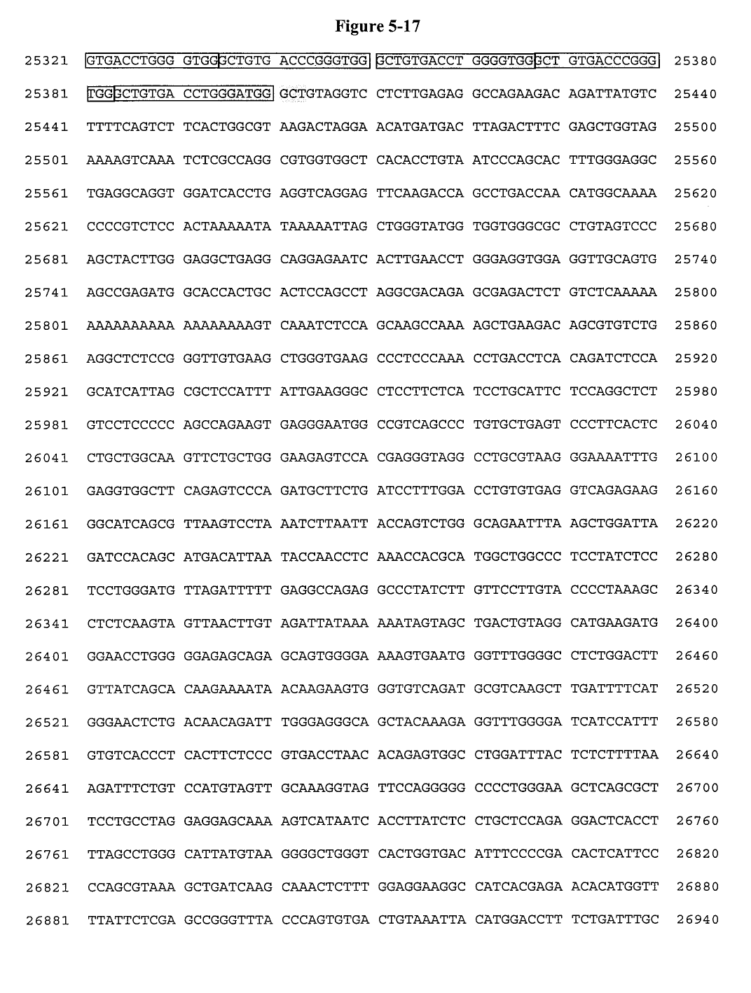

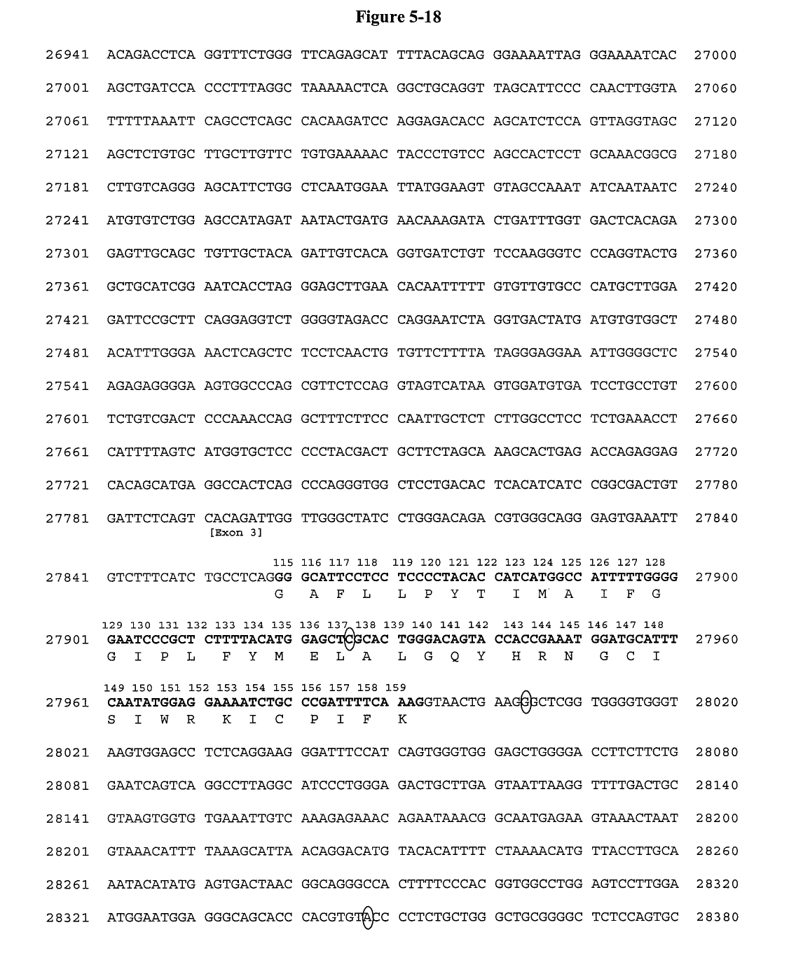

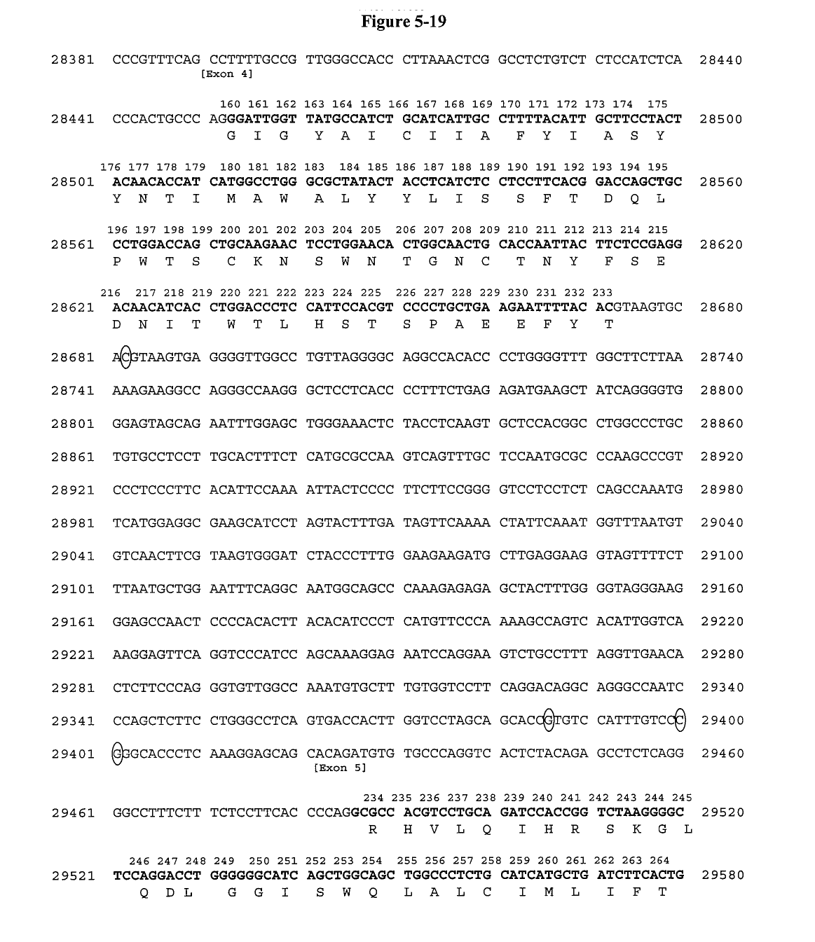

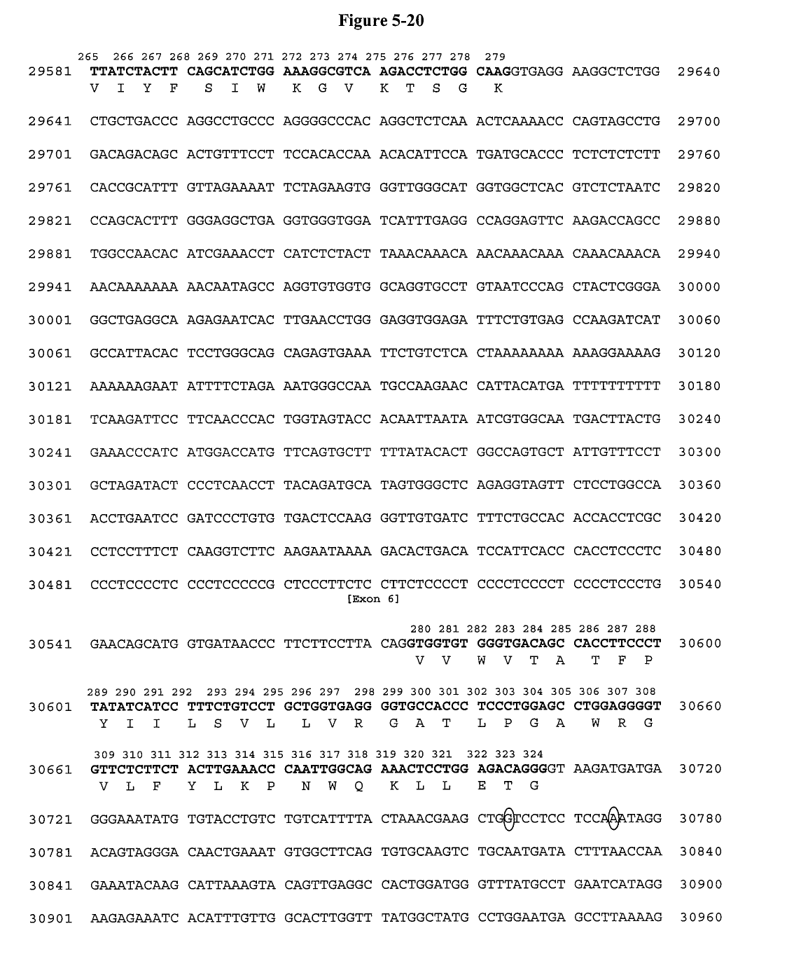

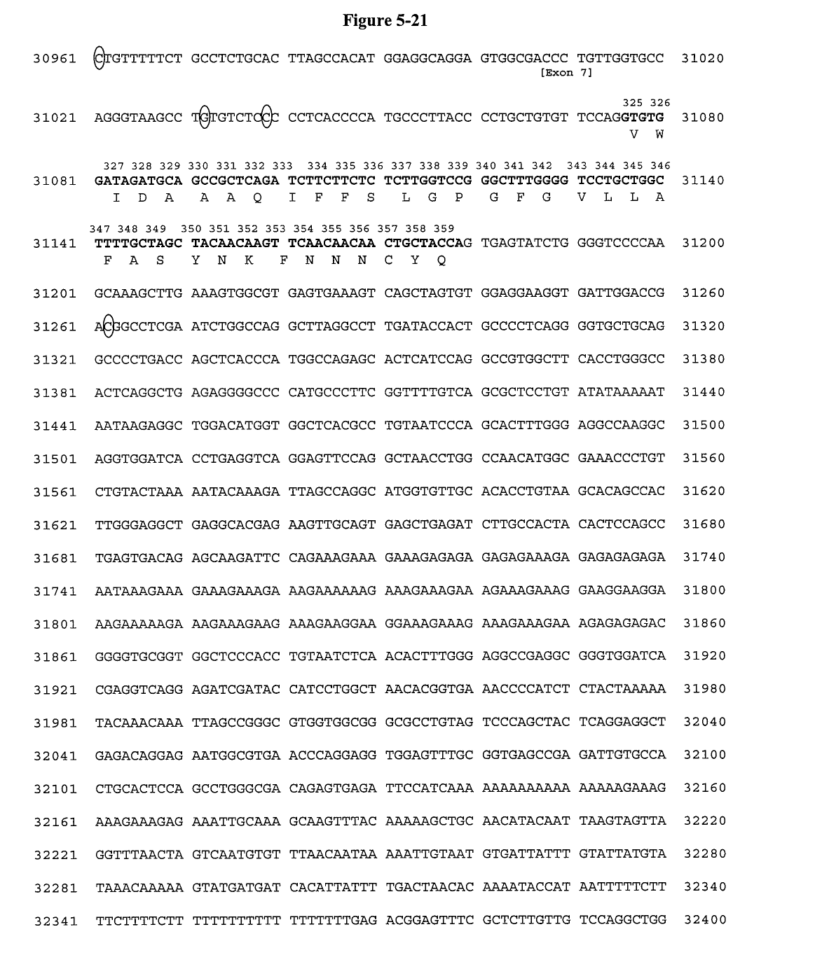

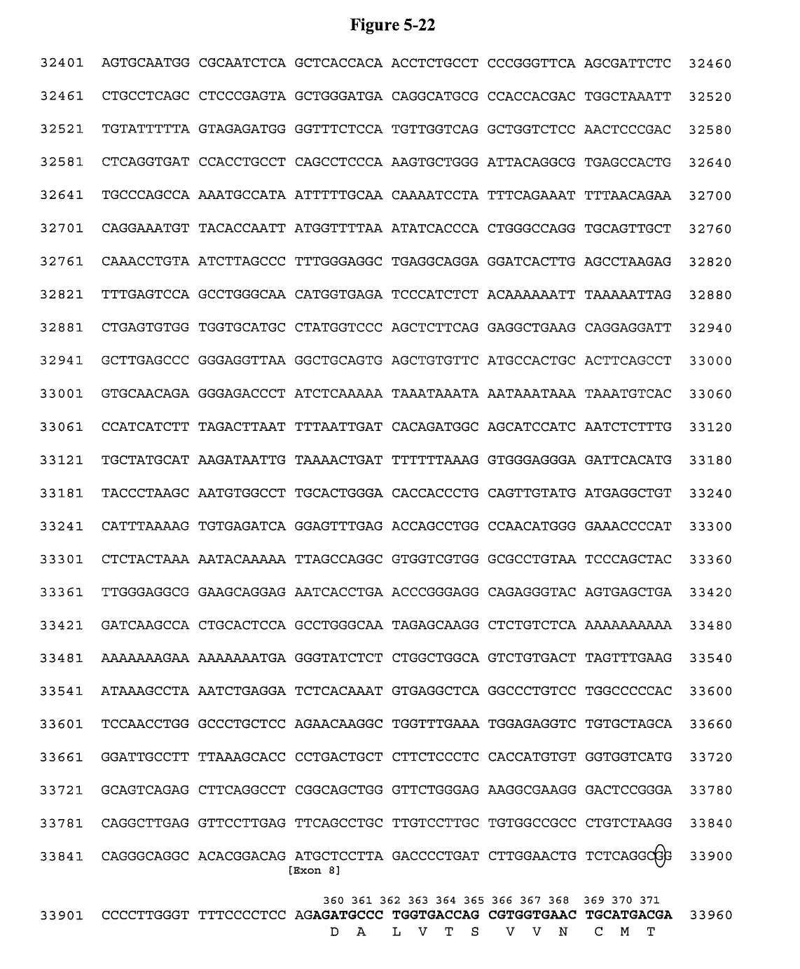

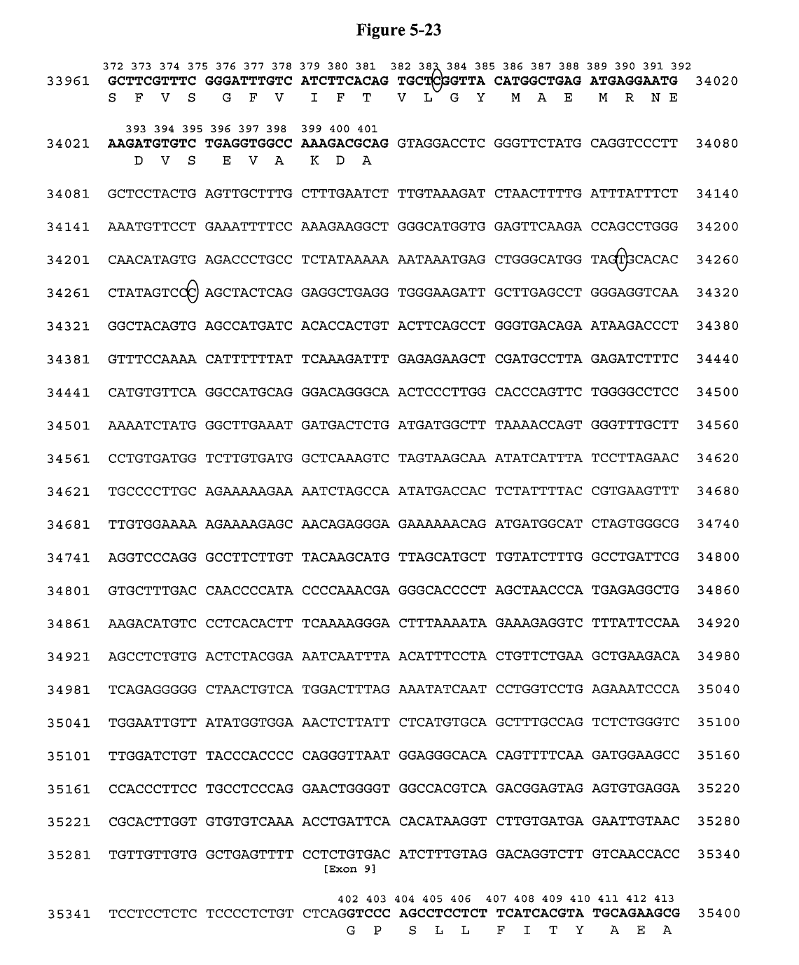

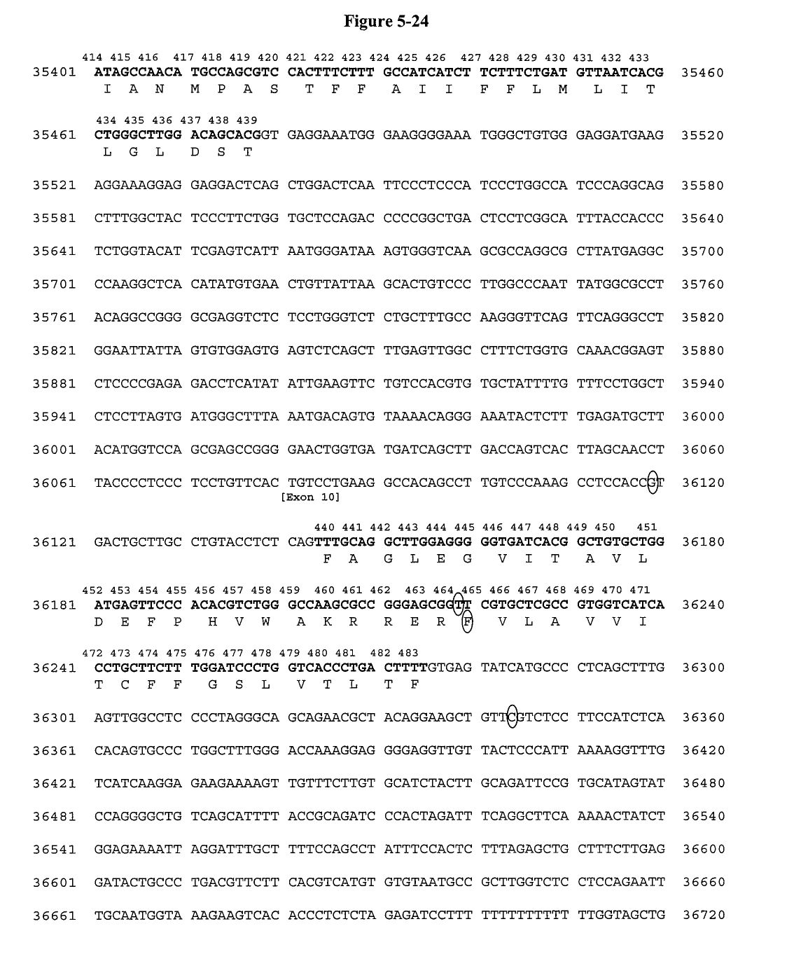

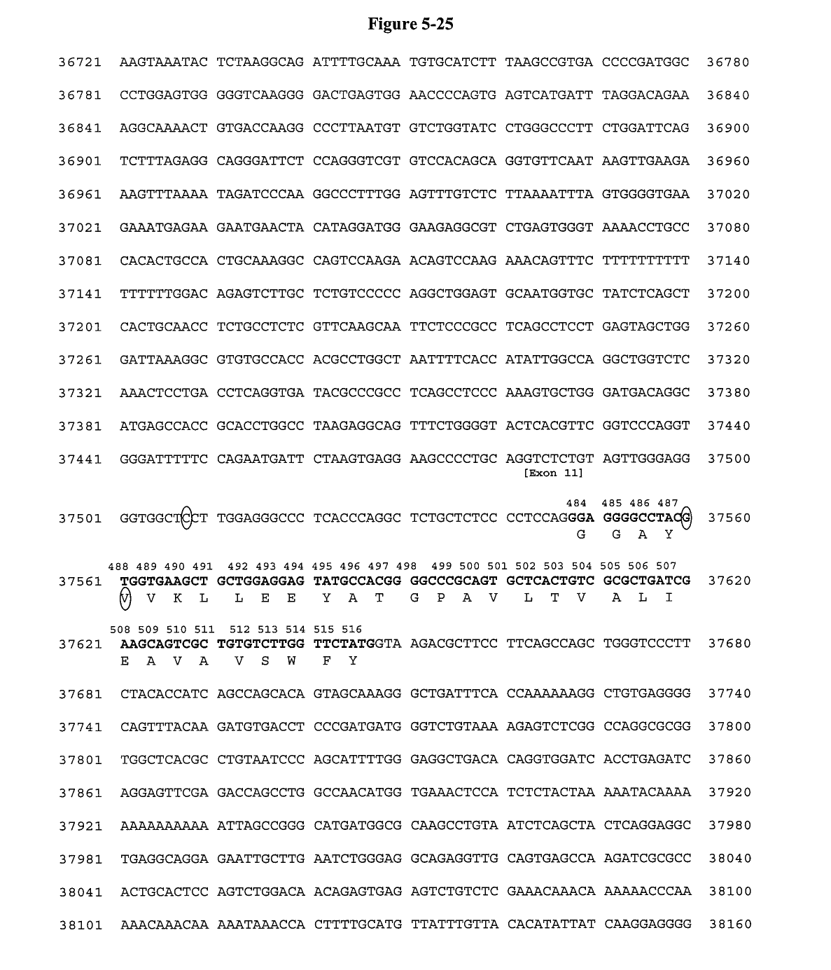

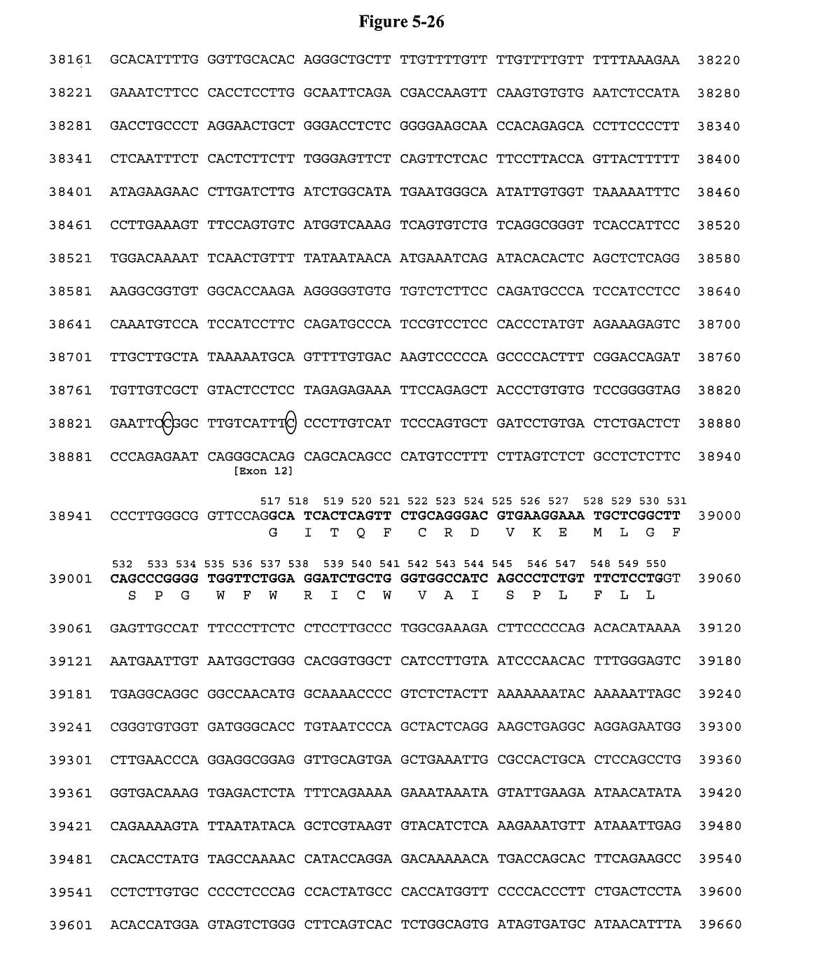

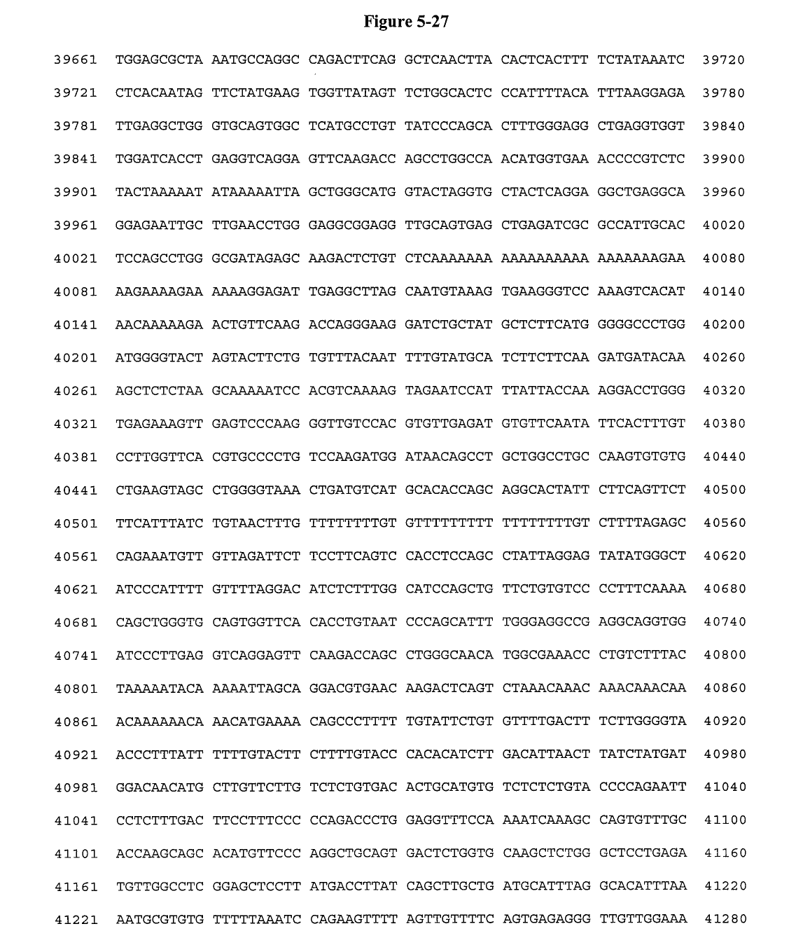

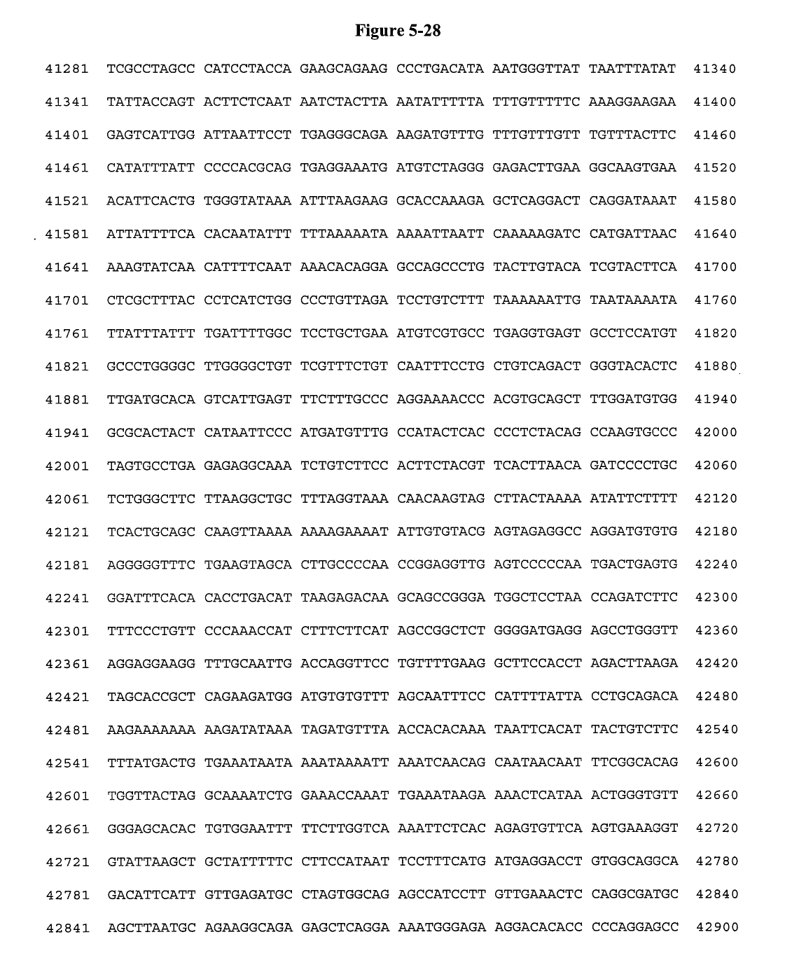

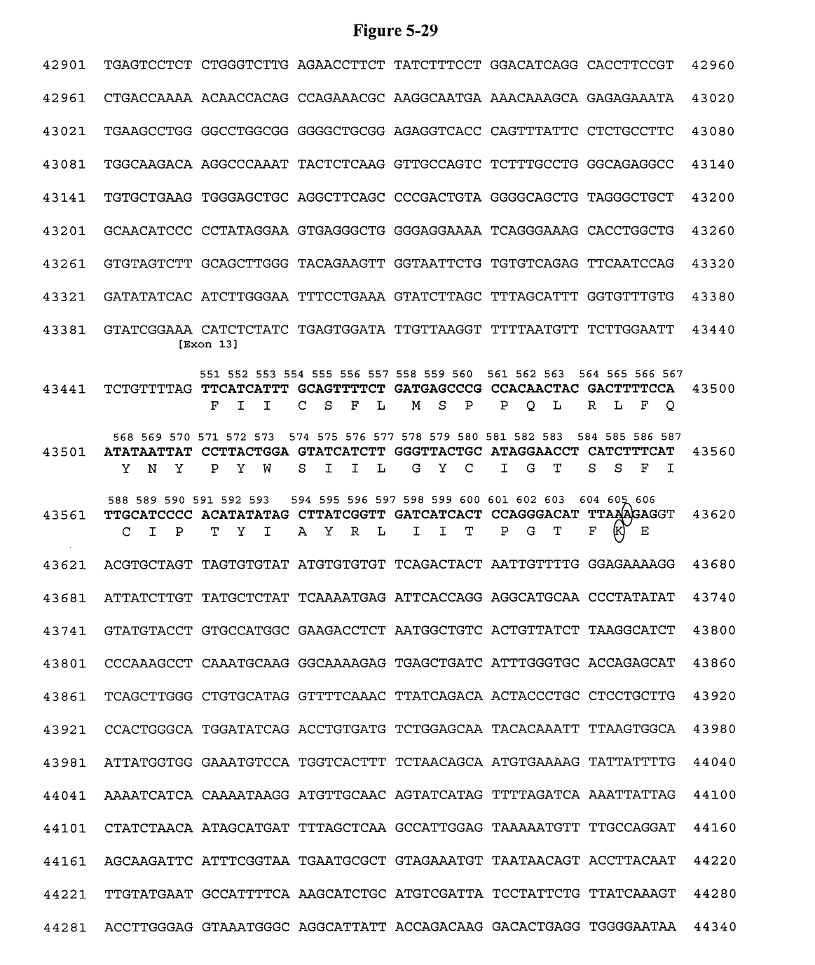

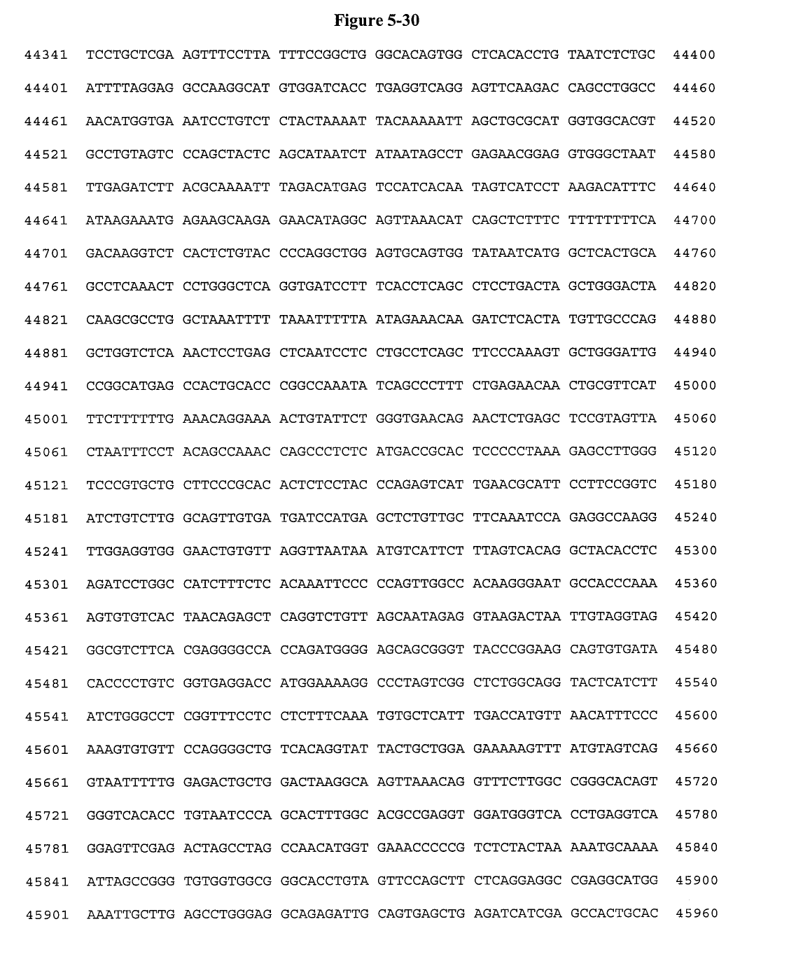

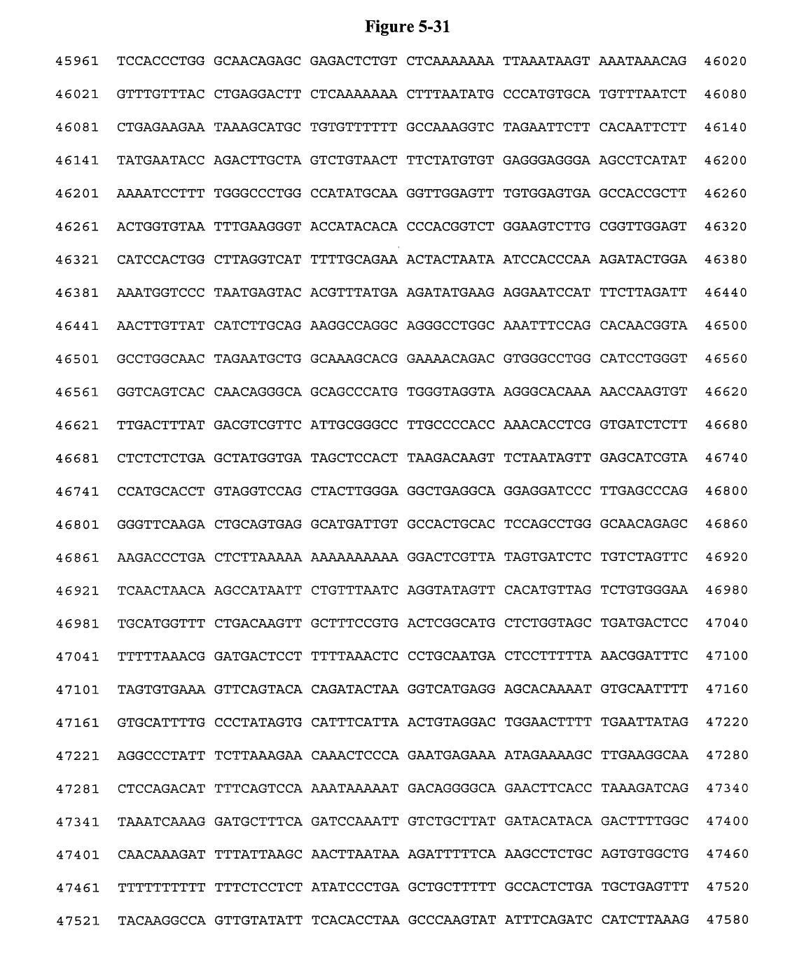

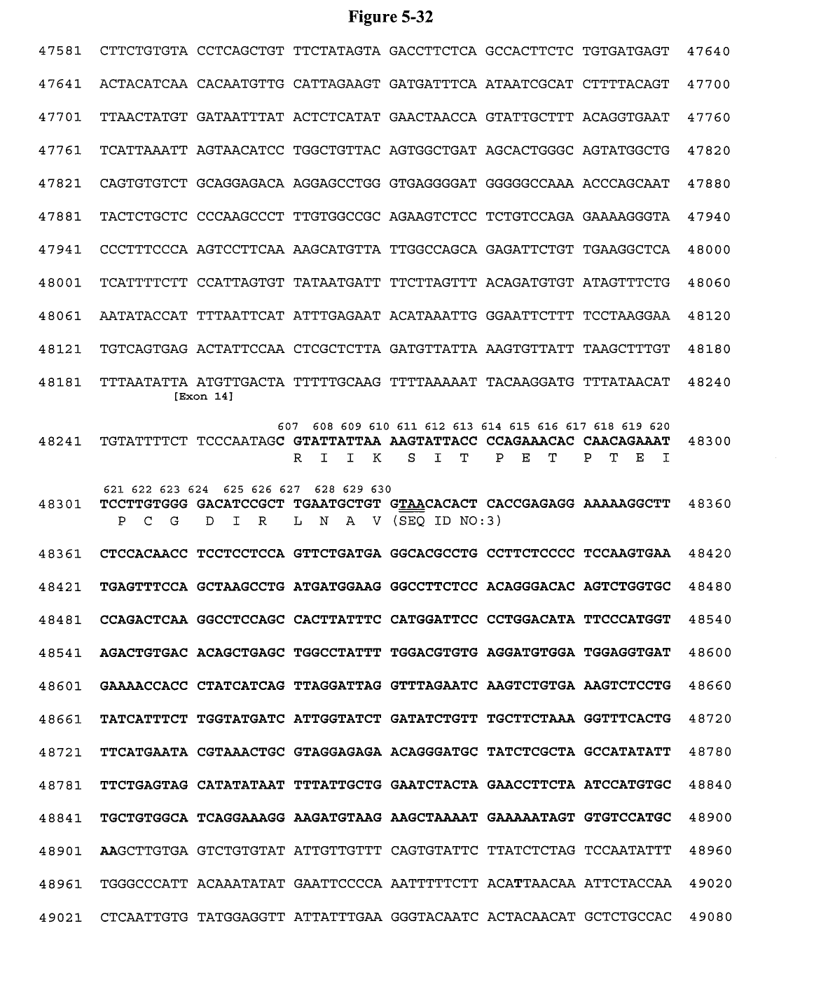

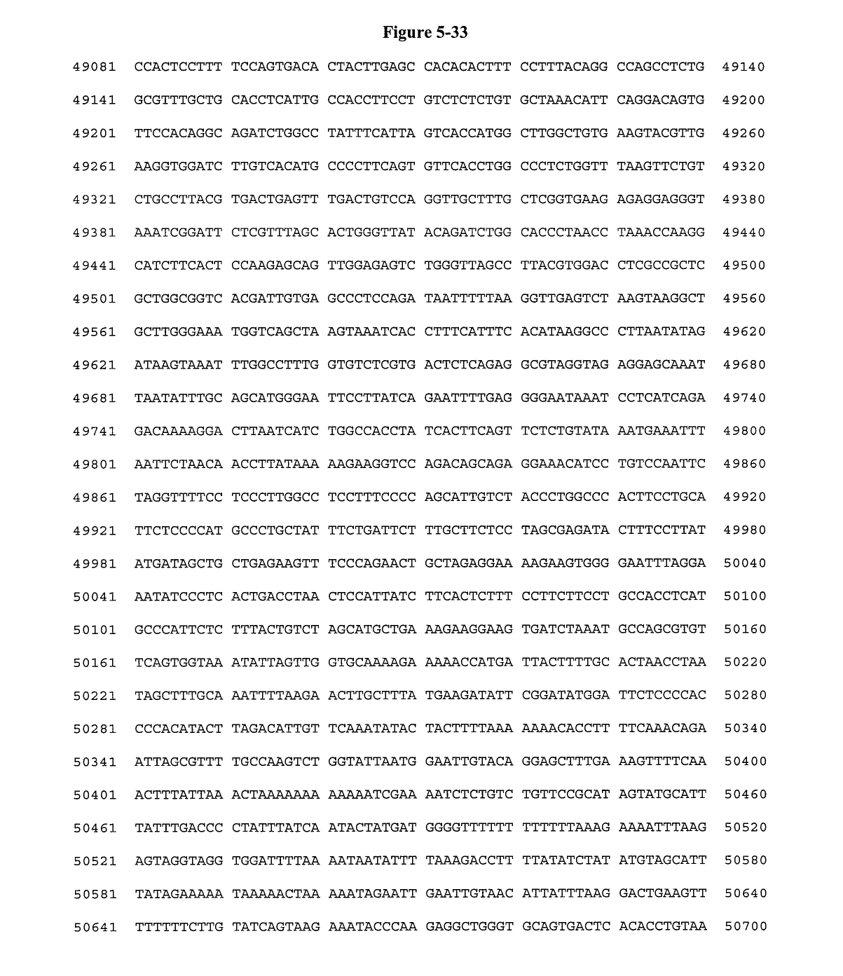

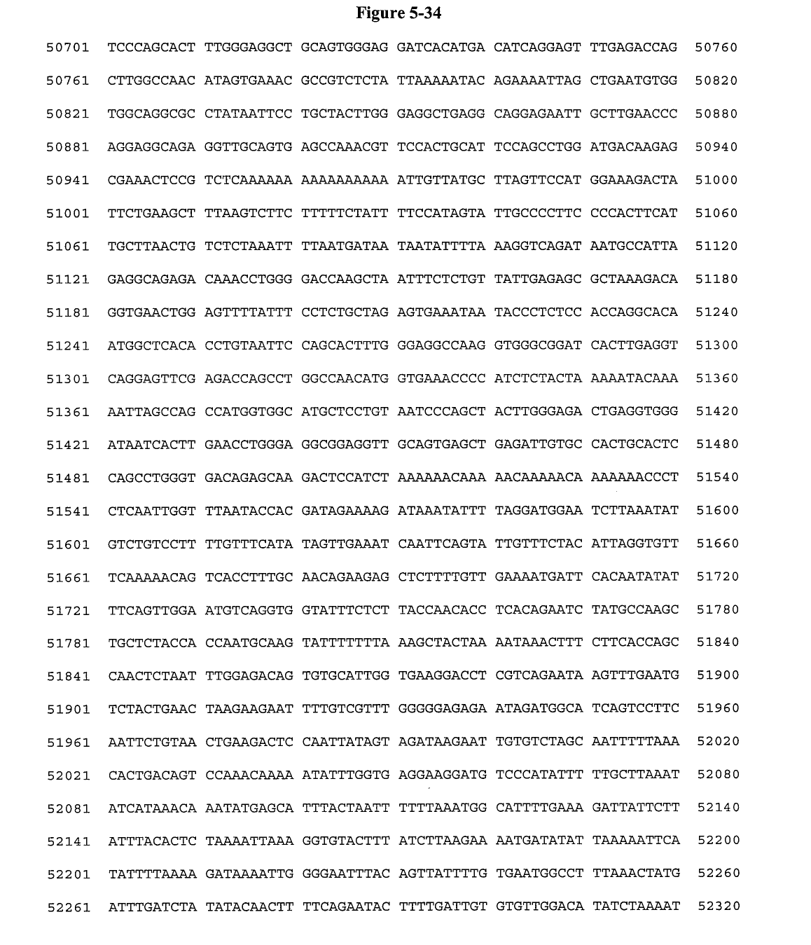

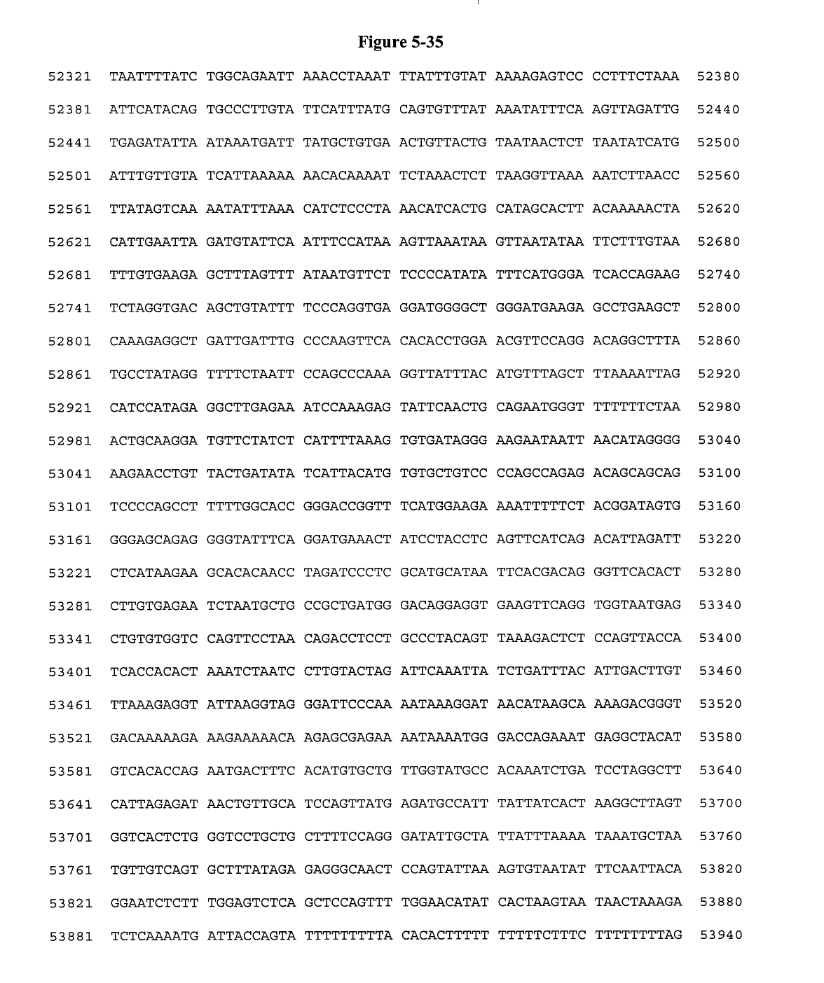

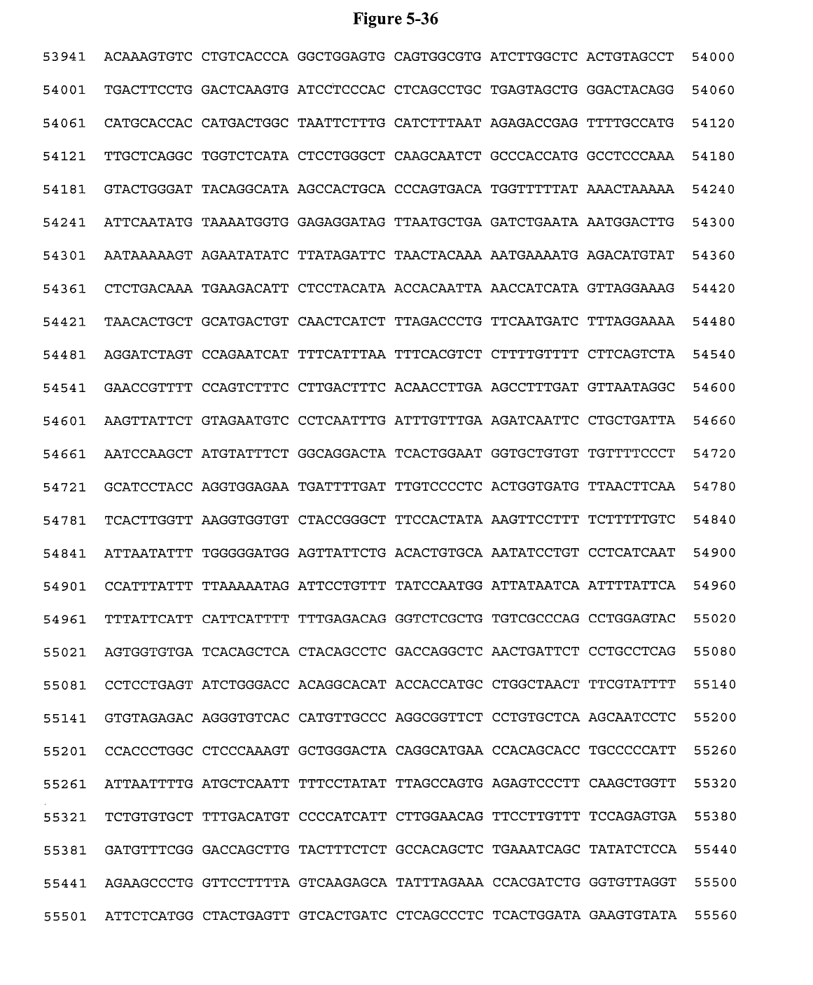

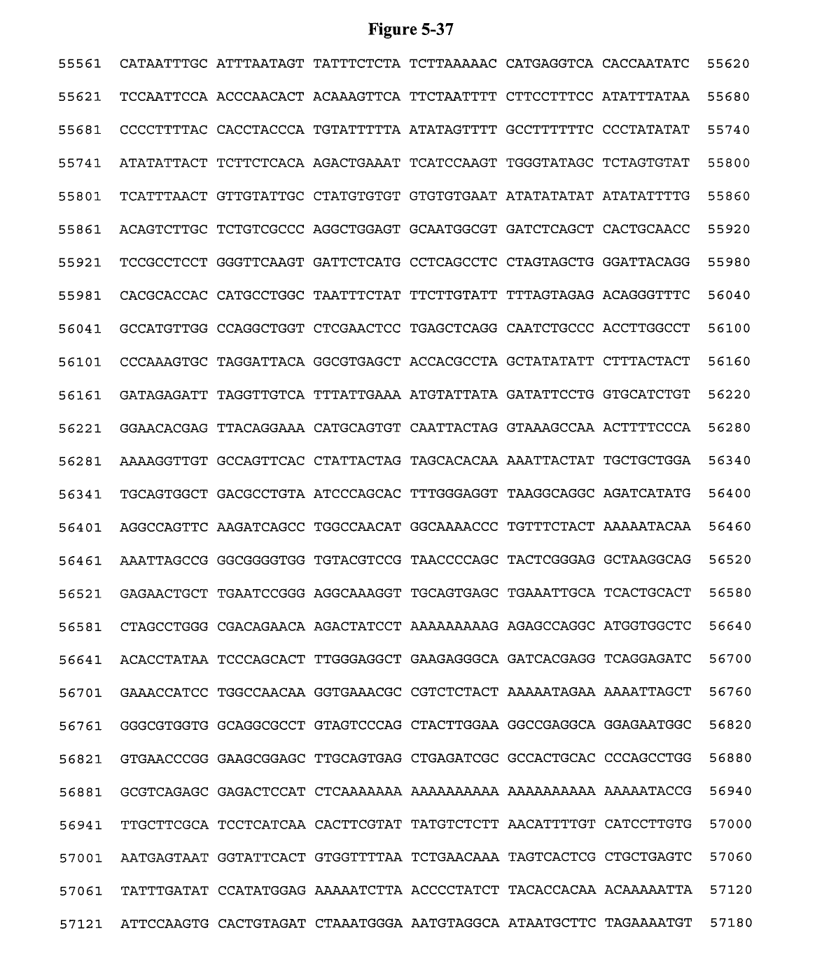

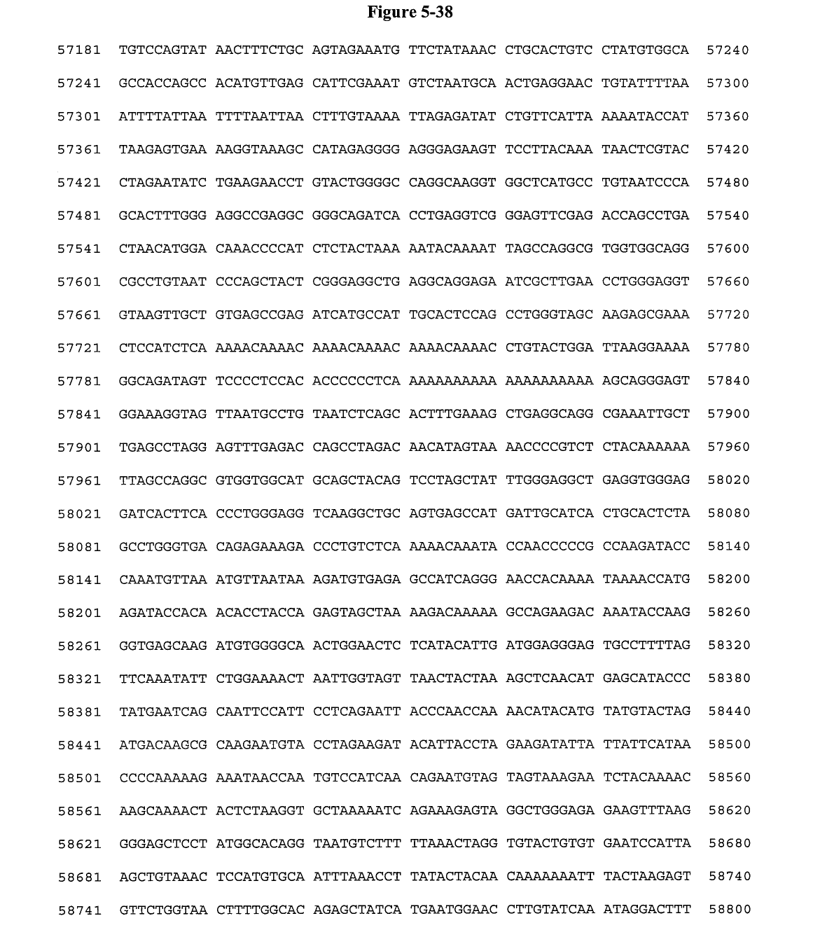

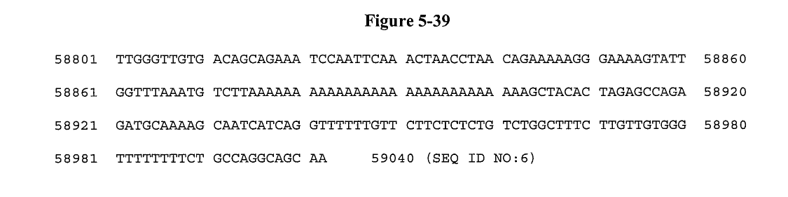

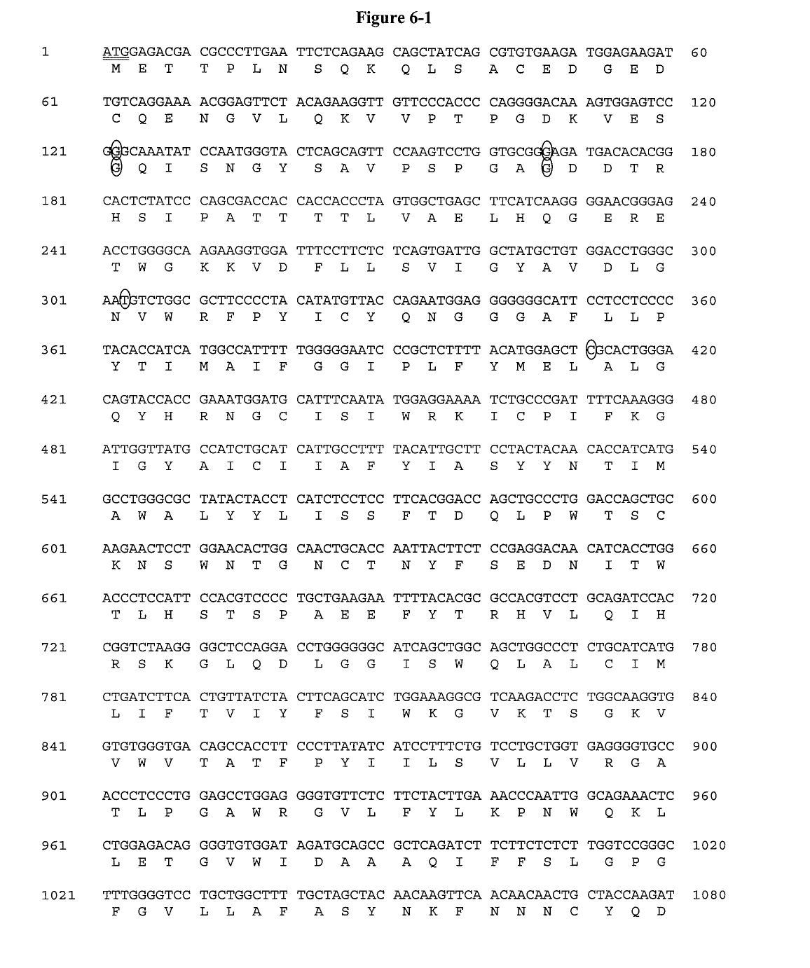

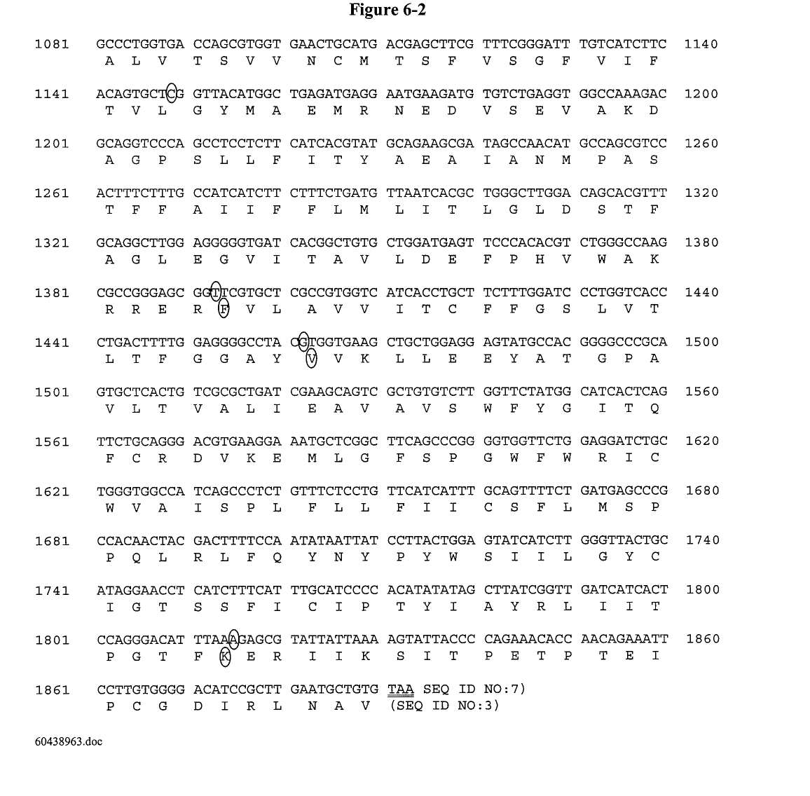

As used herein, “nucleotide sequence variant” refers to any alteration in an SLC6A4 reference sequence, and includes variations that occur in coding and non-coding regions, including exons, introns, and untranslated sequences. Nucleotides are referred to herein by the standard one-letter designation (A, C, G, or T). Variations include single nucleotide substitutions, deletions of one or more nucleotides, and insertions of one or more nucleotides. Exemplary reference SLC6A4 sequences are provided in FIGS. 1, 2, 5, and 6 (SEQ ID NOS:1, 2, 6, and 7, respectively) and also can be found in GenBank® (see, e.g., Accession Nos. U79746 and NM_{—}001045). A reference SLC6A4 amino acid sequence (SEQ ID NO:3) is shown in FIGS. 3, 5, and 6, and also can be found in GenBank® Accession No. NM_{—}001045. SLC6A4 nucleic acid and amino acid reference sequences also are referred to herein as “wild type.”

As used herein, “untranslated sequence” includes 5′ and 3′ flanking regions that are outside of the messenger RNA (mRNA) as well as 5′ and 3′ untranslated regions (5′-UTR or 3′-UTR) that are part of the mRNA, but are not translated. Positions of nucleotide sequence variants in 5′ untranslated sequences can be designated as “−X” relative to the “A” in the translation initiation codon; positions of nucleotide sequence variants in the coding sequence and 3′ untranslated sequence can be designated as “+X” or “X” relative to the “A” in the translation initiation codon. Nucleotide sequence variants that occur in introns can be designated as “+X” or “X” relative to the “G” in the splice donor site (GT) or as “−X” relative to the “G” in the splice acceptor site (AG).

In some embodiments, an SLC6A4 nucleotide sequence variant is located within an exon (e.g., exon 2, exon 10, exon 1, or exon 13). Such variants may encode an SLC6A4 polypeptide having an altered amino acid sequence. For example, a variant can contain a cytosine substitution at position 122 within exon 2 (position 24953 of SEQ ID NO:6) and encode an SLC6A4 polypeptide having an alanine substituted for glycine at position 41, a cytosine substitution at position 167 within exon 2 (position 24998 of SEQ ID NO; 6) and encode an SLC6A4 polypeptide having an alanine substituted for glycine at position 56, a cytosine substitution at position 1393 within exon 10 (position 36219 of SEQ ID NO:6) and encode an SLC6A4 polypeptide having a leucine substituted for phenylalanine at position 465, an adenine substitution at position 1462 within exon 11 (position 37560 of SEQ ID NO:6) and encode an SLC6A4 polypeptide having a methionine substituted for valine at position 488, a cytosine substitution at position 1815 within exon 13 (position 43615 of SEQ ID NO:6) and encode an SLC6A4 polypeptide having an asparagine substituted for lysine at position 605, or any combination thereof.

The term “polypeptide” refers to a chain of at least four amino acid residues (e.g., 4-8, 9-12, 13-15, 16-18, 19-21, 22-50, 50-100, 100-150, 150-200, 200-250, 250-300, 300-350 residues, or a full-length SLC6A4 polypeptide). SLC6A4 polypeptides containing amino acid sequence variants relative to an SLC6A4 reference sequence may or may not have serotonin transporter activity, or may have altered activity relative to the reference SLC6A4 polypeptide. Polypeptides that do not have activity or have altered activity can be useful for diagnostic purposes (e.g., for producing antibodies having specific binding affinity for variant SLC6A4 polypeptides).

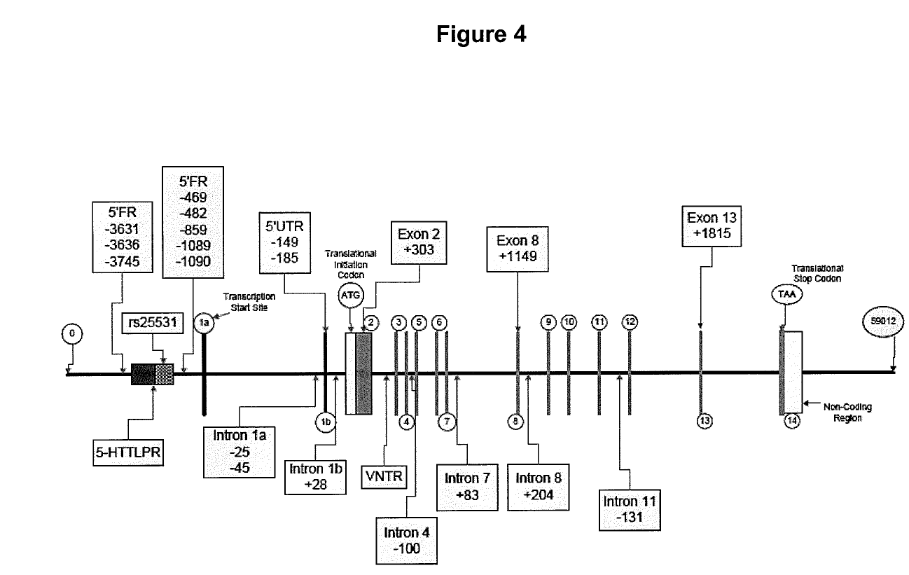

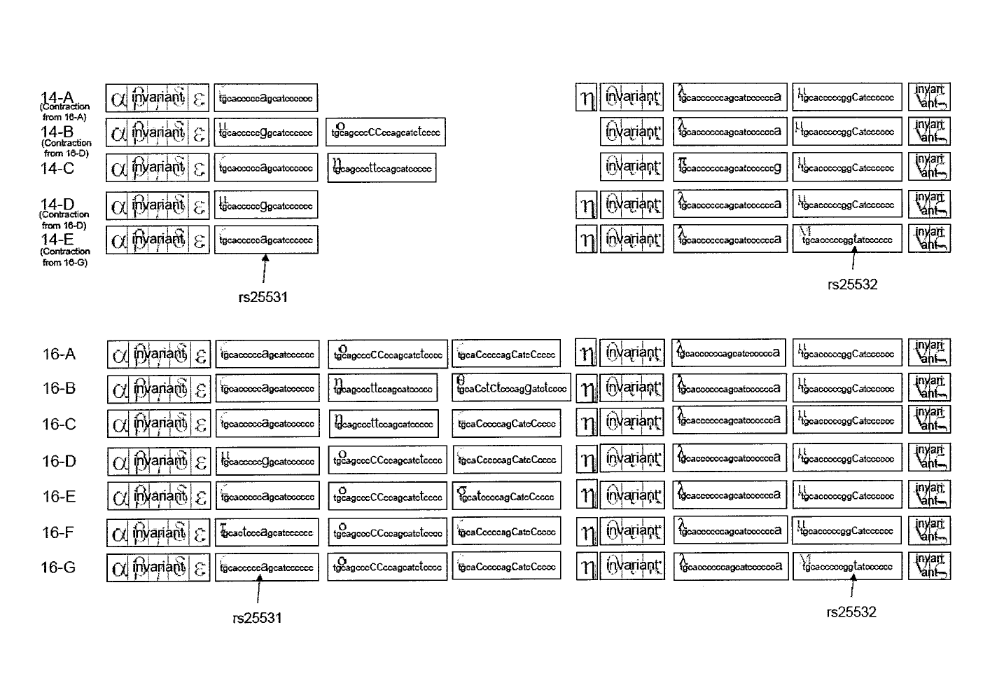

Corresponding SLC6A4 polypeptides, irrespective of length, that differ in amino acid sequence are herein referred to as allozymes. SLC6A4 allozymes are encoded by a series of SLC6A4 alleles. These alleles represent nucleic acid sequences containing sequence variants, typically multiple sequence variants, within coding and non-coding sequences. Representative examples of single nucleotide variants are described herein. For example, Table 1 sets out a series of SLC6A4 alleles that encode SLC6A4. The locations of these polymorphisms within the SLC6A4 gene are depicted in FIG. 4. Table 3 also sets out a series of SLC6A4 alleles encoding SLC6A4. Some alleles may be commonly observed, i.e., have allele frequencies >1% (e.g., >2%, >3%, >5%, >10%, or >20%), as indicated in Table 3. The number of alleles for SLC6A4 indicates the potential complexity of SLC6A4 pharmacogenetics. Such complexity emphasizes the need for determining single nucleotide variants, (i.e., single nucleotide polymorphisms, SNPs) as well as complete SLC6A4 haplotypes (i.e., the set of alleles on one chromosome or a part of a chromosome) of patients.

Certain SLC6A4 nucleotide sequence variants do not alter the amino acid sequence. Such variants, however, could alter regulation of transcription as well as mRNA stability. SLC6A4 variants that do not alter the amino acid sequence can occur in exon sequences, including, for example, exon 2, exon 3, and exon 8. For example, a nucleotide sequence variant can be a cytosine substitution at nucleotide 303 within exon 2 (position 25134 of SEQ ID NO:6), a thymine substitution at nucleotide 411 within exon 3 (position 27926 of SEQ ID NO:6), or a thymine substitution at position 1149 within exon 8 (position 33995 of SEQ ID NO:6).

SLC644 variants that do not alter the amino acid sequence also can occur in intron sequences, for example, within introns 1a, 1b, 2, 4, 8, or 11. In particular, a nucleotide sequence variant can include a cytosine substitution at position −47 of intron 1a (position 23828 of SEQ ID NO:6), an adenine substitution at nucleotide −45 of intron 1a (position 23830 of SEQ ID NO:6), an adenine substitution at nucleotide −25 of intron 1a (position 23850 of SEQ ID NO:6), an adenine substitution at nucleotide 28 of intron 1b (position 23999 of SEQ ID NO:6), a thymine substitution at nucleotide 112 of intron 1b (position 24083 of SEQ ID NO:6), an adenine substitution at nucleotide 162 of intron 1b (position 24133 of SEQ ID NO:6), a cytosine substitution at nucleotide 242 of intron 1b (position 24213 of SEQ ID NO:6), insertion or deletion of tandem repeats (VNTR) within intron 2 (at positions 25201 to 25403 of SEQ ID NO:6), a thymine substitution at nucleotide 11 of intron 3 (position 28004 of SEQ ID NO:6), a cytosine substitution at nucleotide −105 of intron 3 (position 28348 of SEQ ID NO:6), a thymine substitution at nucleotide 10 of intron 4 (position 28682 of SEQ ID NO:6), an adenine substitution at nucleotide −100 of intron 4 (position 29386 of SEQ ID NO:6), a thymine substitution at nucleotide −86 of intron 4 (position 29400 of SEQ ID NO:6), an adenine substitution at nucleotide −85 of intron 4 (position 29401 of SEQ ID NO:6), an adenine substitution at nucleotide 56 of intron 6 (position 30764 of SEQ ID NO:6), a cytosine substitution at nucleotide 67 of intron 6 (position 30775 of SEQ ID NO:6), a thymine substitution at nucleotide −115 of intron 6 (position 30961 of SEQ ID NO:6), a cytosine substitution at nucleotide −44 of intron 6 (position 31032 of SEQ ID NO:6), a thymine substitution at nucleotide −37 of intron 6 (position 31039 of SEQ ID NO:6), a thymine substitution at nucleotide 83 of intron 7 (position 31262 of SEQ ID NO:6), an adenine substitution at nucleotide −24 of intron 7 (position 33899 of SEQ ID NO:6), a guanine substitution at nucleotide 204 of intron 8 (position 34254 of SEQ ID NO:6), a thymine substitution at nucleotide 220 of intron 8 (position 34270 of SEQ ID NO:6), an adenine substitution at nucleotide −25 of intron 9 (position 36119 of SEQ ID NO:6), a thymine substitution at nucleotide 69 of intron 10 (position 36344 of SEQ ID NO:6), a guanine substitution at nucleotide −40 of intron 10 (position 37508 of SEQ ID NO:6), a thymine substitution at nucleotide −131 of intron 11 (position 38827 of SEQ ID NO:6), or a thymine substitution at nucleotide −118 of intron 11 (position 38840 of SEQ ID NO:6).

SLC6A4 nucleotide sequence variants that do not change the amino acid sequence also can be within 5′ or 3′ untranslated sequences. For example, with respect to the adenine in the translation initiation codon, a nucleotide sequence variant can be a thymine substitution at nucleotide −3791 (position 7607 of SEQ ID NO:6), a cytosine substitution at nucleotide −3780 (position 7618 of SEQ ID NO:6), an adenine substitution at nucleotide −3745 (position 7653 of SEQ ID NO:6), a thymine substitution at nucleotide −3684 (position 7714 of SEQ ID NO:6), a cytosine substitution at nucleotide −3636 (position 7762 of SEQ ID NO:6), an adenine substitution at nucleotide −3631 (position 7767 of SEQ ID NO:6), a 44 bp deletion between nucleotides −2063 and −1714 (positions 9335 to 9684 of SEQ ID NO:6), a guanine substitution at the rs25531 allele, a thymine at nucleotide −1090 (position 10308 of SEQ ID NO:6), a thymine substitution at nucleotide −1089 (position 10309 of SEQ ID NO:6), a cytosine substitution at nucleotide −859 (position 10539 of SEQ ID NO:6), an adenine substitution at nucleotide −650 (position 10748 of SEQ ID NO:6), a cytosine substitution at nucleotide −482 (position 10916 of SEQ ID NO:6), a thymine substitution at nucleotide −469 position 10929 of SEQ ID NO:6), a cytosine substitution at nucleotide −185 (position 23910 of SEQ ID NO:6), or an adenine substitution at nucleotide −149 (position 23946 of SEQ ID NO:6).

TABLE 1
SLC6A4 polymorphisms

| | | Sequence |
|---|---|---|
| Location | Nucleotide(s) | Change |
| 5′FR | −3745 | T→A |
| 5′FR | −3636 | T→C |
| 5′FR | −3631 | G→A |
| 5′FR | −2063 to −1714 | 44 bp deletion |
| | | (HTTLPR short) |
| 5′FR | SNP rs25531 | A→G |
| 5′FR | −1090 | A→T |
| 5′FR | −1089 | A→T |
| 5′FR | −859 | A→C |
| 5′FR | −482 | T→C |
| 5′FR | −469 | C→T |
| Intron 1a | −45 | C→A |
| Intron 1a | −25 | G→A |
| 5′UTR | −185 | A→C |
| 5′UTR | −149 | C→A |
| Intron 1b | 28 | G→A |
| Exon 2 | 303 | T→C |
| Intron 2 | | VNTR-9 |
| Intron 2 | | VNTR-10 |
| Intron 2 | | VNTR-11 |
| Intron 2 | | VNTR-12 |
| Intron 4 | −100 | G→A |
| Intron 7 | 83 | C→T |
| Exon 8 | 1149 | C→T |
| Intron 8 | 204 | T→G |
| Intron 11 | −131 | C→T |
| Exon 13 | 1815 | A→C |
| | SNP rs25532 | C→T |

In some embodiments, an SLC6A4 nucleic acid molecule can consist essentially of at least ten (e.g., at least 12, at least 15, at least 18, at least 20, or at least 25) contiguous nucleotides of an SLC6A4 reference sequence (e.g., SEQ ID NO:1, SEQ ID NO:2, or SEQ ID NO:6). Such nucleic acids can contain one or more variant positions, with the proviso that the nucleotides at those positions are variant nucleotides as disclosed herein in Table 1 or Table 3, for example. An SLC6A4 nucleic acid “consisting essentially of” a particular sequence has the basic and novel characteristic that it can be used to distinguish, based upon hybridization, a nucleic acid having a sequence that contains a variant from a corresponding nucleic acid having a sequence that does not contain the variant (e.g., a wild type sequence). Such nucleic acid molecules can include one or more additional sequences (e.g., a restriction sequence and/or a sequence encoding a tag such as polyhistidine, GFP, or any other suitable tag), or can include one or more labels (e.g., biotin or a fluorescent label as disclosed herein), provided that such additions do not affect the basic and novel characteristic of the nucleic acid molecules.

In some embodiments, an SLC6A4 nucleic acid sequence can have at least 90% (e.g., 90%, 91%, 92%, 93%, 94%, 95%, 96%, 97%, 97.5%, 98%, 98.5%, 99.0%, 99.5%, 99.6%, 99.7%, 99.8%, 99.9%, or 100%) sequence identity with a region of a reference SLC6A4 sequence (e.g., SEQ ID NO:1). The region of the reference sequence can be at least ten nucleotides in length (e.g., 10, 15, 20, 50, 60, 70, 75, 100, 150 or more nucleotides in length).

Percent sequence identity is calculated by determining the number of matched positions in aligned nucleic acid sequences, dividing the number of matched positions by the total number of aligned nucleotides, and multiplying by 100. A matched position refers to a position in which identical nucleotides occur at the same position in aligned nucleic acid sequences. Percent sequence identity also can be determined for any amino acid sequence. To determine percent sequence identity, a target nucleic acid or amino acid sequence is compared to the identified nucleic acid or amino acid sequence using the BLAST 2 Sequences (B12seq) program from the stand-alone version of BLASTZ containing BLASTN version 2.0.14 and BLASTP version 2.0.14. This stand-alone version of BLASTZ can be obtained on the World Wide Web from Fish & Richardson's web site (fr.com/blast) or the U.S. government's National Center for Biotechnology Information web site (ncbi.nlm.nih.gov). Instructions explaining how to use the B12seq program can be found in the readme file accompanying BLASTZ.

B12seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: −i is set to a file containing the first nucleic acid sequence to be compared (e.g., C:\seq1.txt); −j is set to a file containing the second nucleic acid sequence to be compared (e.g., C:\seq2.txt); −p is set to blastn; −o is set to any desired file name (e.g., C:\output.txt); −q is set to −1; −r is set to 2; and all other options are left at their default setting. The following command will generate an output file containing a comparison between two sequences: C:\B12seq −i c:\seq1.txt −j c:\seq2.txt −p blastn −o c:\output.txt −q −1 −r 2. If the target sequence shares homology with any portion of the identified sequence, then the designated output file will present those regions of homology as aligned sequences. If the target sequence does not share homology with any portion of the identified sequence, then the designated output file will not present aligned sequences.

Once aligned, a length is determined by counting the number of consecutive nucleotides from the target sequence presented in alignment with sequence from the identified sequence starting with any matched position and ending with any other matched position. A matched position is any position where an identical nucleotide is presented in both the target and identified sequence. Gaps presented in the target sequence are not counted since gaps are not nucleotides. Likewise, gaps presented in the identified sequence are not counted since target sequence nucleotides are counted, not nucleotides from the identified sequence.

The percent identity over a particular length is determined by counting the number of matched positions over that length and dividing that number by the length followed by multiplying the resulting value by 100. For example, if (1) a 1000 nucleotide target sequence is compared to the sequence set forth in SEQ ID NO:1, (2) the B12seq program presents 969 nucleotides from the target sequence aligned with a region of the sequence set forth in SEQ ID NO:1 where the first and last nucleotides of that 969 nucleotide region are matches, and (3) the number of matches over those 969 aligned nucleotides is 900, then the 1000 nucleotide target sequence contains a length of 969 and a percent identity over that length of 93 (i.e., 900÷969×100=93).

It will be appreciated that different regions within a single nucleic acid target sequence that aligns with an identified sequence can each have their own percent identity. It is noted that the percent identity value is rounded to the nearest tenth. For example, 78.11, 78.12, 78.13, and 78.14are rounded down to 78.1, while 78.15, 78.16, 78.17, 78.18, and 78.19 are rounded up to 78.2. It also is noted that the length value will always be an integer.

Isolated nucleic acid molecules can be produced by standard techniques, including, without limitation, common molecular cloning and chemical nucleic acid synthesis techniques. For example, polymerase chain reaction (PCR) techniques can be used to obtain an isolated nucleic acid containing an SLC6A4 nucleotide sequence variant. PCR refers to a procedure or technique in which target nucleic acids are enzymatically amplified. Sequence information from the ends of the region of interest or beyond typically is employed to design oligonucleotide primers that are identical in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize complementary DNA (cDNA) strands. Ligase chain reaction, strand displacement amplification, self-sustained sequence replication, or nucleic acid sequence-based amplification also can be used to obtain isolated nucleic acids. See, for example, Lewis (1992) Genetic Engineering News 12(9):1; Guatelli et al. (1998) Proc. Natl. Acad. Sci. USA 87.1874-1878; and Weiss (1991) Science 254:1292.

Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector.

Isolated nucleic acids also can be obtained by mutagenesis. For example, the reference sequences depicted in FIGS. 1, 2, and 6 can be mutated using standard techniques including oligonucleotide-directed mutagenesis and site-directed mutagenesis through PCR. See, Short Protocols in Molecular Biology, Chapter 8, Green Publishing Associates and John Wiley & Sons, edited by Ausubel et al., 1992. Examples of positions that can be modified include those described herein.

SLC6A4 Polypeptides

The isolated SLC6A4 polypeptides provided herein can include an amino acid sequence variant relative to a reference SLC6A4 sequence as set forth, for example, in FIGS. 3, 5, and 6 (SEQ ID NO:3; GenBank® Accession No. NM_{—}001045). The term “isolated” with respect to an SLC6A4 polypeptide refers to a polypeptide that has been separated from cellular components by which it is naturally accompanied. Typically, the polypeptide is isolated when it is at least 60% (e.g., 70%, 80%, 90%, 95%, or 99%), by weight, free from proteins and naturally-occurring organic molecules with which it is naturally associated. In general, an isolated polypeptide will yield a single major band on a non-reducing polyacrylamide gel.

An SLC6A4 polypeptide can include a variant at one or more amino acid with respect to the reference sequence. In some embodiments, the serotonin transporter activity of a variant SLC6A4 polypeptide can be altered relative to the reference SLC6A4. Certain SLC6A4 allozymes can have reduced activity, while other allozymes can have activity that is comparable to the reference SLC6A4. Other allozymes can have increased activity relative to the reference SLC6A4. Activity of SLC6A4 polypeptides can be assessed in vitro. For example, the methods described by Kendall et al. (J. Biol. Chem. (1998) 273:28098-28106) can be used. Briefly, [³H]serotonin transport assays can be carried out in cells transfected with an SLC6A4 expression vector. For example, transport can be measured by incubating cells with [1,2-³H]serotonin in PBS containing CaCl₂ and MgCl₂, washing and lysing the cells, and counting the lysates to evaluate the ³H levels. Other biochemical properties of allozymes, such as apparent Kₘ values, also can be altered relative to the reference SLC6A4.

Isolated polypeptides can be obtained, for example, by extraction from a natural source (e.g., mammary gland tissue), chemical synthesis, or by recombinant production in a host cell. To recombinantly produce SLC6A4 polypeptides, a nucleic acid encoding an SLC6A4 nucleotide sequence variant can be ligated into an expression vector and used to transform a prokaryotic (e.g., bacteria) or eukaryotic (e.g., insect, yeast, or mammal) host cell. In general, nucleic acid constructs include a regulatory sequence operably linked to an SLC6A4 nucleic acid sequence. Regulatory sequences (e.g., promoters, enhancers, polyadenylation signals, or terminators) do not typically encode a gene product, but instead affect the expression of the nucleic acid sequence. In addition, a construct can include a tag sequence designed to facilitate subsequent manipulations of the expressed nucleic acid sequence (e.g., purification, localization). Tag sequences, such as green fluorescent protein (GFP), glutathione S-transferase (GST), six histidine (His₆), c-myc, hemagglutinin, or Flag™ tag (Kodak) sequences are typically expressed as a fusion with the expressed nucleic acid sequence. Such tags can be inserted anywhere within the polypeptide including at either the carboxyl or amino termini. The type and combination of regulatory and tag sequences can vary with each particular host, cloning or expression system, and desired outcome. A variety of cloning and expression vectors containing combinations of regulatory and tag sequences are commercially available. Suitable cloning vectors include, without limitation, pUC18, pUC19, and pBR322 and derivatives thereof (New England Biolabs, Beverly, Mass.), and pGEN (Promega, Madison, Wis.). Additionally, representative prokaryotic expression vectors include pBAD (Invitrogen, Carlsbad, Calif.), the pTYB family of vectors (New England Biolabs), and pGEMEX vectors (Promega); representative mammalian expression vectors include pTet-On/pTet-Off (Clontech, Palo Alto, Calif.), pIND, pVAX1, pCR3.1, pcDNA3.1, pcDNA4, or pUni (Invitrogen), and pCI or pSI (Promega); representative insect expression vectors include pBacPAK8 or pBacPAK9 (Clontech), and p2Bac (Invitrogen); and representative yeast expression vectors include MATCHMAKER (Clontech) and pPICZ A, B, and C (Invitrogen).

In bacterial systems, a strain of Escherichia coli can be used to express SLC6A4 variant polypeptides. For example, BL-21 cells can be transformed with a pGEX vector containing an SLC6A4 nucleic acid sequence. The transformed bacteria can be grown exponentially and then stimulated with isopropylthiogalactopyranoside (IPTG) prior to harvesting. In general, the SLC6A4-GST fusion proteins produced from the pGEX expression vector are soluble and can be purified easily from lysed cells by adsorption to glutathione-agarose beads followed by elution in the presence of free glutathione. The pGEX vectors are designed to include thrombin or factor Xa protease cleavage sites so that the expressed SLC6A4 polypeptide can be released from the GST moiety.

In eukaryotic host cells, a number of viral-based expression systems can be utilized to express SLC6A4 variant polypeptides. A nucleic acid encoding a polypeptide can be cloned into, for example, a baculoviral vector such as pBlueBac (Invitrogen) and then used to co-transfect insect cells such as Spodoptera frugiperda (Sf9) cells with wild type DNA from Autographa californica multinuclear polyhedrosis virus (AcMNPV). Recombinant viruses producing polypeptides can be identified by standard methodology. Alternatively, a nucleic acid encoding a polypeptide can be introduced into a SV40, retroviral, or vaccinia based viral vector and used to infect suitable host cells.

Eukaryotic cell lines that stably express SLC6A4 variant polypeptides can be produced using expression vectors with the appropriate control elements and a selectable marker. For example, the eukaryotic expression vector pCR3.1 (Invitrogen, San Diego, Calif.) and p91023(B) (see Wong et al. (1985) Science 228:810-815) or modified derivatives thereof are suitable for expression of SLC6A4 variant polypeptides in, for example, Chinese hamster ovary (CHO) cells, COS-1 cells, human embryonic kidney 293 cells, NIH3T3 cells, BHK21 cells, MDCK cells, and human vascular endothelial cells (HUVEC). Following introduction of the expression vector by electroporation, lipofection, calcium phosphate or calcium chloride co-precipitation, DEAE dextran, or other suitable transfection method, stable cell lines are selected, e.g., by antibiotic resistance to G418, kanamycin, or hygromycin. Alternatively, amplified sequences can be ligated into a eukaryotic expression vector such as pcDNA3 (Invitrogen) and then transcribed and translated in vitro using wheat germ extract or rabbit reticulocyte lysate.

SLC6A4 variant polypeptides can be purified using known chromatographic methods including ion exchange and gel filtration chromatography. See, for example, Caine et al. (1996) Protein Expr. Purif. 8:159-166. SLC6A4 polypeptides can be “engineered” to contain a tag sequence describe herein that allows the polypeptide to be purified (e.g., captured onto an affinity matrix). Immunoaffinity chromatography also can be used to purify SLC6A4 polypeptides.

Determining Genotype

Genomic DNA generally is used to determine genotype, although mRNA also can be used. Genomic DNA is typically extracted from a biological sample such as a peripheral blood sample, but can be extracted from other biological samples, including tissues (e.g., mucosal scrapings of the lining of the mouth or from renal or hepatic tissue). Routine methods can be used to extract genomic DNA from a blood or tissue sample, including, for example, phenol extraction. Alternatively, genomic DNA can be extracted with kits such as the QIAamp® Tissue Kit (Qiagen, Chatsworth, Calif.), Wizard® Genomic DNA purification kit (Promega) and the A.S.A.P.™ Genomic DNA isolation kit (Boehringer Mannheim, Indianapolis, Ind.).

Typically, an amplification step is performed before proceeding with the genotyping. For example, polymerase chain reaction (PCR) techniques can be used to obtain amplification products from the patient. PCR refers to a procedure or technique in which target nucleic acids are enzymatically amplified. Sequence information from the ends of the region of interest or beyond typically is employed to design oligonucleotide primers that are identical in sequence to opposite strands of the template to be amplified. PCR can be used to amplify specific sequences from DNA as well as RNA, including sequences from total genomic DNA or total cellular RNA. Primers are typically 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize complementary DNA (cDNA) strands. Ligase chain reaction, strand displacement amplification, self-sustained sequence replication or nucleic acid sequence-based amplification also can be used to obtain isolated nucleic acids. See, for example, Lewis (1992) Genetic Engineering News 12(9):1; Guatelli et al. (1990) Proc. Natl. Acad. Sci. USA 87:1874-1878; and Weiss (1991) Science 254:1292-1293.

Primers typically are single-stranded or double-stranded oligonucleotides that are 10 to 50 nucleotides in length, and when combined with mammalian genomic DNA and subjected to PCR conditions, is capable of being extended to produce a nucleic acid product corresponding to a region of interest within a gene. Typically, PCR products are at least 30 nucleotides in length (e.g., 30, 35, 50, 100, 250, 500, 1000, 1500, or 2000 or more nucleotides in length). Specific regions of mammalian DNA can be amplified (i.e., replicated such that multiple exact copies are produced) when a pair of oligonucleotide primers is used in the same PCR reaction, wherein one primer contains a nucleotide sequence from the coding strand of a nucleic acid and the other primer contains a nucleotide sequence from the non-coding strand of the nucleic acid. The “coding strand” of a nucleic acid is the nontranscribed strand, which has the same nucleotide sequence as the specified RNA transcript (with the exception that the RNA transcript contains uracil in place of thymidine residues), while the “non-coding strand” of a nucleic acid is the strand that serves as the template for transcription.

A single PCR reaction mixture may contain one pair of oligonucleotide primers. Alternatively, a single reaction mixture may contain a plurality of oligonucleotide primer pairs, in which case multiple PCR products can be generated (e.g., 5, 10, 15, or 20 primer pairs). Each primer pair can amplify, for example, one exon or a portion of one exon. Intron sequences also can be amplified.

Exons or introns of a gene of interest can be amplified then directly sequenced. Dye primer sequencing can be used to increase the accuracy of detecting heterozygous samples. Alternatively, one or more of the techniques described below can be used to determine genotype.

Nucleic acid molecules provided herein can be used to detect variant SLC6A4 sequences. For example, allele specific hybridization can be used to detect sequence variants, including complete haplotypes and genotypes of a mammal. See, Stoneking et al. (1991) Am. J. Hum. Genet. 48:370-382; and Prince et al. (2001) Genome Res. 11:152-162. Examples of genotypes for the long and short alleles in combination with the rs25531 polymorphism, as ascertained for several race groups, are shown in Table 2. Other genotypes can include additional or alternate alleles. For example, a subject can have a long allele with an rs25531a allele and an rs25532t allele. Alternatively or in addition, a subject can have a short allele with an rs25531a allele and an rs25532t allele. In practice, samples of DNA or RNA from one or more mammals can be amplified using pairs of primers and the resulting amplification products can be immobilized on a substrate (e.g., in discrete regions). Hybridization conditions are selected such that a nucleic acid probe can specifically bind to the sequence of interest, e.g., the variant nucleic acid sequence. Such hybridizations typically are performed under high stringency as some sequence variants include only a single nucleotide difference. High stringency conditions can include the use of low ionic strength solutions and high temperatures for washing. For example, nucleic acid molecules can be hybridized at 42° C. in 2×SSC (0.3M NaCl/0.03 M sodium citrate/0.1% sodium dodecyl sulfate (SDS) and washed in 0.1×SSC (0.015M NaCl/0.0015 M sodium citrate), 0.1% SDS at 65° C. Hybridization conditions can be adjusted to account for unique features of the nucleic acid molecule, including length and sequence composition. Probes can be labeled (e.g., fluorescently) to facilitate detection. In some embodiments, one of the primers used in the amplification reaction is biotinylated (e.g., 5′ end of reverse primer) and the resulting biotinylated amplification product is immobilized on an avidin or streptavidin coated substrate (e.g., in discrete regions).

Allele-specific restriction digests can be performed in the following manner. For nucleotide sequence variants that introduce a restriction site, restriction digest with the particular restriction enzyme can differentiate the alleles. For sequence variants that do not alter a common restriction site, mutagenic primers can be designed that introduce a restriction site when the variant allele is present or when the wild type allele is present. A portion of the nucleic acid of interest can be amplified using the mutagenic primer and a wild type primer, followed by digest with the appropriate restriction endonuclease.

Certain variants, such as insertions or deletions of one or more nucleotides, change the size of the DNA fragment encompassing the variant. The insertion or deletion of nucleotides can be assessed by amplifying the region encompassing the variant and determining the size of the amplified products in comparison with size standards. For example, a region of a gene of interest can be amplified using a primer set from either side of the variant. One of the primers is typically labeled, for example, with a fluorescent moiety, to facilitate sizing. The amplified products can be electrophoresed through acrylamide gels with a set of size standards that are labeled with a fluorescent moiety that differs from the primer.

PCR conditions and primers can be developed that amplify a product only when the variant allele is present or only when the wild type allele is present (MSPCR or allele-specific PCR). For example, patient DNA and a control can be amplified separately using either a wild type primer or a primer specific for the variant allele. Each set of reactions is then examined for the presence of amplification products using standard methods to visualize the DNA. For example, the reactions can be electrophoresed through an agarose gel and the DNA visualized by staining with ethidium bromide or other DNA intercalating dye. In DNA samples from heterozygous patients, reaction products would be detected in each reaction. Patient samples containing solely the wild type allele would have amplification products only in the reaction using the wild type primer. Similarly, patient samples containing solely the variant allele would have amplification products only in the reaction using the variant primer. Allele-specific PCR also can be performed using allele-specific primers that introduce priming sites for two universal energy-transfer-labeled primers (e.g., one primer labeled with a green dye such as fluoroscein and one primer labeled with a red dye such as sulforhodamine). Amplification products can be analyzed for green and red fluorescence in a plate reader. See, Myakishev et al. (2001) Genome 11:163-169.

Mismatch cleavage methods also can be used to detect differing sequences by PCR amplification, followed by hybridization with the wild type sequence and cleavage at points of mismatch. Chemical reagents, such as carbodiimide or hydroxylamine and osmium tetroxide can be used to modify mismatched nucleotides to facilitate cleavage.

TABLE 2
Genotyping for 5HTTLPR and SNP rs25531

| Predicted length/SNP* | | AA** | WH | WNH | Other | Totals |
|---|---|---|---|---|---|---|
| La/La | N | 88 | 50 | 329 | 30 | 497 |
| | % of Total | 4.63 | 2.63 | 17.29 | 1.58 | 26.13 |
| La/Lg | N | 83 | 12 | 99 | 9 | 203 |
| | % of Total | 4.36 | 0.63 | 5.21 | 0.47 | 10.67 |
| La/Sa | N | 64 | 98 | 517 | 37 | 716 |
| | % of Total | 3.36 | 5.15 | 27.18 | 1.95 | 37.64 |
| Lg/Lg | N | 15 | 3 | 6 | 0 | 24 |
| | % of Total | 0.79 | 0.16 | 0.31 | 0 | 1.26 |
| Lg/Sa | N | 31 | 11 | 72 | 12 | 126 |
| (Unverified) | | | | | | |
| | % of Total | 1.63 | 0.58 | 3.79 | 0.63 | 6.63 |
| Sa/Sa | N | 14 | 69 | 225 | 24 | 332 |
| | % of Total | 0.74 | 3.63 | 11.83 | 1.26 | 17.46 |
| Sa/Sg | N | 0 | 0 | 4 | 0 | 4 |
| | % of Total | 0 | 0 | 0.21 | 0 | 0.21 |
| | Totals (N) | 295 | 243 | 1252 | 112 | 1902 |
| | (%) | 15.51 | 12.78 | 65.82 | 5.89 | 100 |

*L = long allele; S = short allele; a = rs25531a allele; g = rs25531g allele
**AA = African American; WH = white Hispanic; WNH = white non-Hispanic

Serotonin Transporter Status

Given the disclosure provided herein, it is possible to determine serotonin transport status of a subject (e.g., a mammal such as a human). “Serotonin transport status” refers to the ability of a subject to transport serotonin from synaptic spaces into presynaptic neurons. Serotonin transport status of a subject can be determined by, for example, measuring the level of SLC6A4 activity in the subject using, for example, the methods described herein. Alternatively, serotonin transport status can be evaluated by determining whether an SLC6A4 nucleic acid sequence of a subject contains one or more variants (e.g., one or more variants that are correlated with increased or decreased SLC6A4 activity). A variant that results in decreased or increased SLC6A4 activity can be said to result in “reduced” or “enhanced” serotonin transport status, respectively. In some embodiments, the variant profile of a subject can be used to determine the serotonin transport status of the subject.

“Variant profile” refers to the presence or absence of a plurality (e.g., two or more) of SLC6A4 nucleotide sequence variants or SLC6A4 amino acid sequence variants. For example, a variant profile can include the complete SLC6A4 haplotype of the mammal, or can include the presence or absence of a set of particular non-synonymous SNPs (e.g., single nucleotide substitutions that alter the amino acid sequence of an SLC6A4 polypeptide). In one embodiment, the variant profile includes detecting the presence or absence of two or more non-synonymous SNPs (e.g., 2, 3, or 4 non-synonymous SNPs) described herein. There may be ethnic-specific pharmacogenetic variation, as certain nucleotide and amino acid sequence variants may be detected solely in African-American, Caucasian-American, Han Chinese-American, or Mexican-American subjects. In addition, the variant profile can include detecting the presence or absence of any type of SLC6A4 SNP together with any other SLC6A4 SNP (e.g., a polymorphism pair or a group of polymorphism pairs). Further, a variant profile can include detecting the presence or absence of any SLC6A4 SNP together with any SNP from one or more other genes involved in uptake and/or metabolism of one or more psychotropic drugs (e.g., antidepressants or mood elevating agents such as selective serotonin-reuptake inhibitors (SSRIs), including fluoxetine, fluvoxamine, paroxetine, sertraline, citalopram, escitalopram, and venlafaxine, and noradrenergic and specific serotonergic antidepressants such as mirtazapine).

Serotonin transporter activity of SLC6A4 can be measured using, for example, in vitro methods such as those described herein. As used herein, the term “reduced serotonin transporter status” refers to a decrease (e.g., a 5%, 10%, 15%, 20%, 25%, 30%, 40%, 50%, 60%, 70%, 75%, 80%, 90%, 95%, or 100% decrease) in serotonin transporter activity (e.g., SLC6A4 activity) of a subject, as compared to a control level of serotonin transporter activity. Similarly, the term “enhanced serotonin transporter status” refers to an increase (e.g., a 5%, 10%, 15%, 20%, 25%, 30%, 40%, 50%, 60%, 70%, 75%, 80%, 90%, 95%, 100%, or more than 100% increase) in serotonin transporter activity of a subject, as compared to a control level of serotonin transporter activity. A control level of serotonin transporter activity can be, for example, an average level of serotonin transporter activity in a population of individuals. In one embodiment, the population includes individuals that do not contain particular SLC6A4 nucleotide sequence variants or particular SLC6A4 amino acid sequence variants (e.g., particular variants that affect serotonin transporter status). Alternatively, a control level of serotonin transporter activity can refer to the level of serotonin transporter activity in a control subject (e.g., a subject that does not contain an SLC6A4 nucleic acid containing a variant).

In some embodiments, evaluation of serotonin transporter status can be used in diagnostic assays. In further embodiments, serotonin transporter status can be linked to predisposition to a particular condition. Alterations in serotonin transport may lead to altered neurotransmitter levels, and may play a role in sudden infant death syndrome, aggressive behavior in Alzheimer disease patients, and depression-susceptibility in people experiencing emotional trauma. Predisposition to such a condition can be determined based on the presence or absence of a single SLC6A4 sequence variant or based on a variant profile.

Determination of serotonin transporter status and predisposition to particular conditions can include identification of genetic markers (e.g., polymorphisms) in linkage disequilibrium with particular SLC6A4 alleles. Although such markers may not be relevant from a functional perspective (i.e., may not directly affect function of SLC6A4), their presence can be predictive of functional/clinically relevant polymorphisms. Thus, this document also provides methods for detecting a genotype by screening for genetic markers in linkage disequilibrium with particular SLC6A4 alleles. The methods can include providing a nucleic acid sample from a subject (e.g., a human subject), and screening the sample for one or more markers in linkage disequilibrium with a particular SLC6A4 allele. For example, a method can include screening for markers in linkage disequilibrium with a SNP at nucleotide −3745, −3636, −3631, −1090, −1089, −859, −482, −469, −185, or −149 relative to the adenine in the translation initiation codon. Methods also can include screening for genetic markers in linkage disequilibrium with any of the other SNPs shown in Table 1.

Articles of Manufacture

Also provided herein are articles of manufacture, which can include populations of isolated SLC6A4 nucleic acid molecules or SLC6A4 polypeptides immobilized on a substrate. Suitable substrates provide a base for the immobilization of the nucleic acids or polypeptides, and in some embodiments, allow immobilization of nucleic acids or polypeptides into discrete regions. In embodiments in which the substrate includes a plurality of discrete regions, different populations of isolated nucleic acids or polypeptides can be immobilized in each discrete region. Thus, each discrete region of the substrate can include a different SLC6A4 nucleic acid or SLC6A4 polypeptide sequence variant. Such articles of manufacture can include two or more sequence variants of SLC6A4, or can include all of the sequence variants known for SLC6A4. For example, the article of manufacture can include two or more of the sequence variants identified herein and one or more other SLC6A4 sequence variants, such as nucleic acid variants that occur in the promoter region of the SLC6A4 gene.

Suitable substrates can be of any shape or form and can be constructed from, for example, glass, silicon, metal, plastic, cellulose, or a composite. For example, a suitable substrate can include a multiwell plate or membrane, a glass slide, a chip, or polystyrene or magnetic beads. Nucleic acid molecules or polypeptides can be synthesized in situ, immobilized directly on the substrate, or immobilized via a linker, including by covalent, ionic, or physical linkage. Linkers for immobilizing nucleic acids and polypeptides, including reversible or cleavable linkers, are known in the art. See, for example, U.S. Pat. No. 5,451,683 and WO98/20019. Immobilized nucleic acid molecules are typically about 20 nucleotides in length, but can vary from about 10 nucleotides to about 1000 nucleotides in length.

In practice, a sample of DNA or RNA from a subject can be amplified, the amplification product hybridized to an article of manufacture containing populations of isolated nucleic acid molecules in discrete regions, and hybridization can be detected. Typically, the amplified product is labeled to facilitate detection of hybridization. See, for example, Hacia et al. (1996) Nature Genet. 14:441-447; and U.S. Pat. Nos. 5,770,722 and 5,733,729.

The invention will be further described in the following examples, which do not limit the scope of the invention described in the claims.

### EXAMPLES

PCR Amplification and DNA Sequencing: DNA samples (e.g., from Caucasian-American, African-American, Han Chinese American, and/or Mexican-American subjects) were obtained from the Coriell Institute Cell Repository (Camden, N.J.). PCR reactions were performed with each DNA sample to amplify SLC6A4 exons and splice junctions. The amplicons were then sequenced using dye terminator sequencing. Universal M13 sequencing tags were added to the 5′-ends of the forward and reverse primers. For example, forward primers contained the M13 forward sequence (5′-TGTAAAACGACGGC CAGT-3′; SEQ ID NO:4), and reverse primers contained the M13 reverse sequence (5′-CAGGAAACAGCTATGACC-3′; SEQ ID NO:5). Locations of primers within the gene were chosen to avoid repetitive sequence. Amplifications were performed with AmpliTaq Gold DNA polymerase (Perkin Elmer, Foster City, Calif.) using a “hot start” to help ensure amplification specificity.

Amplicons were sequenced using an ABI 377 DNA sequencer using BigDye™ (Perkin Elmer) dye-primer sequencing chemistry. Both DNA strands were sequenced. To exclude PCR-induced artifacts, independent amplification followed by DNA sequencing was performed for all samples in which a SNP was only observed once among the samples resequenced. DNA sequence chromatograms were analyzed using the PolyPhred 3.0 (Nickerson et al. (1997) Nucl. Acids Res. 25:2745-2751) and Consed 8.0 (Gordon et al. (1998) Genome Res. 8:195-202) programs developed by the University of Washington (Seattle, Wash.). The University of Wisconsin GCG software package, Version 10, also was used to analyze nucleotide sequence.

SLC6A4 expression constructs and COS-1 cell transfection: A wild type (WT) SLC6A4 sequence (e.g., having a reference sequence set forth in GenBank® accession no. U79746 or NM_{—}001045) is amplified using human cDNA (e.g., human brain cDNA; Clontech) as template and cloned into a eukaryotic expression vector. The WT construct is then used as the template for site-directed mutagenesis, performed using circular PCR to create variant constructs. Sequences of the constructs are confirmed by sequencing both strands of the insert.

Expression constructs are transiently expressed in (e.g., in COS-1) cells using the TransFast reagent (Promega, Madison, Wis.) at a charge ratio of 1:1. Specifically, 10 μg of SLC6A4 expression construct DNA is cotransfected with 2 μg of pSV-β-Galactosidase DNA (Promega) to correct for transfection efficiency. After 48 hours, cells are lysed in 0.1% NP40 lysis buffer, followed by centrifugation at 3000g for 10 minutes. Supernatants are used for Western blot analysis.

SLC6A4 transporter activity: SLC6A4 activity is measured using the method described by Kendall et al (supra). Briefly, [³H]serotonin transport assays are carried between 19 and 24 hours post transfection. Cells are washed with phosphate-buffered saline containing 0.1 mM CaCl₂ and 1 mM MgCl₂ (PBSCM). Transport is measured by incubating the cells with [1,2-³H]serotonin (NEN Life Science Products) in 80 μl of PBSCM for 15 minutes at room temperature, an interval that includes only the initial, linear phase of transport. Each well is washed very quickly three times with ice-cold phosphate-buffered saline. The cells are lysed in 100 μl of 1% SDS, transferred to scintillation vials, and counted in 3 ml of Optifluor (Packard Instrument Co.). The transport activity of each mutant is tested multiple times (e.g., at least two different times), in duplicate wells each time, and the results are averaged.

SLC6A4 reporter gene constructs and luciferase assay: Luciferase reporter gene constructs are created for the most common human SLC6A4 5′-FR haplotypes (e.g., haplotypes having a frequency ≧3% in any of the populations). Specifically, a segment containing about 1000 bp of the SLC6A4 5′-FR is amplified from human genomic DNA samples that contains the desired haplotypes. Forward and reverse primers include restriction sites to enable subcloning of the amplicons into a reporter construct (e.g., pGL-3 Basic (Promega)), upstream of the firefly luciferase gene open reading frame (ORF). The insert is sequenced in both directions to ensure that the correct sequence is present.

Luciferase reporter gene constructs are used to transiently transfect cell lines demonstrated to have SLC6A4 luciferase activity. Cells are transfected with 5 μg purified plasmid DNA with 50 ng pRL-TK (Promega) DNA. Renilla luciferase activity expressed by pRL-TK is used as a control for transfection efficiency. Cells also are transfected with pGL-3 Basic without insert as a control. Transfection is performed using the TransFast reagent (Promega). After 48 hours, cells are lysed and reporter gene activity is measured using a Promega dual-luciferase assay system. Results are reported as the ratio of firefly luciferase light units to Renilla luciferase light units, and all values are expressed as a percentage of the activity of the pGL3 WT 5′-FR construct.

Western blot analysis: Supernatants of COS-1 cell lysates containing expressed WT and variant expression constructs are used for Western blot analysis. After correction for β-galactosidase activity, cell lysates are loaded on 12% SDS PAGE and transferred onto PVDF membrane (BioRad Laboratories, Hercules, Calif.), followed by blotting with a monoclonal anti-His antibody (Sigma, St Louis, Mo.). Results are quantified using the AMBIS Radioanalytic Imaging System, Wuant Probe Version 4.31 (Ambis, Inc., San Diego, Calif.), and the data are expressed as a percentage of the intensity of the WT SLC6A4.

In vitro translation and degradation: Transcription and translation of SLC6A4 allozymes is performed using the TNT coupled RRL System (Promega). Specifically, 1 μg expression construct is added to 25 μL RRL that has been treated to inhibit protein degradation, together with 2 μL T7 buffer, 1 μL T7 polymerase, 1 μL of a mixture of amino acids that lacks methionine, 1 μL RNasin, and 2 μL ³⁵S-methionine (1000 Ci/mM, 10 mCi/mL, 0.4 μM final concentration). With the exception of the RNasin (Promega) and ³⁵S-methionine (Amersham Pharmacia Biotech), all reagents are included in the Promega kit. The reaction volume is increased to 50 μL with nuclease-free water (Promega), and the mixture is incubated at 30° C. for 90 minutes. A 5 μL aliquot is used to perform SDS-PAGE, followed by autoradiography.

For protein degradation experiments, 10 μL of in vitro translated ³⁵S-methionine-labeled protein is added to 50 μL of an adenosine 5′-triphosphate (ATP) generating system and 50 μL of “untreated” RRL. The ATP generating system contains 100 μL each of 1 M Tris-HCl (pH 7.8), 160 mM MgCl₂, 120 mM KCl, 100 mM dithiothreitol, 100 mM ATP, 200 mM creatine phosphate, and 2 mg/mL creatine kinase (all from Sigma), plus 300 μL nuclease-free water (Promega). This mixture is incubated at 37° C., and aliquots are removed at various time points (e.g., at 0, 4, and 8 hours) for SDS-PAGE followed by autoradiography. ³⁵S-methionine radioactively-labeled protein is quantified using the AMBIS System.

Quantitative RT-PCR: mRNA is isolated from cells cotransfected with a control β-Galactosidase reporter and an SLC6A4 WT expression construct or an SLC6A4 variant expression construct using an RNeasy Mini Kit (Qiagen, Valencia, Calif.), according to the manufacturer's instructions. RT-PCR is performed with primers for both SLC6A4 and 5-Galactosidase as an internal control.

Data Analysis: Statistical comparison of the data was performed by ANOVA using the StatView program, version 4.5 (Abacus Concepts, Inc., Berkeley, Calif.). Linkage analysis was performed after all DNA samples were genotyped at each of the polymorphic sites observed, using the EH program developed by Terwilliger and Ott, Handbook of Human Genetic Linkage, The Johns Hopkins University Press, Baltimore, pp. 188-193 (1994). D′ values, a quantitative method for reporting linkage data that is independent of allele frequency (Hartl and Clark Principles of Population Genetics, 3^{rd} edition, Sinauer Associates, Inc., (Sunderland, Mass.), pp. 96-106 (1997); and Hedrick Genetics of Populations, 2^{nd} edition, Jones and Bartlett (Sudbury, Mass.), pp. 396-405 (2000)), were calculated. The genotype data also was used to assign inferred haplotypes using a program based on the E-M algorithm (Long et al. (1995) Am. J. Hum. Genet. 56:799-810; and Excoffier and Slatkin (1995) Mol. Biol. Evol. 12:921-927). Unambiguous haplotype assignment was possible on the basis of genotype for samples that contained no more than one heterozygous polymorphism.

### Example 2

### SLC6A4 Polymorphisms

PCR amplifications were performed for each of the DNA samples studied. PCR amplicons were sequenced on both strands, making it possible to verify the presence of polymorphisms using data from the complimentary strand. A total of 53 polymorphisms were observed (Table 3). Polymorphisms in exons and flanking regions (FR) are numbered relative to the adenine in the SLC6A4 translation initiation codon (ATG, adenine is +1). Polymorphisms in introns are numbered separately, either as positive numbers relative to the guanine in the splice donor site (GT, guanine is +1), or as negative numbers relative to the guanine in the splice acceptor site (AG, guanine is −1).

Variant allele frequencies ranged from 0.8% to 93.0%, with differences between the African-American (AA), Caucasian-American (CA), Han Chinese-American (HCA), and Mexican-American (MA) subjects. Thirty-two SNPs were observed in AA subjects, 23 in CA subjects, 11 in HCA subjects, and 20 in MA subjects. Eighteen of these polymorphisms were specific to AA subjects, 10 to CA subjects, 4 to HCA subjects, and 5 to MA subjects.

Eight SNPs were observed within the coding-region (cSNPs), and five of those cSNPs—located in exons 2, 10, 11, and 13—were nonsynonymous, resulting in the amino acid alterations Gly41Ala, Gly56Ala, Phe465Leu, Val488Met, and Lys605Asn. The Gly41Ala polymorphism had a frequency of 0.8% in Han Chinese-Americans but was not observed in DNA from African-American, Caucasian-American, or Mexican-American subjects. The Gly56Ala polymorphism had a frequency of 3.3% in Caucasian-Americans, but was not observed in African-Americans, Han Chinese-Americans, or Mexican-Americans. The Phe465Leu and Val488Met polymorphism had a frequency of 0.8% in African-Americans, but was not observed in DNA from Caucasian-Americans, Han Chinese-Americans, or Mexican-Americans. The Lys605Asn polymorphism had a frequency of 2.5% in Han Chinese-Americans, but was not observed in African-Americans, Caucasian-Americans, or Mexican-Americans. To exclude artifacts introduced by PCR-dependent misincorporation, independent amplifications were performed and the amplicons were sequenced in all cases in which a polymorphism was observed only once among the DNA samples studied. All polymorphisms were in Hardy-Weinberg equilibrium (P>0.05).

### Example 3

### Linkage Disequilibrium Analysis and Haplotype Analysis

Linkage disequilibrium analysis was performed after all of the DNA samples had been genotyped at each of the polymorphic sites. Pairwise combinations of these polymorphisms were tested for linkage disequilibrium using the EH program developed by Terwilliger and Ott, supra. The output of this program was used to calculate d′ values, a method for reporting linkage data that is independent of sample size. Pairwise combinations with a statistically significant linkage disequilibrium (P value <0.001) are shown in Tables 4-7. D′ values greater than 0 indicate a positive association, while d′ values less than 0 indicate a negative association.

The genotype data also were used for haplotype analysis. Haplotypes typically are defined as combinations of alleles on a single chromosome, although they are sometimes referred to in a more restricted definition as all polymorphisms present on a single allele (Altshuler et al. (2005) Nature 437:1299-1320). Haplotypes can be determined unequivocally if not more than one polymorphism per allele is heterozygous, but haplotypes also can be inferred computationally (Schaid et al. (2002) Am. J. Hum. Genet. 70:425-434). SLC6A4 haplotypes, both observed and inferred, with frequencies >0.08%, are listed in Tables 8-11. As shown in the tables, the identified haplotypes accounted for 85% of all DNA samples from Caucasian-American subjects, 75% of all DNA samples from African-American subjects, 94% of all DNA samples from Han Chinese-American subjects, and 88% of all DNA samples from Mexican-American subjects.

TABLE 3
SLC6A4 Polymorphisms and Frequencies

| | | Position in | Position in | Sequence | Amino acid | | | | |
|---|---|---|---|---|---|---|---|---|---|
| Location | Nucleotide | SEQ ID 6 | SEQ ID 7 | change | change | AA | CA | HCA | MA |
| 5′FR | −3791 | 7607 | | C→T | | 0.008 | 0.000 | 0.000 | 0.000 |
| 5′FR | −3780 | 7618 | | G→C | | 0.008 | 0.000 | 0.000 | 0.000 |
| 5′FR | −3745 | 7653 | | T→A | | 0.042 | 0.000 | 0.000 | 0.000 |
| 5′FR | −3684 | 7714 | | C→T | | 0.033 | 0.000 | 0.000 | 0.025 |
| 5′FR | −3636 | 7762 | | T→C | | 0.110 | 0.000 | 0.000 | 0.000 |
| 5′FR | −3631 | 7767 | | G→A | | 0.025 | 0.050 | 0.000 | 0.042 |
| 5′FR | −2063 to | 9335 to | | 44 bp | | 0.377 | 0.404 | 0.712 | 0.644 |
| | −1714 | 9684 | | deletion | | | | | |
| 5′FR | −1090 | 10308 | | A→T | | 0.017 | 0.000 | 0.000 | 0.000 |
| 5′FR | −1089 | 10309 | | A→T | | 0.373 | 0.067 | 0.060 | 0.050 |
| 5′FR | −859 | 10539 | | A→C | | 0.083 | 0.000 | 0.000 | 0.000 |
| 5′FR | −650 | 10748 | | C→A | | 0.000 | 0.008 | 0.000 | 0.000 |
| 5′FR | −482 | 10916 | | T→C | | 0.083 | 0.075 | 0.150 | 0.058 |
| 5′FR | −469 | 10929 | | C→T | | 0.000 | 0.042 | 0.000 | 0.000 |
| Intron 1a | −47 | 23828 | | G→C | | 0.008 | 0.033 | 0.000 | 0.033 |
| Intron 1a | −45 | 23830 | | C→A | | 0.475 | 0.800 | 0.900 | 0.868 |
| Intron 1a | −25 | 23850 | | G→A | | 0.000 | 0.025 | 0.000 | 0.000 |
| 5′UTR | −185 | 23910 | | A→C | | 0.325 | 0.200 | 0.108 | 0.125 |
| 5′UTR | −149 | 23946 | | C→A | | 0.108 | 0.000 | 0.000 | 0.025 |
| Intron 1b | 28 | 23999 | | G→A | | 0.000 | 0.033 | 0.000 | 0.000 |
| Intron 1b | 112 | 24083 | | C→T | | 0.000 | 0.008 | 0.000 | 0.008 |
| Intron 1b | 162 | 24133 | | G→A | | 0.000 | 0.008 | 0.000 | 0.000 |
| Intron 1b | 242 | 24213 | | T→C | | 0.000 | 0.000 | 0.000 | 0.008 |
| Exon 2 | 122 | 24953 | 122 | G→C | Gly41Ala | 0.000 | 0.000 | 0.008 | 0.000 |
| Exon 2 | 167 | 24998 | 167 | G→C | Gly56Ala | 0.000 | 0.033 | 0.000 | 0.000 |
| Exon 2 | 303 | 25134 | 303 | T→C | | 0.025 | 0.008 | 0.000 | 0.000 |
| Intron 2 | | 25201 to | | VNTR₉ | | 0.000 | 0.018 | 0.000 | 0.000 |
| | | 25403 | | | | | | | |
| | | | | VNTR₁₀ | | 0.319 | 0.436 | 0.070 | 0.276 |
| | | | | VNTR₁₂ | | 0.681 | 0.545 | 0.930 | 0.724 |
| Exon 3 | 411 | 27926 | 411 | C→T | | 0.000 | 0.000 | 0.000 | 0.008 |
| Intron 3 | 11 | 28004 | | G→T | | 0.008 | 0.000 | 0.000 | 0.000 |
| Intron 3 | −105 | 28348 | | A→C | | 0.000 | 0.017 | 0.000 | 0.000 |
| Intron 4 | 10 | 28682 | | C→T | | 0.008 | 0.000 | 0.000 | 0.000 |
| Intron 4 | −100 | 29386 | | G→A | | 0.108 | 0.000 | 0.000 | 0.008 |
| Intron 4 | −86 | 29400 | | C→T | | 0.000 | 0.008 | 0.000 | 0.000 |
| Intron 4 | −85 | 29401 | | G→A | | 0.008 | 0.000 | 0.000 | 0.000 |
| Intron 6 | 56 | 30764 | | G→A | | 0.008 | 0.000 | 0.000 | 0.000 |
| Intron 6 | 67 | 30775 | | A→C | | 0.000 | 0.008 | 0.000 | 0.008 |
| Intron 6 | −115 | 30961 | | C→T | | 0.000 | 0.008 | 0.000 | 0.000 |
| Intron 6 | −44 | 31032 | | G→C | | 0.042 | 0.000 | 0.000 | 0.000 |
| Intron 6 | −37 | 31039 | | C→T | | 0.000 | 0.000 | 0.008 | 0.000 |
| Intron 7 | 83 | 31262 | | C→C | | 0.017 | 0.025 | 0.000 | 0.008 |
| Intron 7 | −24 | 33899 | | G→A | | 0.008 | 0.000 | 0.000 | 0.000 |
| Exon 8 | 1149 | 33995 | 1149 | C→T | | 0.000 | 0.000 | 0.000 | 0.033 |
| Intron 8 | 204 | 34254 | | T→G | | 0.000 | 0.000 | 0.008 | 0.000 |
| Intron 8 | 220 | 34270 | | C→T | | 0.025 | 0.000 | 0.000 | 0.000 |
| Intron 9 | −25 | 36119 | | G→A | | 0.067 | 0.000 | 0.000 | 0.000 |
| Exon 10 | 1393 | 36219 | 1393 | T→C | Phe465Leu | 0.008 | 0.000 | 0.000 | 0.000 |
| Intron 10 | 69 | 36344 | | C→T | | 0.000 | 0.000 | 0.000 | 0.008 |
| Intron 10 | −40 | 37508 | | C→G | | 0.017 | 0.000 | 0.000 | 0.000 |
| Exon 11 | 1462 | 37560 | 1462 | G→A | Val488Met | 0.008 | 0.000 | 0.000 | 0.000 |
| Intron 11 | −131 | 38827 | | C→T | | 0.066 | 0.000 | 0.000 | 0.000 |
| Intron 11 | −118 | 38840 | | C→T | | 0.000 | 0.000 | 0.000 | 0.008 |
| Exon 13 | 1815 | 43615 | 1815 | A→C | Lys605Asn | 0.000 | 0.000 | 0.025 | 0.000 |

TABLE 4
Caucasian American SLC6A4 Linkage Disequilibrium Statistics

| Location 1 | Location 2 | D′ Value | P-Value |
|---|---|---|---|
| 5′FR (−3631) | 5′FR (−1089) | 0.8164 | 0.000 |
| 5′FR (−3631) | Intron 1a (−47) | 0.7296 | 0.000 |
| 5′FR (−1089) | Intron 1a (−47) | 0.7194 | 0.0002 |
| 5′FR (−650) | Intron 7 (83) | 1.000 | 0.000 |
| 5′FR (−469) | Intron 1b (28) | 0.4713 | 0.000 |
| 5′FR (−469) | Exon 2 (167) | 0.4713 | 0.000 |
| 5′FR (−469) | Intron 6 (67) | 1.000 | 0.000 |
| Intron 1a (−45) | 5′UTR (−185) | 0.9474 | 0.000 |
| Intron 1b (28) | Exon 2 (167) | 1.000 | 0.000 |
| Intron 1b (28) | Intron 6 (67) | 1.000 | 0.0002 |
| Exon 2 (167) | Intron 6 (67) | 1.000 | 0.0002 |

TABLE 5
African American SLC6A4 Linkage Disequilibrium Statistics

| Location 1 | Location 2 | D′ Value | P-Value |
|---|---|---|---|
| 5′FR (−3780) | 5′FR (−3631) | 1.000 | 0.000 |
| 5′FR (−3780) | Intron 6 (56) | 1.000 | 0.000 |
| 5′FR (−3684) | 5′UTR (−149) | 1.000 | 0.000 |
| 5′FR (−3684) | Exon 10 (1393) | 1.000 | 0.000 |
| 5′FR (−3636) | Intron 9 (−25) | 0.698 | 0.0001 |
| 5′FR (−3631) | Intron 1a (−47) | 1.000 | 0.000 |
| 5′FR (−3631) | Intron 6 (56) | 1.000 | 0.000 |
| 5′FR (−482) | 5′UTR (−149) | 0.646 | 0.0001 |
| Intron 1a (−45) | 5′UTR (−185) | 1.000 | 0.000 |
| Intron 1a (−45) | VNTR | 0.7695 | 0.0001 |
| Intron 1a (−45) | Intron 4 (−100) | 1.000 | 0.0002 |
| 5′UTR (−185) | VNTR | 0.8547 | 0.0007 |
| 5′UTR (−185) | Intron 4 (−100) | 1.000 | 0.000 |
| 5′UTR (−149) | Exon 10 (1393) | 1.000 | 0.0001 |
| Intron 4 (−85) | Intron 6 (−44) | 1.000 | 0.0008 |
| Intron 4 (−85) | Intron 8 (220) | 1.000 | 0.000 |
| Intron 4 (−85) | Intron 10 (−40) | 1.000 | 0.000 |
| Intron 6 (−44) | Intron 8 (220) | 1.000 | 0.000 |

TABLE 6
Han Chinese American SLC6A4 Linkage Disequilibrium Statistics

| Location 1 | Location 2 | D′ Value | P-Value |
|---|---|---|---|
| 5HTTLPR | VNTR | 0.8045 | 0.0002 |
| Intron 1a (−45) | 5′UTR (−185) | 1.000 | 0.000 |

TABLE 7
Mexican American SLC6A4 Linkage Disequilibrium Statistics

| Location 1 | Location 2 | D′ Value | P-Value |
|---|---|---|---|
| 5′FR (−3684) | Intron 4 (−100) | 1.000 | 0.000 |
| 5′FR (−3631) | 5′FR (−1089) | 1.000 | 0.000 |
| 5′FR (−3631) | Intron 1a (−47) | 1.000 | 0.000 |
| 5′FR (−3631) | Intron 6 (67) | 1.000 | 0.0008 |
| 5′FR (−3631) | Intron 10 (69) | 1.000 | 0.0008 |
| 5′FR (−1089) | Intron 1a (−47) | 1.000 | 0.000 |
| Intron 1a (−47) | Intron 6 (67) | 1.000 | 0.0002 |
| Intron 1a (−47) | Intron 10 (69) | 1.000 | 0.0002 |
| Intron 1a (−45) | 5′UTR (−185) | 1.000 | 0.000 |

TABLE 8
SLC6A4 Haplotypes for Caucasian American Population

| | Observed | | | | | | | | | | | |
|---|---|---|---|---|---|---|---|---|---|---|---|---|
| | or | 5′FR | 5′FR | 5′FR | 5′FR | 5′FR | 5′FR | | 5′FR | 5′FR | 5′FR | 5′FR |
| Frequency | Inferred | (−3791) | (−3780) | (−3745) | (−3684) | (−3636) | (−3631) | HTTLPR | (−1090) | (−1089) | (−859) | (−650) |
| 25% | o | C | G | T | C | T | G | 490 | A | A | A | C |
| 17% | o | C | G | T | C | T | G | 533 | A | A | A | C |
| 17% | o | C | G | T | C | T | G | 533 | A | A | A | C |
| 6% | o | C | G | T | C | T | G | 490 | A | A | A | C |
| 4% | i | C | G | T | C | T | G | 533 | A | A | A | C |
| 4% | i | C | G | T | C | T | G | 490 | A | A | A | C |
| 3% | i | C | G | T | C | T | [symbol] | 533 | A | [symbol] | A | C |
| 2% | i | C | G | T | C | T | G | 533 | A | A | A | C |
| 2% | i | C | G | T | C | T | G | 533 | A | A | A | C |
| 2% | i | C | G | T | C | T | G | 490 | A | A | A | C |
| 2% | i | C | G | T | C | T | G | 533 | A | A | A | C |
| 1% | i | C | G | T | C | T | G | 533 | A | A | A | C) |

| | | | Intron | Intron | Intron | | | Intron | Intron | Intron | Intron | | |
|---|---|---|---|---|---|---|---|---|---|---|---|---|---|
| | 5′FR | 5′FR | 1a | 1a | 1a | 5′UTR | 5′UTR | 1b | 1b | 1b | 1b | Exon 2 | Exon 2 |
| Frequency | (−482) | (−469) | (−47) | (−45) | (−25) | (−185) | (−149) | (28) | (112) | (162) | (242) | (122) | (167) |
| 25% | T | C | G | [symbol] | G | A | C | G | C | G | T | G | G |
| 17% | T | C | G | [symbol] | G | A | C | G | C | G | T | G | G |
| 17% | T | C | G | [symbol] | G | A | C | G | C | G | T | G | G |
| 6% | T | C | G | [symbol] | G | A | C | G | C | G | T | G | G |
| 4% | T | C | G | C | G | [symbol] | C | G | C | G | T | G | G |
| 4% | T | C | G | C | G | [symbol] | C | G | C | G | T | G | G |
| 3% | T | C | [symbol] | C | G | [symbol] | C | G | C | G | T | G | G |
| 2% | [symbol] | C | G | C | G | [symbol] | C | G | C | G | T | G | G |
| 2% | T | C | G | [symbol] | G | A | C | G | C | G | T | G | G |
| 2% | T | C | G | [symbol] | G | A | C | [symbol] | C | G | T | G | [symbol] |
| 2% | [symbol] | C | G | [symbol] | G | A | C | G | C | G | T | G | G |
| 1% | [symbol] | C | G | [symbol] | G | A | C | G | C | G | T | G | G) |
| | Exon 2 | | Exon 3 | Intron 3 | Intron 3 | Intron 4 | Intron 4 | Intron 4 | Intron 4 | Intron 6 | Intron 6 | Intron 6 | Intron 6 |
| Frequency | (303) | VNTR | (411) | (11) | (−105) | (10) | (−100) | (−86) | (−85) | (56) | (67) | (−115) | (−44) |
| 25% | T | 12 | C | G | A | C | G | C | G | G | A | C | G |
| 17% | T | 12 | C | G | A | C | G | C | G | G | A | C | G |
| 17% | T | 10 | C | G | A | C | G | C | G | G | A | C | G |
| 6% | T | 10 | C | G | A | C | G | C | G | G | A | C | G |
| 4% | T | 12 | C | G | A | C | G | C | G | G | A | C | G |
| 4% | T | 10 | C | G | A | C | G | C | G | G | A | C | G |
| 3% | T | 10 | C | G | A | C | G | C | G | G | A | C | G |
| 2% | T | 12 | C | G | A | C | G | C | G | G | A | C | G |
| 2% | T | 10 | C | G | [symbol] | C | G | C | G | G | A | C | G |
| 2% | T | 10 | C | G | A | C | G | C | G | G | A | C | G |
| 2% | T | 10 | C | G | A | C | G | C | G | G | A | C | G |
| 1% | T | 9 | C | G | A | C | G | C | G | G | A | C | G) |

| | | | | | | | | Exon | Intron | Intron | Exon | Intron | Intron | Exon |
|---|---|---|---|---|---|---|---|---|---|---|---|---|---|---|
| | Intron 6 | Intron 7 | Intron 7 | Exon 8 | Intron 8 | Intron 8 | Intron 9 | 10 | 10 | 10 | 11 | 11 | 11 | 13 |
| Frequency | (−37) | (83) | (−24) | (1149) | (204) | (220) | (−25) | (1393) | (69) | (−40) | (1462) | (−131) | (−118) | (1815) |
| 25% | C | C | G | C | T | C | G | T | C | C | G | C | C | A |
| 17% | C | C | G | C | T | C | G | T | C | C | G | C | C | A |
| 17% | C | C | G | C | T | C | G | T | C | C | G | C | C | A |
| 6% | C | C | G | C | T | C | G | T | C | C | G | C | C | A |
| 4% | C | C | G | C | T | C | G | T | C | C | G | C | C | A |
| 4% | C | C | G | C | T | C | G | T | C | C | G | C | C | A |
| 3% | C | C | G | C | T | C | G | T | C | C | G | C | C | A |
| 2% | C | C | G | C | T | C | G | T | C | C | G | C | C | A |
| 2% | C | C | G | C | T | C | G | T | C | C | G | C | C | A |
| 2% | C | C | G | C | T | C | G | T | C | C | G | C | C | A |
| 2% | C | C | G | C | T | C | G | T | C | C | G | C | C | A |
| 1% | C | C | G | C | T | C | G | T | C | C | G | C | C | A |

TABLE 9
SLC6A4 Haplotypes for African American Population

| | Observed | | | | | | | | | | | |
|---|---|---|---|---|---|---|---|---|---|---|---|---|
| | or | 5′FR | 5′FR | 5′FR | 5′FR | 5′FR | 5′FR | | 5′FR | 5′FR | 5′FR | 5′FR |
| Frequency | Inferred | (−3791) | (−3780) | (−3745) | (−3684) | (−3636) | (−3631) | HTTLPR | (−1090) | (−1089) | (−859) | (−650) |
| 14% | o | C | G | T | C | T | G | 533 | A | A | A | C |
| 8% | o | C | G | T | C | T | G | 490 | A | [symbol] | A | C |
| 7% | i | C | G | T | C | T | G | 533 | A | A | A | C |
| 6% | i | C | G | T | C | T | G | 490 | A | A | A | C |
| 5% | i | C | G | T | C | [symbol] | G | 490 | A | [symbol] | A | C |
| 4% | o | C | G | T | C | T | G | 533 | A | A | A | C |
| 4% | i | C | G | T | C | T | G | 533 | A | A | A | C |
| 4% | i | C | G | T | C | [symbol] | G | 490 | A | [symbol] | A | C |
| 3% | i | C | G | T | C | T | G | 533 | A | A | [symbol] | C |
| 3% | i | C | G | T | C | T | G | 533 | A | [symbol] | A | C |
| 3% | i | C | G | T | C | T | G | 533 | A | A | A | C |
| 3% | i | C | G | T | C | T | G | 533 | A | [symbol] | A | C |
| 2% | i | C | G | T | C | T | G | 490 | A | [symbol] | A | C |
| 2% | i | C | G | [symbol] | C | T | G | 490 | A | A | A | C |
| 2% | i | C | G | T | C | T | G | 533 | A | A | [symbol] | C |
| 1% | i | C | G | T | C | T | G | 490 | A | A | A | C |
| 1% | i | C | G | T | C | T | G | 533 | A | A | A | C |
| 1% | i | C | G | T | C | T | G | 533 | A | A | A | C |
| 1% | i | C | G | T | C | T | G | 533 | A | [symbol] | A | C |
| 1% | i | C | G | T | C | T | G | 533 | A | A | A | C |

| | | | Intron | Intron | Intron | | | Intron | Intron | Intron | Intron | | |
|---|---|---|---|---|---|---|---|---|---|---|---|---|---|
| | 5′FR | 5′FR | 1a | 1a | 1a | 5′UTR | 5′UTR | 1b | 1b | 1b | 1b | Exon 2 | Exon 2 |
| Frequency | (−482) | (−469) | (−47) | (−45) | (−25) | (−185) | (−149) | (28) | (112) | (162) | (242) | (122) | (167) |
| 14% | T | C | G | [symbol] | G | A | C | G | C | G | T | G | G |
| 8% | T | C | G | C | G | [symbol] | C | G | C | G | T | G | G |
| 7% | T | C | G | C | G | [symbol] | C | G | C | G | T | G | G |
| 6% | T | C | G | [symbol] | G | A | C | G | C | G | T | G | G |
| 5% | T | C | G | [symbol] | G | A | C | G | C | G | T | G | G |
| 4% | T | C | G | [symbol] | G | A | C | G | C | G | T | G | G |
| 4% | [symbol] | C | G | C | G | A | [symbol] | G | C | G | T | G | G |
| 4% | T | C | G | C | G | [symbol] | C | G | C | G | T | G | G |
| 3% | T | C | G | [symbol] | G | A | C | G | C | G | T | G | G |
| 3% | T | C | G | [symbol] | G | A | C | G | C | G | T | G | G |
| 3% | T | C | G | [symbol] | G | A | C | G | C | G | T | G | G |
| 3% | T | C | G | C | G | [symbol] | C | G | C | G | T | G | G |
| 2% | T | C | G | C | G | [symbol] | C | G | C | G | T | G | G |
| 2% | T | C | G | C | G | A | C | G | C | G | T | G | G |
| 2% | T | C | G | C | G | [symbol] | C | G | C | G | T | G | G |
| 1% | T | C | G | C | G | [symbol] | C | G | C | G | T | G | G |
| 1% | T | C | G | C | G | [symbol] | C | G | C | G | T | G | G |
| 1% | T | C | G | C | G | [symbol] | C | G | C | G | T | G | G |
| 1% | T | C | G | C | G | A | C | G | C | G | T | G | G |
| 1% | T | C | G | C | G | A | C | G | C | G | T | G | G |
| | Exon 2 | | Exon 3 | Intron 3 | Intron 3 | Intron 4 | Intron 4 | Intron 4 | Intron 4 | Intron 6 | Intron 6 | Intron 6 | Intron 6 |
| Frequency | (303) | VNTR | (411) | (11) | (−105) | (10) | (−100) | (−86) | (−85) | (56) | (67) | (−115) | (−44) |
| 14% | T | 10 | C | G | A | C | G | C | G | G | A | C | G |
| 8% | T | 12 | C | G | A | C | A | C | G | G | A | C | G |
| 7% | T | 12 | C | G | A | C | G | C | G | G | A | C | G |
| 6% | T | 12 | C | G | A | C | G | C | G | G | A | C | G |
| 5% | T | 10 | C | G | A | C | G | C | G | G | A | C | G |
| 4% | T | 12 | C | G | A | C | G | C | G | G | A | C | G |
| 4% | T | 12 | C | G | A | C | G | C | G | G | A | C | G |
| 4% | T | 12 | C | G | A | C | G | C | G | G | A | C | G |
| 3% | T | 12 | C | G | A | C | G | C | G | G | A | C | G |
| 3% | T | 10 | C | G | A | C | G | C | G | G | A | C | G |
| 3% | T | 10 | C | G | A | C | G | C | G | G | A | C | G |
| 3% | T | 12 | C | G | A | C | G | C | G | G | A | C | G |
| 2% | T | 12 | C | G | A | C | G | C | G | G | A | C | G |
| 2% | T | 12 | C | G | A | C | G | C | G | G | A | C | C |
| 2% | T | 12 | C | G | A | C | G | C | G | G | A | C | G |
| 1% | T | 12 | C | G | A | C | G | C | G | G | A | C | G |
| 1% | T | 10 | C | G | A | C | G | C | G | G | A | C | G |
| 1% | T | 12 | C | G | A | C | [symbol] | C | G | G | A | C | G |
| 1% | [symbol] | 12 | C | G | A | C | G | C | G | G | A | C | G |
| 1% | T | 10 | C | G | A | C | G | C | G | G | A | C | G |

| | | | | | | | | Exon | Intron | Intron | Exon | Intron | Intron | Exon |
|---|---|---|---|---|---|---|---|---|---|---|---|---|---|---|
| | Intron 6 | Intron 7 | Intron 7 | Exon 8 | Intron 8 | Intron 8 | Intron 9 | 10 | 10 | 10 | 11 | 11 | 11 | 13 |
| Frequency | (−37) | (83) | (−24) | (1149) | (204) | (220) | (−25) | (1393) | (69) | (−40) | (1462) | (−131) | (−118) | (1815) |
| 14% | C | C | G | C | T | C | G | T | C | C | G | C | C | A |
| 8% | C | C | G | C | T | C | G | T | C | C | G | C | C | A |
| 7% | C | C | G | C | T | C | G | T | C | C | G | C | C | A |
| 6% | C | C | G | C | T | C | G | T | C | C | G | C | C | A |
| 5% | C | C | G | C | T | C | [symbol] | T | C | C | G | C | C | A |
| 4% | C | C | G | C | T | C | G | T | C | C | G | C | C | A |
| 4% | C | C | G | C | T | C | G | T | C | C | G | C | C | A |
| 4% | C | C | G | C | T | C | G | T | C | C | G | C | C | A |
| 3% | C | C | G | C | T | C | G | T | C | C | G | C | C | A |
| 3% | C | C | G | C | T | C | G | T | C | C | G | C | C | A |
| 3% | C | C | G | C | T | C | G | T | C | C | G | [symbol] | C | A |
| 3% | C | C | G | C | T | C | G | T | C | C | G | C | C | A |
| 2% | C | C | G | C | T | C | G | T | C | C | G | C | C | A |
| 2% | C | C | G | C | T | C | G | T | C | C | G | C | C | A |
| 2% | C | C | G | C | T | C | G | T | C | C | G | C | C | A |
| 1% | C | C | G | C | T | C | G | T | C | C | G | C | C | A |
| 1% | C | C | G | C | T | C | G | T | C | C | G | C | C | A |
| 1% | C | C | G | C | T | C | G | T | C | C | G | C | C | A |
| 1% | C | C | G | C | T | C | G | T | C | C | G | C | C | A |
| 1% | C | C | G | C | T | C | G | T | C | C | G | C | C | A |

TABLE 10
SLC6A4 Haplotypes for Han Chinese American Population

| | Observed | | | | | | | | | | | |
|---|---|---|---|---|---|---|---|---|---|---|---|---|
| | or | 5′FR | 5′FR | 5′FR | 5′FR | 5′FR | 5′FR | | 5′FR | 5′FR | 5′FR | 5′FR |
| Frequency | Inferred | (−3791) | (−3780) | (−3745) | (−3684) | (−3636) | (−3631) | HTTLPR | (−1090) | (−1089) | (−859) | (−650) |
| 60% | o | C | G | T | C | T | G | 490 | A | A | A | C |
| 7% | o | C | G | T | C | T | G | 533 | A | A | A | C |
| 6% | i | C | G | T | C | T | G | 533 | A | A | A | C |
| 5% | o | C | G | T | C | T | G | 490 | A | A | A | C |
| 3% | i | C | G | T | C | T | G | 533 | A | [symbol] | A | C |
| 3% | i | C | G | T | C | T | G | 490 | A | A | A | C |
| 3% | i | C | G | T | C | T | G | 533 | A | A | A | C |
| 2% | i | C | G | T | C | T | G | 533 | A | [symbol] | A | C |
| 2% | o | C | G | T | C | T | G | 490 | A | A | A | C |
| 1% | i | C | G | T | C | T | G | 533 | A | A | A | C |
| 1% | i | C | G | T | C | T | G | 533 | A | A | A | C |
| 1% | i | C | G | T | C | T | G | 533 | A | A | A | C |

| | | | Intron | Intron | Intron | | | Intron | Intron | Intron | Intron | | |
|---|---|---|---|---|---|---|---|---|---|---|---|---|---|
| | 5′FR | 5′FR | 1a | 1a | 1a | 5′UTR | 5′UTR | 1b | 1b | 1b | 1b | Exon 2 | Exon 2 |
| Frequency | (−482) | (−469) | (−47) | (−45) | (−25) | (−185) | (−149) | (28) | (112) | (162) | (242) | (122) | (167) |
| 60% | T | C | G | [symbol] | G | A | C | G | C | G | T | G | G |
| 7% | T | C | G | [symbol] | G | A | C | G | C | G | T | G | G |
| 6% | [symbol] | C | G | [symbol] | G | A | C | G | C | G | T | G | G |
| 5% | [symbol] | C | G | [symbol] | G | A | C | G | C | G | T | G | G |
| 3% | T | C | G | C | G | [symbol] | C | G | C | G | T | G | G |
| 3% | T | C | G | C | G | [symbol] | C | G | C | G | T | G | G |
| 3% | T | C | G | [symbol] | G | A | C | G | C | G | T | G | G |
| 2% | T | C | G | [symbol] | G | A | C | G | C | G | T | G | G |
| 2% | T | C | G | [symbol] | G | A | C | G | C | G | T | G | G |
| 1% | T | C | G | C | G | [symbol] | C | G | C | G | T | G | G |
| 1% | [symbol] | C | G | [symbol] | G | A | C | G | C | G | T | G | G |
| 1% | [symbol] | C | G | C | G | [symbol] | C | G | C | G | T | G | G |
| | Exon 2 | | Exon 3 | Intron 3 | Intron 3 | Intron 4 | Intron 4 | Intron 4 | Intron 4 | Intron 6 | Intron 6 | Intron 6 | Intron 6 |
| Frequency | (303) | VNTR | (411) | (11) | (−105) | (10) | (−100) | (−86) | (−85) | (56) | (67) | (−115) | (−44) |
| 60% | T | 12 | C | G | A | C | G | C | G | G | A | C | G |
| 7% | T | 12 | C | G | A | C | G | C | G | G | A | C | G |
| 6% | T | 12 | C | G | A | C | G | C | G | G | A | C | G |
| 5% | T | 12 | C | G | A | C | G | C | G | G | A | C | G |
| 3% | T | 12 | C | G | A | C | G | C | G | G | A | C | G |
| 3% | T | 12 | C | G | A | C | G | C | G | G | A | C | G |
| 3% | T | 10 | C | G | A | C | G | C | G | G | A | C | G |
| 2% | T | 12 | C | G | A | C | G | C | G | G | A | C | G |
| 2% | T | 12 | C | G | A | C | G | C | G | G | A | C | G |
| 1% | T | 12 | C | G | A | C | G | C | G | G | A | C | G |
| 1% | T | 10 | C | G | A | C | G | C | G | G | A | C | G |
| 1% | T | 12 | C | G | A | C | G | C | G | G | A | C | G |

| | | | | | | | | Exon | Intron | Intron | Exon | Intron | Intron | Exon |
|---|---|---|---|---|---|---|---|---|---|---|---|---|---|---|
| | Intron 6 | Intron 7 | Intron 7 | Exon 8 | Intron 8 | Intron 8 | Intron 9 | 10 | 10 | 10 | 11 | 11 | 11 | 13 |
| Frequency | (−37) | (83) | (−24) | (1149) | (204) | (220) | (−25) | (1393) | (69) | (−40) | (1462) | (−131) | (−118) | (1815) |
| 60% | C | C | G | C | T | C | G | T | C | C | G | C | C | A |
| 7% | C | C | G | C | T | C | G | T | C | C | G | C | C | A |
| 6% | C | C | G | C | T | C | G | T | C | C | G | C | C | A |
| 5% | C | C | G | C | T | C | G | T | C | C | G | C | C | A |
| 3% | C | C | G | C | T | C | G | T | C | C | G | C | C | A |
| 3% | C | C | G | C | T | C | G | T | C | C | G | C | C | A |
| 3% | C | C | G | C | T | C | G | T | C | C | G | C | C | A |
| 2% | C | C | G | C | T | C | G | T | C | C | G | C | C | A |
| 2% | C | C | G | C | T | C | G | T | C | C | G | C | C | [symbol] |
| 1% | C | C | G | C | T | C | G | T | C | C | G | C | C | A |
| 1% | C | C | G | C | T | C | G | T | C | C | G | C | C | A |
| 1% | C | C | G | C | T | C | G | T | C | C | G | C | C | A |

TABLE 11
SLC6A4 Haplotypes for Mexican American Population

| | Observed | | | | | | | | | | | |
|---|---|---|---|---|---|---|---|---|---|---|---|---|
| | or | 5′FR | 5′FR | 5′FR | 5′FR | 5′FR | 5′FR | | 5′FR | 5′FR | 5′FR | 5′FR |
| Frequency | Inferred | (−3791) | (−3780) | (−3745) | (−3684) | (−3636) | (−3631) | HTTLPR | (−1090) | (−1089) | (−859) | (−650) |
| 51% | o | C | G | T | C | T | G | 490 | A | A | A | C |
| 13% | o | C | G | T | C | T | G | 533 | A | A | A | C |
| 6% | o | C | G | T | C | T | G | 533 | A | A | A | C |
| 6% | o | C | G | T | C | T | G | 490 | A | A | A | C |
| 2% | o | C | G | T | C | T | G | 533 | A | A | A | C |
| 2% | i | C | G | T | C | T | G | 490 | A | A | A | C |
| 2% | i | C | G | T | C | T | G | 490 | A | A | A | C |
| 2% | i | C | G | T | C | T | G | 533 | A | A | A | C |
| 2% | i | C | G | T | C | T | [symbol] | 533 | A | [symbol] | A | C |
| 1% | i | C | G | T | C | T | G | 533 | A | A | A | C |
| 1% | i | C | G | T | C | T | G | 533 | A | A | A | C |

| | | | Intron | Intron | Intron | | | Intron | Intron | Intron | Intron | | |
|---|---|---|---|---|---|---|---|---|---|---|---|---|---|
| | 5′FR | 5′FR | 1a | 1a | 1a | 5′UTR | 5′UTR | 1b | 1b | 1b | 1b | Exon 2 | Exon 2 |
| Frequency | (−482) | (−469) | (−47) | (−45) | (−25) | (−185) | (−149) | (28) | (112) | (162) | (242) | (122) | (167) |
| 51% | T | C | G | [symbol] | G | A | C | G | C | G | T | G | G |
| 13% | T | C | G | [symbol] | G | A | C | G | C | G | T | G | G |
| 6% | T | C | G | [symbol] | G | A | C | G | C | G | T | G | G |
| 6% | T | C | G | [symbol] | G | A | C | G | C | G | T | G | G |
| 2% | T | C | G | C | G | [symbol] | C | G | C | G | T | G | G |
| 2% | T | C | G | C | G | [symbol] | C | G | C | G | T | G | G |
| 2% | T | C | G | [symbol] | G | A | C | G | C | G | T | G | G |
| 2% | [symbol] | C | G | [symbol] | G | A | C | G | C | G | T | G | G |
| 2% | T | C | C | C | G | [symbol] | C | G | C | G | T | G | G |
| 1% | T | C | G | [symbol] | G | A | C | G | C | G | T | G | G |
| 1% | [symbol] | C | G | C | G | [symbol] | C | G | C | G | T | G | G |
| | Exon 2 | | Exon 3 | Intron 3 | Intron 3 | Intron 4 | Intron 4 | Intron 4 | Intron 4 | Intron 6 | Intron 6 | Intron 6 | Intron 6 |
| Frequency | (303) | VNTR | (411) | (11) | (−105) | (10) | (−100) | (−86) | (−85) | (56) | (67) | (−115) | (−44) |
| 51% | T | 12 | C | G | A | C | G | C | G | G | A | C | G |
| 13% | T | 10 | C | G | A | C | G | C | G | G | A | C | G |
| 6% | T | 12 | C | G | A | C | G | C | G | G | A | C | G |
| 6% | T | 10 | C | G | A | C | G | C | G | G | A | C | G |
| 2% | T | 12 | C | G | A | C | G | C | G | G | A | C | G |
| 2% | T | 12 | C | G | A | C | G | C | G | G | A | C | G |
| 2% | T | 10 | C | G | A | C | G | C | G | G | A | C | G |
| 2% | T | 10 | C | G | A | C | G | C | G | G | A | C | G |
| 2% | T | 12 | C | G | A | C | G | C | G | G | A | C | G |
| 1% | T | 10 | C | G | A | C | G | C | G | G | A | C | G |
| 1% | T | 12 | C | G | A | C | G | C | G | G | A | C | G |

| | | | | | | | | Exon | Intron | Intron | Exon | Intron | Intron | Exon |
|---|---|---|---|---|---|---|---|---|---|---|---|---|---|---|
| | Intron 6 | Intron 7 | Intron 7 | Exon 8 | Intron 8 | Intron 8 | Intron 9 | 10 | 10 | 10 | 11 | 11 | 11 | 13 |
| Frequency | (−37) | (83) | (−24) | (1149) | (204) | (220) | (−25) | (1393) | (69) | (−40) | (1462) | (−131) | (−118) | (1815) |
| 51% | C | C | G | C | T | C | G | T | C | C | G | C | C | A |
| 13% | C | C | G | C | T | C | G | T | C | C | G | C | C | A |
| 6% | C | C | G | C | T | C | G | T | C | C | G | C | C | A |
| 6% | C | C | G | C | T | C | G | T | C | C | G | C | C | A |
| 2% | C | C | G | C | T | C | G | T | C | C | G | C | C | A |
| 2% | C | C | G | C | T | C | G | T | C | C | G | C | C | A |
| 2% | C | C | G | [symbol] | T | C | G | T | C | C | G | C | C | A |
| 2% | C | C | G | C | T | C | G | T | C | C | G | C | C | A |
| 2% | C | C | G | C | T | C | G | T | C | C | G | C | C | A |
| 1% | C | C | G | [symbol] | T | C | G | T | C | C | G | C | C | A |
| 1% | C | C | G | C | T | C | G | T | C | C | G | C | C | A |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for determining an SLC6A4 genotype, comprising
a) providing a nucleic acid sample from a subject, and
b) determining in said nucleic acid sample whether or not one or more genetic markers are in linkage disequilibrium with an SLC6A4 allele, wherein said SLC6A4 allele is selected from the group consisting of the alleles set forth in Table 1.

2. The method of claim 1, wherein said SLC6A4 allele is selected from the group consisting of an rs25531a allele, an rs25531g allele, an rs25532c allele, and an rs25532t allele.

3. The method of claim 1, wherein said SLC6A4 allele is a 5HTTLPR short allele.

4. A method for determining an SLC6A4 genotype, comprising
a) providing a nucleic acid sample from a subject, and
b) determining in said nucleic acid sample whether or not one or more genetic markers are in linkage disequilibrium with an SLC6A4 allele, wherein said SLC6A4 allele is selected from the group consisting of the alleles set forth in Table 3.

5. The method of claim 4, wherein said SLC6A4 allele is selected from the group consisting of a G122C allele, a G167C allele, a T1393C allele, a G1462A allele, or an A1815C allele.

6. An isolated nucleic acid molecule consisting essentially of at least fifteen contiguous nucleotides of SEQ ID NO:6, wherein said sequence includes at least one nucleotide selected from the group consisting of: nucleotide 7607 of SEQ ID NO:6, with the proviso that the nucleotide at position 7607 is thymine; nucleotide 7618 of SEQ ID NO:6, with the proviso that the nucleotide at position 7618 is cytosine; nucleotide 7653 of SEQ ID NO:6, with the proviso that the nucleotide at position 7653 is adenine; nucleotide 7714 of SEQ ID NO:6, with the proviso that the nucleotide at position 7714 is thymine; nucleotide 7762 of SEQ ID NO:6, with the proviso that the nucleotide at position 7762 is cytosine; nucleotide 10308 of SEQ ID NO:6, with the proviso that the nucleotide at position 10308 is thymine; nucleotide 10748 of SEQ ID NO:6, with the proviso that the nucleotide at position 10748 is adenine; nucleotide 10929 of SEQ ID NO:6, with the proviso that the nucleotide at position 10929 is thymine; nucleotide 24133 of SEQ ID NO:6, with the proviso that the nucleotide at position 24133 is adenine; nucleotide 24213 of SEQ ID NO:6, with the proviso that the nucleotide at position 24213 is cytosine; nucleotide 24953 of SEQ ID NO:6, with the proviso that the nucleotide at position 24953 is cytosine; nucleotide 25134 of SEQ ID NO:6, with the proviso that the nucleotide at position 25134 is cytosine; nucleotide 27926 of SEQ ID NO:6, with the proviso that the nucleotide at position 27926 is thymine; nucleotide 28004 of SEQ ID NO:6, with the proviso that the nucleotide at position 28004 is thymine; nucleotide 28682 of SEQ ID NO:6, with the proviso that the nucleotide at position 28682 is thymine; nucleotide 29386 of SEQ ID NO:6, with the proviso that the nucleotide at position 29386 is adenine; nucleotide 29400 of SEQ ID NO:6, with the proviso that the nucleotide at position 29400 is thymine; nucleotide 29401 of SEQ ID NO:6, with the proviso that the nucleotide at position 29401 is adenine; nucleotide 30775 of SEQ ID NO:6, with the proviso that the nucleotide at position 30775 is cytosine; nucleotide 30961 of SEQ ID NO:6, with the proviso that the nucleotide at position 30961 is thymine; nucleotide 31039 of SEQ ID NO:6, with the proviso that the nucleotide at position 31039 is thymine; nucleotide 33899 of SEQ ID NO:6, with the proviso that the nucleotide at position 33899 is adenine; nucleotide 33995 of SEQ ID NO:6, with the proviso that the nucleotide at position 33995 is thymine; nucleotide 34254 of SEQ ID NO:6, with the proviso that the nucleotide at position 34254 is guanine; nucleotide 34270 of SEQ ID NO:6, with the proviso that the nucleotide at position 34270 is thymine; nucleotide 36119 of SEQ ID NO:6, with the proviso that the nucleotide at position 36119 is adenine; nucleotide 36344 of SEQ ID NO:6, with the proviso that the nucleotide at position 36344 is thymine; nucleotide 37560 of SEQ ID NO:6, with the proviso that the nucleotide at position 37560 is adenine; nucleotide 38827 of SEQ ID NO:6, with the proviso that the nucleotide at position 38827 is thymine; and nucleotide 38840 of SEQ ID NO:6, with the proviso that the nucleotide at position 38840 is thymine;
or the complement of said sequence.

7. The isolated nucleic acid of molecule of claim 6, wherein said isolated nucleic acid molecule is 15 to 100 nucleotides in length.

8. The isolated nucleic acid molecule of claim 6, wherein said isolated nucleic acid molecule is 20 to 50 nucleotides in length.

9. A vector comprising the isolated nucleic acid molecule of claim 6.

10. The vector of claim 9, wherein said nucleic acid molecule is 20 to 50 nucleotides in length.

11. An isolated nucleic acid encoding a SLC6A4 polypeptide comprising an amino acid sequence variant relative to the amino acid sequence set forth in SEQ ID NO:3, wherein said amino acid sequence variant is at residue 122 or residue 1462.

12. The isolated nucleic acid of claim 11, wherein said amino acid sequence variant is an alanine at residue 122 or a methionine at residue 1462.

13. An isolated SLC6A4 polypeptide, wherein said polypeptide comprises an amino acid sequence variant relative to the amino acid sequence set forth in SEQ ID NO:3, and wherein said amino acid sequence variant is at residue 122 or 1462.

14. The isolated polypeptide of claim 13, wherein said amino acid sequence variant is an alanine at residue 122 or a methionine at residue 1462.

15. An article of manufacture comprising a substrate, wherein said substrate comprises a population of isolated nucleic acid molecules of claim 6.

16. The article of manufacture of claim 15, wherein said substrate comprises a plurality of discrete regions, wherein each said region comprises a different population of isolated nucleic acid molecules, and wherein each said population of molecules comprises a different SLC6A4 nucleotide sequence variant.
